(19) United States
(12) Patent Application Publication
(10) Pub. No.: US 2006/0195017 A1
(43) Pub. Date: Aug. 31, 2006

(54) Expandable device for providing access to the spine

(76) Inventors: Alan E. Shluzas, West Roxbury, MA (US); Gene P. DiPoto, Upton, MA (US); Christopher P. DeGeorge, Franklin, MA (US); Stephen J. Anderson, Holliston, MA (US); Jeffrey Williams, Plainville, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/285,226

(22) Filed: Nov. 22, 2005

Related U.S. Application Data
(60) Provisional application No. 60/630,180, filed on Nov. 22, 2004.

(51) Int. Cl. A61B 1/32

(52) U.S. Cl. 600/210

(57) ABSTRACT

In one embodiment, a retractor is provided for retracting tissue at a surgical location within a patient for minimally invasive access to a region of the spine. The retractor comprises an elongate body having a proximal portion and a distal portion. The retractor comprises a first elongate member and a second elongate member. The first and second elongate members define an outer surface of the retractor. The retractor is actuatable between a low profile configuration and an expanded configuration.

### PRIORITY INFORMATION

This application is based on and claims the priority of U.S. Provisional Patent Application No. 60/630,180, filed on Nov. 22, 2004, which is hereby expressly incorporated by reference herein in its entirety.

### BACKGROUND OF THE INVENTION

1. Field of the Invention

This application is directed to an access assembly for a surgical system that can be actuated from a low-profile configuration for insertion to an enlarged configuration after being inserted.

2. Description of the Related Art

Spinal surgery presents significant difficulties to the physician attempting to reduce chronic back pain or correct spinal deformities without introducing additional trauma due to the surgical procedure itself. In order to access the vertebrae to perform spinal procedures, the physician is typically required to make large incisions and cut or strip muscle tissue surrounding the spine. In addition, care must be taken not to injure nerve tissue in the area. Consequently, traditional surgical procedures of this type carry high risks of scarring, pain, significant blood loss, and extended recovery times.

Apparatuses for performing minimally invasive techniques have been proposed to reduce the trauma of posterior spinal surgery by reducing the size of the incision and the degree of muscle stripping in order to access the vertebrae. Such apparatuses had taken the form of narrow tubes or cannulae that are inserted through relatively small incisions. Such cannulae are too small for many procedures that are performed using open surgery techniques.

### SUMMARY OF THE INVENTION

There is a need in the art for systems and methods for treating the spine that provide minimally invasive access to the spine such that a variety of procedures, and preferably the entire procedure or at least a substantial portion thereof, can be performed via an access device.

In one technique, a method is provided for treating a spine of a patient. An elongate body is inserted into the patient through an incision. The elongate body comprises a distal portion that extends along a longitudinal axis, a proximal portion, and an outer surface. The elongate body is expanded to retract tissue beneath the incision. An access device is inserted over the elongate body. The access device comprises a distal portion, a proximal portion, an outer surface, and an inner surface. The access device is advanced until the distal end resides at or near a region of the spine.

In another embodiment, a system for providing access to a surgical location adjacent to a spine of a patient is provided. The system includes a dilator and an access device. The dilator has a proximal portion, a first elongate member, a second elongate member, and a movable member at least partially located between the first and second elongate members. The proximal portion is coupled with and is configured to move the movable member to move the dilator to an expanded configuration. The access device has a passage that extends therethrough. The passage is configured such that the access device can be advanced over the dilator when the dilator is in the expanded configuration.

In another embodiment, a system for providing access to a surgical location adjacent to a spine of a patient is provided. The system includes a dilator assembly and an access device. The dilator assembly comprises a first elongate body and a second elongate body. The first elongate body comprises a first puzzle feature on an outside surface thereof configured to join the first elongate body to another elongate body. The second elongate body comprises a first side portion and a second side portion. The first and second side portions are configured to be inserted over the first elongate body and to substantially surround the first elongate body. At least one of the first and second side portions comprises a second puzzle feature configured to join the second elongate body to another elongate body. The access device has a passage extending therethrough. The access device is capable of being configured such that the passage has a shape corresponding to the shape of the second elongate body. The outer perimeter of the second elongate body is configured such that the access device can be advanced over the dilator assembly in use.

In another embodiment, a retractor is provided for retracting tissue at a surgical location within a patient for minimally invasive access to a region of the spine. The retractor comprises an elongate body having a proximal portion and a distal portion. The retractor comprises a first elongate member and a second elongate member. The first and second elongate members define an outer surface of the retractor. The retractor is actuatable between a low profile configuration and an expanded configuration.

In one embodiment, a retractor is provided for retracting tissue at a surgical location within a patient for minimally invasive access to a region of the spine. The retractor comprises an elongate body having a proximal portion, a first elongate member, and a second elongate member. The first and second elongate members are configured to retract tissues to expose at least a portion of at least one vertebra. The elongate body has an expanded configuration wherein a recess is defined at least in part by the first elongate member and the second elongate member.

In one embodiment, a retractor is provided for retracting tissue at a surgical location within a patient. The retractor comprises an elongate body having a proximal portion, a distal portion, a first side portion, and a second side portion. The first side portion has a first longitudinal edge. The second side portion has a second longitudinal edge. The first and second side portions are movable relative to each other such that the first and second longitudinal edges can be positioned in close proximity to each other or spaced apart by a selected distance. The elongate body has an outer surface and an inner surface. The inner surface at least partially defines a passage. The elongate body is capable of having a low profile configuration and an expanded configuration when positioned within the patient. The cross-sectional area defined by the outer surface in the expanded configuration is greater than the cross-sectional area defined by the outer surface in the low-profile configuration.

In one application, a method for retracting tissue at a surgical location within a patient comprises providing a retractor for insertion into the patient. The retractor has a proximal portion and a distal portion. The retractor has a first longitudinal edge on a first side portion and a second longitudinal edge on a second side portion. The retractor has an outer surface. The retractor is inserted into the patient to the surgical location with the first and second longitudinal edges of the proximal portion positioned in close proximity to each other. The retractor is configured such that the first and second longitudinal edges are spaced apart by a selected distance. The retractor is configured such that the cross-sectional area defined by the outer surface is expanded.

In another embodiment, a system provides access to a surgical location adjacent the spine. The system comprises a first retractor for retracting tissue at the surgical location. The first retractor comprises an elongate body having a proximal portion, a distal portion, an outer surface, an inner surface, a first elongate member, and a second elongate member. The first and second elongate members are configured to retract tissues to expose at least a portion of at least one vertebra. The outer surface is defined at least in part by the first elongate member and the second elongate member. The elongate body has low profile configuration and an expanded configuration. The cross-sectional area defined by the outer surface in the expanded configuration is greater than the cross-sectional area defined by the outer surface in the low-profile configuration. The system comprises a second retractor for providing minimally invasive access to the spine. The second retractor comprises an elongate body having an outer surface and an inner surface. The inner surface defines a passage extending through the elongate body. The second retractor is capable of having a configuration wherein the inner surface of the second retractor is positionable over the outer surface of the first retractor when the first retractor is in the expanded configuration within the patient. The elongate body of the second retractor is capable of having a configuration when positioned within the patient wherein the cross-sectional area of the passage at a first location is greater than the cross-sectional area of the passage at a second location, wherein the first location is distal to the second location. The passage is capable of having a configuration through which multiple surgical instruments can be inserted simultaneously to the surgical location when the first retractor is withdrawn from the passage.

In one application, a method for providing treatment at or near a region of the spine of a patient is provided. An incision is formed in the skin of a patient. A first expandable elongate body is inserted into the patient through the incision. The first expandable elongate body has a distal portion, a proximal portion, an outer surface, and an inner surface. The first expandable elongate body is advanced until a distal end thereof resides at or near a region of the spine. A proximal end of the proximal portion remains outside the patient. The first expandable elongate body is expanded to retract tissue. A second expandable elongate body is inserted into the patient over the expanded first expandable elongate body. The second expandable elongate body has a distal portion, a proximal portion, an outer surface, and an inner surface. The second expandable elongate body is advanced until a distal end thereof resides at or near a region of the spine. The first expandable elongate body is withdrawn from the patient. The second expandable elongate body is expanded to retract tissue.

In another application, a method for accessing a surgical location within a patient comprises providing a first retractor for insertion into the patient. The first retractor has a first elongate member and a second elongate member. The first retractor is positioned in a low-profile configuration for insertion into the patient. In the low-profile configuration, the first elongate member is adjacent the second elongate member. The first retractor is positioned in an enlarged profile configuration. In the enlarged profile configuration, the first elongate member is spaced from the second elongate member. A second retractor is provided for insertion into the patient. The second retractor has a proximal portion and a distal portion. The distal portion is coupled with the proximal portion and has an outer surface and an inner surface partially defining a passage. The second retractor is positioned in a low-profile configuration for insertion into the patient. The second retractor is inserted into the patient to the surgical location over the first retractor. The second retractor is positioned in an enlarged profile configuration. In the enlarged profile configuration, the second retractor is configured such that the cross-sectional area of the passage at a first location is greater than the cross-sectional area of said passage at a second location, wherein the first location is distal to the second location.

In another embodiment, a system provides access to a surgical location adjacent the spine. The system comprises a first dilator for retracting tissue at the surgical location. The first dilator comprises an elongate body having a proximal portion, a distal portion, and an outer surface. In some embodiments, the first dilator has an inner surface defining a bore. The elongate body is configured to retract tissues when inserted within a patient. In some embodiments the first dilator is expandable. In other embodiments, the first dilator is not expandable. The system comprises a second dilator for retracting tissue at the surgical location. The second dilator comprises an elongate body having a proximal portion, a distal portion, and an outer surface. In some embodiments, the second dilator has an inner surface defining a bore. The elongate body is configured to retract tissues when inserted within a patient. In some embodiments the second dilator is expandable. In other embodiments, the second dilator is not expandable. In some embodiments the first and second dilators can have an oblong or round cross-sectional shape. The system comprises an access device having an outer surface and an inner surface. The inner surface defines a passage extending through the elongate body. The access device is capable of having a configuration wherein the inner surface of the access device is positionable over the outer surface of the one or more of the retractors when the one or more retractors are positioned within the patient. The elongate body of the access device is capable of having a configuration when positioned within the patient wherein the cross-sectional area of the passage at a first location is greater than the cross-sectional area of the passage at a second location, wherein the first location is distal to the second location. The passage is capable of having a configuration through which multiple surgical instruments can be inserted simultaneously to the surgical location when the first retractor is withdrawn from the passage.

In one application, a method for providing treatment at or near a region of the spine of a patient is provided. A first dilator is inserted into the patient through an incision to retract tissue. The first dilator is advanced until a distal end thereof resides at or near a region of the spine. A second dilator is inserted into the patient through the incision to retract tissue. The second dilator is advanced until a distal end thereof resides at or near a region of the spine. An expandable access device is positioned over one or more of the first and second dilators. The expandable access device is expanded to retract tissue. In one variation of the application, the first dilator and the second dilator can have an oblong or round cross-sectional shape. In another variation of the application, the first dilator and the second dilator are positioned side by side within the patient. In another variation of the application, the first dilator is positioned in and removed from the patient before the second dilator is positioned in the patient. In another variation of the application, the first dilator and the second dilator have inner surfaces defining bores of approximately the same size. In another variation of the application, one or more of the first dilator and the second dilator do not have a bore.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a perspective view of one embodiment of a surgical system and one application for treating the spine of a patient.

FIG. 2 is a perspective view of one embodiment of an access device in a reduced profile configuration.

FIG. 3 is a perspective view of the access device of FIG. 2 in a first enlarged configuration.

FIG. 4 is a perspective view of the access device of FIG. 2 in a second enlarged configuration.

FIG. 5 is a view of one embodiment of a skirt portion of an access device.

FIG. 6 is a view of another embodiment of a skirt portion of an access device.

FIG. 7 is a perspective view of another embodiment of an access device.

FIG. 8 is a side view of the access device of FIG. 7.

FIG. 9 is a front view of the access device of FIG. 7.

FIG. 10 is a bottom view of the access device of FIG. 7.

FIG. 11 is a perspective view of the access device of FIG. 7 in a first configuration.

FIG. 12 is an exploded perspective view of the access device of FIG. 7 in a second configuration.

FIG. 13 is a sectional view illustrating one stage of one application for treating the spine of a patient.

FIG. 14 is a side view of one embodiment of an expander apparatus in a reduced profile configuration.

FIG. 15 is a side view of the expander apparatus of FIG. 14 in an expanded configuration.

FIG. 16 is a sectional view of the expander apparatus of FIGS. 14-15 inserted into the access device of FIG. 2, which has been inserted into a patient.

FIG. 17 is a sectional view of the expander apparatus of FIGS. 14-15 inserted into the access device of FIG. 2 and expanded to the expanded configuration to retract tissue.

FIG. 18 is an exploded perspective view of one embodiment of an endoscope mount platform.

FIG. 19 is a top view of the endoscope mount platform of FIG. 18 coupled with one embodiment of an indexing arm and one embodiment of an endoscope.

FIG. 20 is a side view of the endoscope mount platform of FIG. 18 illustrated with one embodiment of an indexing arm and one embodiment of an endoscope.

FIG. 21 is a perspective view of one embodiment of an indexing collar of the endoscope mount platform FIG. 18.

FIG. 22 is a perspective view of one embodiment of an endoscope.

FIG. 23A is a top perspective view of one embodiment of an access system.

FIG. 23B is a side perspective view of the access system of FIG. 23A.

FIG. 23C is a top view of the access system of FIG. 23A.

FIG. 24A is a perspective view of one embodiment of a lighting element.

FIG. 24B is a perspective view of another embodiment of a lighting element.

FIG. 24C is a perspective view of another embodiment of a lighting element.

FIG. 25 is a partial sectional view of one stage of one application of a method for treating the spine of a patient.

FIG. 26 is a perspective view of one embodiment of a fastener.

FIG. 27 is an exploded perspective view of the fastener of FIG. 26.

FIG. 27A is an enlarged side view of one embodiment of a biasing member illustrated in FIG. 27 taken from the perspective of the arrow 27A.

FIG. 28 is a perspective view of one embodiment of a surgical instrument.

FIG. 29 is an enlarged sectional view of the fastener of FIGS. 26-27 coupled with the surgical instrument of FIG. 28, illustrating one stage of one application for treating the spine of a patient.

FIG. 30 is side view of one embodiment of another surgical instrument.

FIG. 31 is a partial sectional view of one stage of one application for treating the spine of a patient.

FIG. 32 is a side view of one embodiment of another surgical instrument.

FIG. 33 is a perspective view similar to FIG. 31 illustrating the apparatuses of FIGS. 26 and 32, in one stage of one application for treating the spine of a patient.

FIG. 34 is an enlarged sectional view of the apparatus of FIGS. 26 and 32, illustrating one stage of one application for treating the spine of a patient.

FIG. 35 is an enlarged sectional similar to FIG. 34, illustrating one stage of one application for treating the spine of a patient.

FIG. 36 is an enlarged view in partial section illustrating one stage of one application for treating the spine of a patient.

FIG. 37 is a partial view of illustrating one stage of one application for treating the spine of a patient.

FIG. 38 is a perspective view of a spinal implant or fusion device constructed according to another embodiment showing a first side surface of the spinal implant.

FIG. 39 is a perspective view of the spinal implant of FIG. 38 showing a second side surface of the spinal implant.

FIG. 40 is a plan view of the spinal implant of FIG. 38 showing an upper surface of the spinal implant.

FIG. 41 is a side view of the spinal implant of FIG. 38 showing the first side surface.

FIG. 42 is a cross-sectional view of the spinal implant taken along the line 42-42 in FIG. 41.

FIG. 43 is a perspective view of another embodiment of a spinal implant constructed according to another embodiment showing a first side surface of the spinal implant.

FIG. 44 is a perspective view of the spinal implant of FIG. 43 showing a second side surface of the spinal implant.

FIG. 45 is a plan view of the spinal implant of FIG. 43 showing an upper surface of the spinal implant.

FIG. 46 is a side view of the spinal implant of FIG. 43 showing the first side surface.

FIG. 47 is a cross-sectional view of the spinal implant taken along the line 47-47 in FIG. 46.

FIG. 48 is a view showing a pair of the spinal implants of FIG. 38 in first relative positions between adjacent vertebrae.

FIG. 49 is a view showing a pair of the spinal implants of FIG. 38 in second relative positions between adjacent vertebrae.

FIG. 50 is a view showing the spinal implant of FIG. 43 between adjacent vertebrae.

FIG. 51 is a view showing a spinal implant being inserted between the adjacent vertebrae according to one application.

FIG. 52 is a side view of an apparatus according to another embodiment.

FIG. 53 is a front view of the apparatus of FIG. 52.

FIG. 54 is a top view of the apparatus of FIG. 52.

FIG. 55 is a back view of the apparatus of FIG. 52.

FIG. 56 is a bottom view of the apparatus of FIG. 52.

FIG. 57 is a sectional view of the apparatus of FIG. 52, used in conjunction with additional structure in a patient.

FIG. 58 is a longitudinal sectional view of the apparatus of FIG. 57 taken from line 58-58 of FIG. 57.

FIG. 59 is a transverse sectional view of the apparatus of FIG. 58 taken from line 59-59 of FIG. 58.

FIG. 60 is a sectional view, similar to FIG. 57, illustrating an alternative position of the apparatus of FIG. 52.

FIG. 61 is a sectional view, similar to FIG. 57, illustrating another alternative position of the apparatus of FIG. 52.

FIG. 62 is a transverse sectional view of the apparatus of FIG. 61, taken along lines 62-62 of FIG. 61.

FIG. 63 is a side view, similar to FIG. 52, of another apparatus.

FIG. 64 is a front view, similar to FIG. 55, of the embodiment or FIG. 63.

FIG. 65 is a sectional view, similar to FIG. 57, of the apparatus of FIG. 63, used in conjunction with additional structure in a patient.

FIG. 66 is a transverse sectional view of the apparatus of FIG. 63, taken along lines 66-66 of FIG. 65.

FIG. 67 is a perspective view of an embodiment of a dilator adapted to dilate or retract tissue to prepare tissue to receive a larger access device or retractor, the dilator shown in a low profile configuration.

FIG. 68 is a plan view of the dilator of FIG. 67 in a reduced profile configuration.

FIG. 69 is a front view of the dilator of FIG. 67.

FIG. 70 is a side view of the dilator of FIG. 67.

FIG. 71 is a longitudinal sectional view of the apparatus of FIG. 67 taken from line 71-71 of FIG. 70.

FIG. 72 is a perspective view of the dilator of FIG. 67 in an expanded configuration.

FIG. 73 is a plan view of the dilator of FIG. 67 in an expanded configuration.

FIG. 74 is a front view of the dilator of FIG. 67 in an expanded configuration.

FIG. 75 is a side view of the dilator of FIG. 67 in an expanded configuration.

FIG. 76 is a longitudinal sectional view of the dilator of FIG. 67 in an expanded configuration taken from line 76-76 of FIG. 75.

FIG. 77 is a schematic view of another embodiment of a dilator, the dilator shown in an expanded configuration.

FIG. 78 is a schematic view of another embodiment of a dilator, capable of a first and a second degree of expansion.

FIG. 79 is perspective view of another embodiment of a dilator that includes a mechanism for expanding the size of the dilator from a low-profile configuration to an expanded configuration.

FIG. 80 is a top view of the dilator of FIG. 79.

FIG. 81 is a plan view of the dilator of FIG. 79.

FIG. 82 is a cross-section view of the dilator of FIG. 79, taken along section plane 82-82 shown in FIG. 81.

FIG. 83 is a cross-section view of the dilator of FIG. 79, taken along section plane 83-83 shown in FIG. 81.

FIG. 84 is an exploded view of the dilator of FIG. 79, showing the arrangement of one embodiment of an actuation system that is configured to expand the transverse size of the dilator.

FIG. 85 is a plan view of another embodiment of a dilator that can be expanded to dilate tissue, the dilator being shown in an expanded position.

FIG. 86 is a partial cross-section view of the dilator of FIG. 85, the dilator being shown in an un-expanded position.

FIG. 86A is a partial cross-section view of the dilator of FIG. 85, the dilator being shown in an expanded position.

FIG. 87 is a cross-section view of the dilator of FIG. 85, taken along section plane 87-87, the dilator being shown in an expanded position.

FIG. 88 is an exploded view of the dilator of FIG. 85, showing the arrangement of one embodiment of an actuation system that is configured to expand the transverse size of the dilator.

FIG. 89 is a schematic view of one embodiment of a dilator system adapted to retract or dilate tissue to prepare tissue to receive a larger access device or retractor.

FIG. 90 is a cross sectional view of the dilator system of FIG. 89 taken from line 90-90 of FIG. 89.

FIG. 91 is a perspective view of a dilator assembly that can be at least partially assembled in a patient.

FIG. 92 is an exploded view of the dilator assembly of FIG. 971.

FIG. 93 is a top view of the dilator assembly of FIG. 91.

Throughout the figures, the same reference numerals and characters, unless otherwise stated, are used to denote like features, elements, components or portions of the illustrated embodiments. Moreover, while the subject matter of this application will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF THE PREFERRED EMBODIMENTS

As should be understood in view of the following detailed description, this application is primarily directed to apparatuses and methods providing access to and for treating the spine of a patient. The apparatuses described below provide access to surgical locations at or near the spine and provide a variety of tools useful treating the spine. As discussed further below, such access is further facilitated by providing convenient dilator apparatuses, systems, and dilation techniques as described further below in Section III. Prior to further detailed discussion of the dilator apparatuses, systems, and techniques, a variety of access devices and retractors usable with such dilator apparatuses, systems, and techniques will be discussed, as well as various procedures performable through the access devices.

### I. Systems for Performing Procedures at a Surgical Location

Various embodiments of apparatuses and procedures described herein will be discussed in terms of minimally invasive procedures and apparatuses, e.g., of endoscopic apparatuses and procedures. However, various embodiments may find use in conventional, open, and mini-open procedures. As used herein, the term “proximal,” as is traditional, refers to the end portion of an apparatus that is closest to the operator, while the term “distal” refers to the end portion that is farthest from the operator.

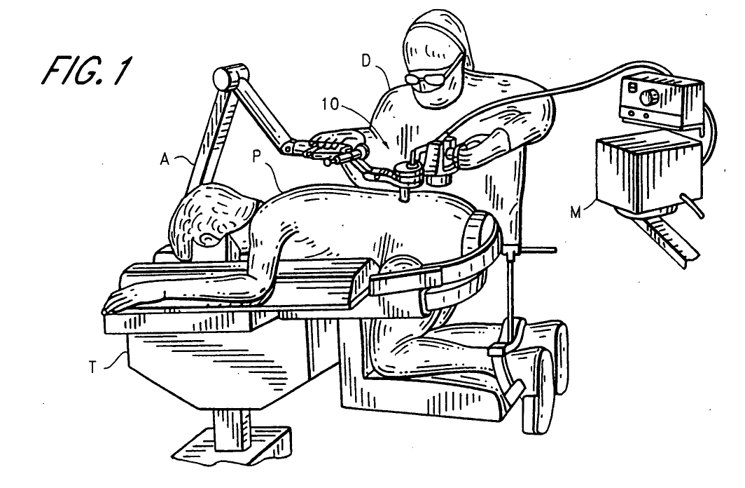

FIG. 1 shows one embodiment of a surgical system 10 that can be used to perform a variety of methods or procedures. In one embodiment, as discussed more fully below, the patient P is placed in the prone position on operating table T, taking care that the abdomen is not compressed and physiological lordosis is preserved. The physician D is able to access the surgical site and perform the surgical procedure with the components of the system 10, which will be described in greater detail herein. The system 10 may be supported, in part, by a mechanical support arm A, such as the type generally disclosed in U.S. Pat. No. 4,863,133, which is hereby incorporated by reference herein in its entirety. One mechanical arm of this type is manufactured by Leonard Medical, Inc., 1464 Holcomb Road, Huntington Valley, Pa., 19006. The mechanical support arm A is sometimes referred to as a “flex arm.” As discussed in greater detail below, the mechanical support arm A is coupled with at least one of an access device and a viewing element.

The term “access device” is used in its ordinary sense to mean a device that can provide access and is a broad term and it includes structures having an elongated dimension and defining a passage, e.g., a cannula or a conduit. The access device is configured to be inserted through the skin of the patient to provide access during a surgical procedure to a surgical location within a patient, e.g., a spinal location. The term “surgical location” is used in its ordinary sense (i.e. a location where a surgical procedure is performed) and is a broad term and it includes locations subject to or affected by a surgery. The term “spinal location” is used in its ordinary sense (i.e. a location at or near a spine) and is a broad term and it includes locations adjacent to or associated with a spine that may be sites for surgical spinal procedures. The access device also can retract tissue to provide greater access to the surgical location. The term “retractor” is used in its ordinary sense to mean a device that can displace tissue and is a broad term and it includes structures having an elongated dimension and defining a passage, e.g., a cannula or a conduit, to retract tissue.

Visualization of the surgical site may be achieved in any suitable manner, e.g., by direct visualization, or by use of a viewing element, such as an endoscope, a camera, loupes, a microscope, or any other suitable viewing element, or a combination of the foregoing. The term “viewing element” is used in its ordinary sense to mean a device useful for viewing and is a broad term and it also includes elements that enhance viewing, such as, for example, a light source or lighting element. In one embodiment, the viewing element provides a video signal representing images, such as images of the surgical site, to a monitol M. The viewing element may be an endoscope and camera that captures images to be displayed on the monitor M whereby the physician D is able to view the surgical site as the procedure is being performed. The endoscope and camera will be described in greater detail herein.

The systems are described herein in connection with minimally invasive postero-lateral spinal surgery. One such procedure is a two level postero-lateral fixation and fusion of the spine involving the L4, L5, and S1 vertebrae. In the drawings, the vertebrae will generally be denoted by reference letter V. The usefulness of the apparatuses and procedures is neither restricted to the postero-lateral approach nor to the L4, L5, and S1 vertebrae. The apparatuses and procedures may be used in other anatomical approaches and with other vertebra(e) within the cervical, thoracic, and lumbar regions of the spine. The procedures may be directed toward surgery involving one or more vertebral levels. Some embodiments are useful for anterior and/or lateral procedures. A retroperitoneal approach can also be used with some embodiments. In one retroperitoneal approach, an initial transverse incision is made just left of the midline, just above the pubis, about 3 centimeters in length. The incision can be carried down through the subcutaneous tissues to the anterior rectus sheath, which is incised transversely and the rectus is retracted medially. At this level, the posterior sheath, where present, can be incised. With blunt finger dissection, the retroperitoneal space can be entered. The space can be enlarged with blunt dissection or with a retroperitoneal balloon dissector. The peritoneal sack can be retracted, e.g., by one of the access devices described herein.

It is believed that embodiments of the invention are also particularly useful where any body structures must be accessed beneath the skin and muscle tissue of the patient. and/or where it is desirable to provide sufficient space and visibility in order to manipulate surgical instruments and treat the underlying body structures. For example, certain features or instrumentation described herein are particularly useful for minimally invasive procedures, e.g., arthroscopic procedures. As discussed more fully below, one embodiment of an apparatus described herein provides an access device that is expandable, e.g., including an expandable distal portion. In addition to providing greater access to a surgical site than would be provided with a device having a constant cross-section from proximal to distal, the expandable distal portion prevents or substantially prevents the access device, or instruments extended therethrough to the surgical site, from dislodging or popping out of the operative site.

A. Systems and Devices for Establishing Access

In one embodiment, the system 10 includes an access device that provides an internal passage for surgical instruments to be inserted through the skin and muscle tissue of the patient P to the surgical site. The access device preferably has a wall portion defining a reduced profile, or low-profile, configuration for initial percutaneous insertion into the patient. This wall portion may have any suitable arrangement. In one embodiment, discussed in more detail below, the wall portion has a generally tubular configuration that may be passed over a dilator that has been inserted into the patient to a traumatically enlarge an opening sufficiently large to receive the access device therein.

The wall portion of the access device preferably can be subsequently expanded to an enlarged configuration, by moving against the surrounding muscle tissue to at least partially define an enlarged surgical space in which the surgical procedures will be performed. The access device may be thought of as a retractor, and may be referred to herein as such. Both the distal and proximal portion may be expanded, as discussed further below. However, the distal portion preferably expands to a greater extent than the proximal portion, because the surgical procedures are to be performed at the surgical site, which is adjacent the distal portion when the access device is inserted into the patient.

While in the reduced profile configuration, the access device preferably defines a first unexpanded configuration. Thereafter, the access device can enlarge the surgical space defined thereby by engaging the tissue surrounding the access device and displacing the tissue outwardly as the access device expands. The access device preferably is sufficiently rigid to displace such tissue during the expansion thereof. The access device may be resiliently biased to expand from the reduced profile configuration to the enlarged configuration. In addition, the access device may also be manually expanded by an expander device with or without one or more surgical instruments inserted therein, as will be described below. The surgical site preferably is at least partially defined by the expanded access device itself. During expansion, the access device can move from a first overlapping configuration to a second overlapping configuration in some embodiments.

In some embodiments, the proximal and distal portions are separate components that may be coupled together in a suitable fashion. For example, the distal end portion of the access device may be configured for relative movement with respect to the proximal end portion in order to allow the physician to position the distal end portion at a desired location. This relative movement also provides the advantage that the proximal portion of the access device nearest the physician D may remain substantially stable during such distal movement. In one embodiment, the distal portion is a separate component that is pivotally or movably coupled to the proximal portion. In another embodiment, the distal portion is flexible or resilient in order to permit such relative movement.

1. Access Devices

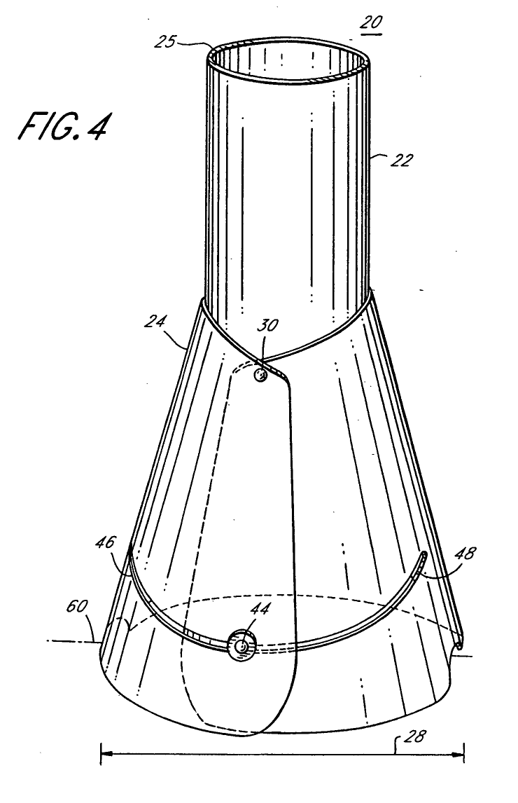

One embodiment of an access device is illustrated in FIGS. 2-6 and designated by reference number 20. In one embodiment, the access device 20 includes a proximal wall portion 22 that has a tubular configuration, and a distal wall portion that has an expandable skirt portion 24. The skirt portion 24 preferably is enlargeable from a reduced profile configuration having an initial dimension 26 (illustrated in FIG. 2) and corresponding cross-sectional area, to an enlarged configuration having a second dimension 28 (illustrated in FIG. 4) and corresponding cross-sectional area. In one embodiment, the skirt portion 24 is coupled to the proximal wall portion 22 with a rivet 30, pin, or similar connecting device to permit movement of the skirt portion 24 relative to the proximal wall portion 22.

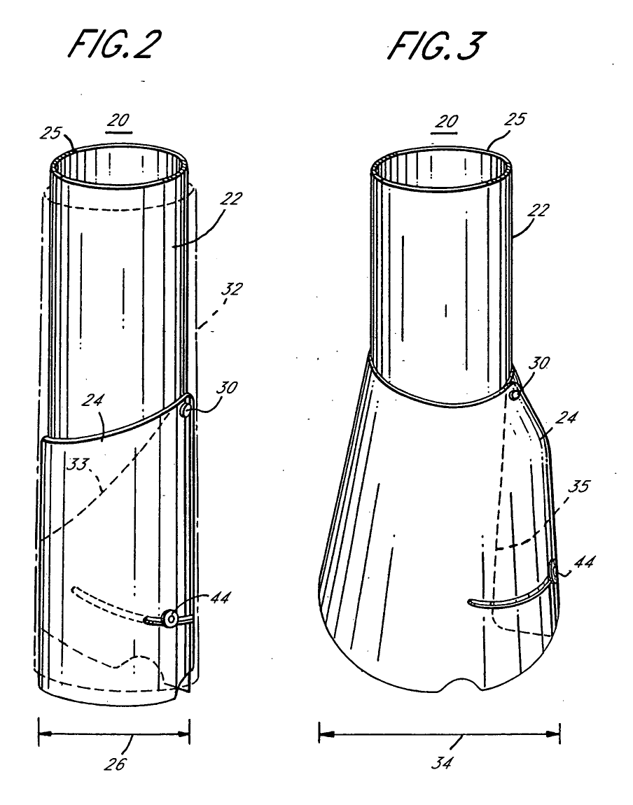

In the illustrated embodiment, the skirt portion 24 is manufactured from a resilient material, such as stainless steel. The skirt portion 24 preferably is manufactured so that it normally assumes an expanded configuration as illustrated in FIG. 4. With reference to FIG. 3, the skirt portion 24 may assume an intermediate dimension 34 and corresponding cross-sectional area, which is greater than the initial dimension 26 of the reduced profile configuration of FIG. 2, and smaller than the dimension 28 of the enlarged configuration of FIG. 4. The skirt portion 24 may assume the intermediate configuration of FIG. 3 when deployed in the patient in response to the force of the tissue acting on the skirt portion 24. The intermediate dimension 34 can depend upon several factors, such as the rigidity of the skirt portion 24, the surrounding tissue, and whether such surrounding tissue has relaxed or tightened during the course of the procedure. An outer sleeve 32 (illustrated in dashed line in FIG. 2) may be provided. Preferably, the outer sleeve surrounds the access device 20 and maintains the skirt portion 24 in the reduced profile configuration prior to insertion into the patient. The outer sleeve 32 may be made of plastic. Where provided, the outer sleeve 32 preferably is configured to be easily deployed. For example, a release device may be provided that releases or removes the outer sleeve 32 upon being operated by the user. In one embodiment, a braided polyester suture is embedded within the sleeve 32, aligned substantially along the longitudinal axis thereof. In use, when the suture is withdrawn, the outer sleeve 32 is torn, allowing the access device 20 to resiliently expand from the reduced profile configuration of FIG. 2 to the expanded configurations of FIGS. 3-4. While in the reduced profile configuration of FIG. 2, the skirt portion 24 defines a first overlapping configuration 33, as illustrated by the dashed line. As the skirt portion 24 resiliently expands, the skirt portion 24 assumes the expanded configuration, as illustrated in FIGS. 3-4.

The skirt portion 24 preferably is sufficiently rigid that it is capable of displacing the tissue surrounding the skirt portion 24 as it expands. Depending Upon the resistance exerted by surrounding tissue, the skirt portion 24 preferably is sufficiently rigid to provide some resistance against the tissue to remain in the configurations of FIGS. 3-4. Moreover, the expanded configuration of the skirt portion 24 is at least partially supported by the body tissue of the patient. The rigidity of the skirt portion 24 and the greater expansion at the distal portion preferably creates a stable configuration that is at least temporarily stationary in the patient. This arrangement preferably frees the physician from the need to actively support the access device 20, e.g., prior to adding an endoscope mount platform 300 and a support arm 400 (see FIGS. 21-22).

One embodiment of the skirt portion 24 of the access device 20 is illustrated in an initial flattened configuration in FIG. 5. The skirt portion 24 may be manufactured from a sheet of stainless steel having a thickness of about 0.007 inches. In various embodiments, the dimension 28 of the skirt portion 24 is about equal to or greater than 50 mm, is about equal to or greater than 60 mm, is about equal to or greater than 70 mm, is about equal to or greater than 80 mm, or is any other suitable size, when the skirt portion 24 is in the enlarged configuration. In one embodiment, the dimension 28 is about 63 mm, when the skirt portion 24 is in the enlarged configuration. The unrestricted shape of the skirt portion 24 is a circular shape in one embodiment and is an oblong shape in another embodiment. In another embodiment, the skirt portion 24 has an oval shape, wherein the dimension 28 defines a longer dimension of the skirt portion 24 and would be about 85 mm. In another embodiment, the skirt portion 24 has an oval shape and the dimension 28 defines a longer dimension of the skirt portion 24 of about 63 mm. An increased thickness, e.g., about 0.010 inches, may be used in connection with skirt portions having a larger diameter, such as about 65 mm. Other materials, such as nitinol or plastics having similar properties, may also be useful.

As discussed above, the skirt portion 24 preferably is coupled to the proximal wall portion 22 with a pivotal connection, such as rivet 30. A pair of rivet holes 36 can be provided in the skirt portion 24 to receive the rivet 30. The skirt portion 24 also has two free ends 38 and 40 in one embodiment that are secured by a slidable connection, such as a second rivet 44 (not shown in FIG. 5, illustrated in FIGS. 2-4). A pair of complementary slots 46 and 48 preferably are defined in the skirt portion 24 adjacent the free ends 38 and 40. The rivet 44 is permitted to move freely within the slots 46 and 48. This slot and rivet configuration allows the skirt portion 24 to move between the reduced profile configuration of FIG. 2 and the enlarged or expanded configurations of FIGS. 3-4. The use of a pair of slots 46 and 48 reduces the risk of the “button-holing” of the rivet 44, e.g., a situation in which the opening of the slot becomes distorted and enlarged such that the rivet may slide out of the slot, and cause failure of the device. The likelihood of such occurrence is reduced in skirt portion 24 because each of the slots 46 and 48 in the double slot configuration has a relatively shorter length than a single slot configuration. Being shorter, the slots 46, 48 are less likely to be distorted to the extent that a rivet may slide out of position. In addition, the configuration of rivet 44 and slots 46 and 48 permits a smoother operation of enlarging and reducing the skirt portion 24, and allows the skirt portion 24 to expand to span three or more vertebrae, e.g., L4, L5, and S1. This arrangement enables multi-level procedures, such as multilevel fixation procedures alone or in combination with a variety of other procedures, as discussed below. Other embodiments include a single slot rather than the slots 46, 48, or more than two slots.

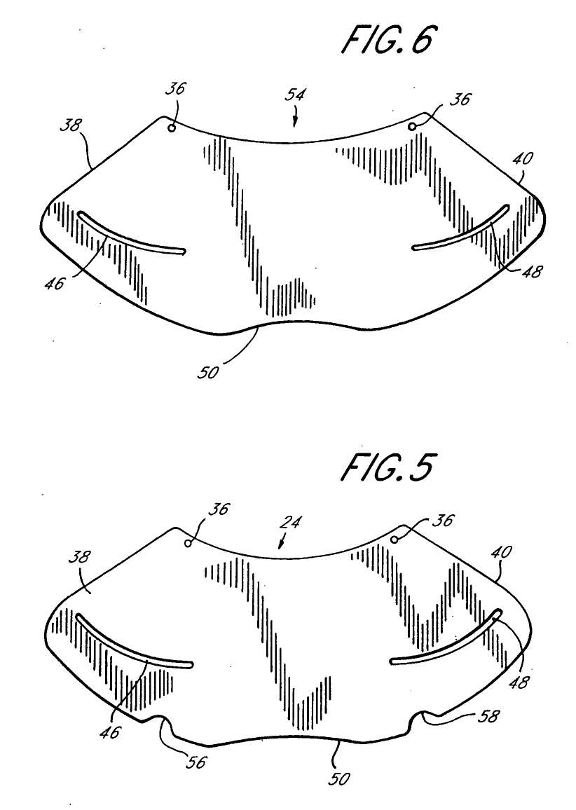

An additional feature of the skirt portion 24 is the provision of a shallow concave profile 50 defined along the distal edge of the skirt portion 24, which allows for improved placement of the skirt portion 24 with respect to the body structures and the surgical instruments defined herein. In one embodiment, a pair of small scalloped or notched portions 56 and 58, are provided, as illustrated in FIG. 5. When the skirt portion 24 is assembled, the notched portions 56 and 58 are generally across from each other. When the skirt portion 24 is applied to a patient, the notched portions 56, 58 are oriented in the ceph-caudal direction (indicated by a dashed line 60 in FIG. 4). In this arrangement, instruments and implants, such as an elongated member 650 used in a fixation procedure (described in detail below), may extend beyond the area enclosed by the skirt portion 24 without moving or raising the skirt portion 24, e.g., by allowing the elongated member 650 (or other implant or instrument) to pass under the skirt portion 24. The notched portions 56, 58 also enable the elongated member 650 (or other implant or instrument) to extend beyond the portion of the surgical space defined within the outline of the distal end of the skirt portion 24. The notched portions 56, 58 are optional, as illustrated in connection with another embodiment of an access device 54, illustrated in FIG. 6, and may be eliminated if, for example, the physician deems the notches to be unnecessary for the procedures to be performed. For example, in some fixation procedures such extended access is not needed, as discussed more fully below. As illustrated in FIG. 4, the skirt portion 24 may be expanded to a substantially conical configuration having a substantially circular or elliptical profile.

Furthermore, it is contemplated that the skirt portion 24 of the access device 20 can include a stop that retains the skirt portion in an expanded configuration, as shown in U.S. patent application Ser. No. 10/361,887, filed Feb. 10, 2003, now U.S. Application Patent Publication No. US2003/153927 A1, which is hereby incorporated by reference in its entirety herein.

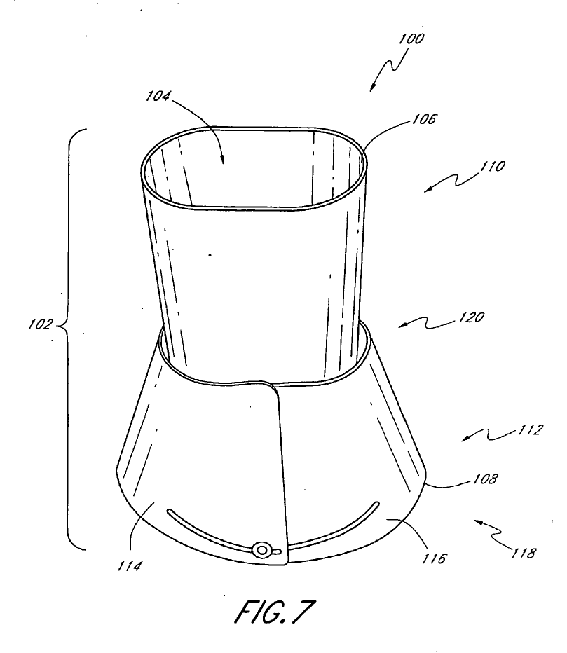

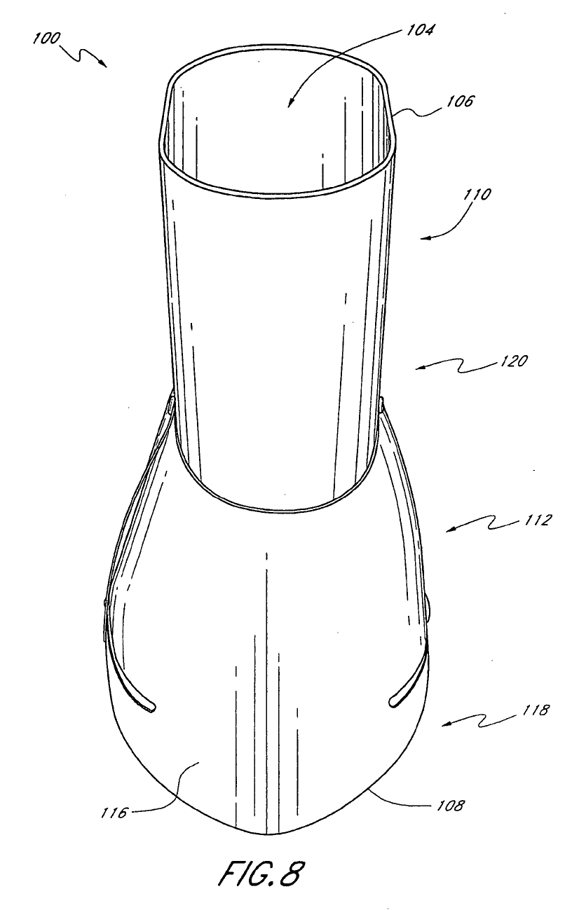

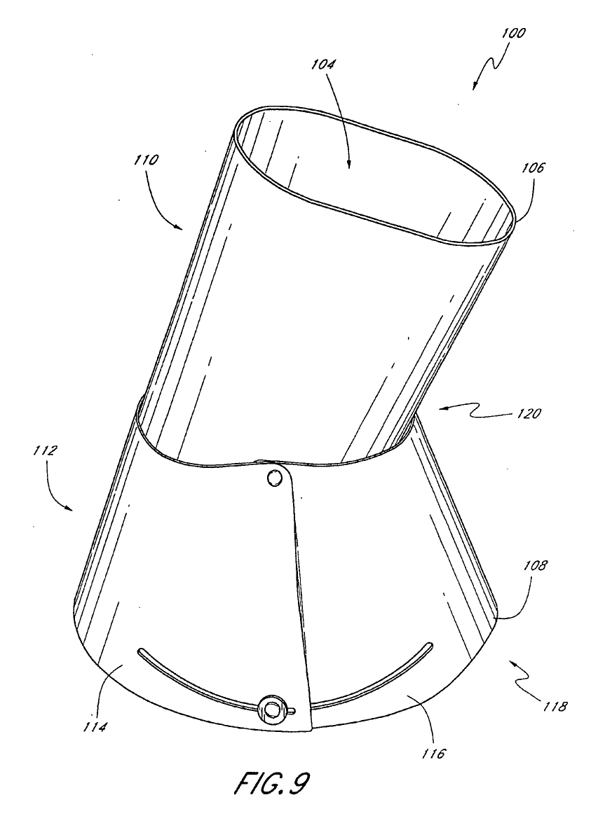

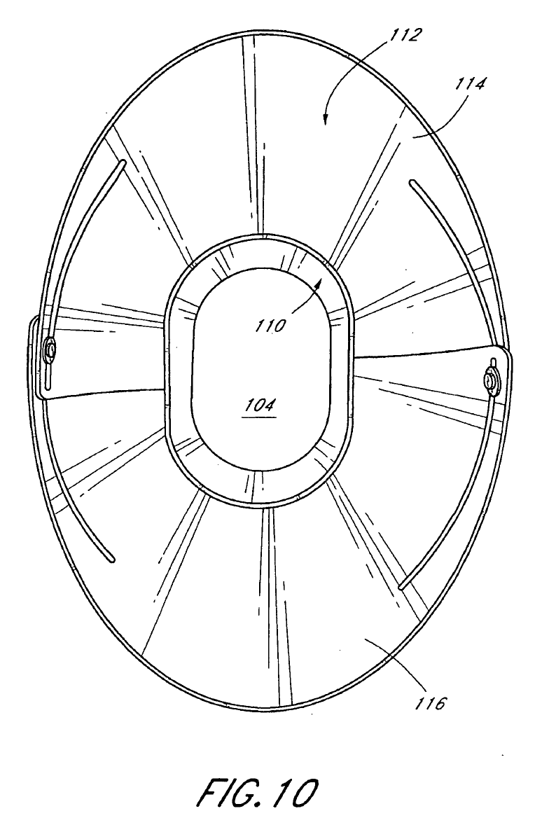

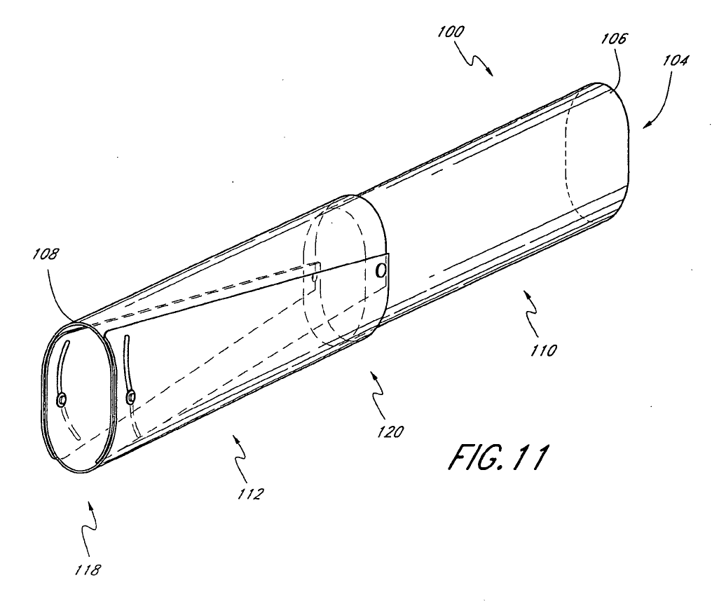

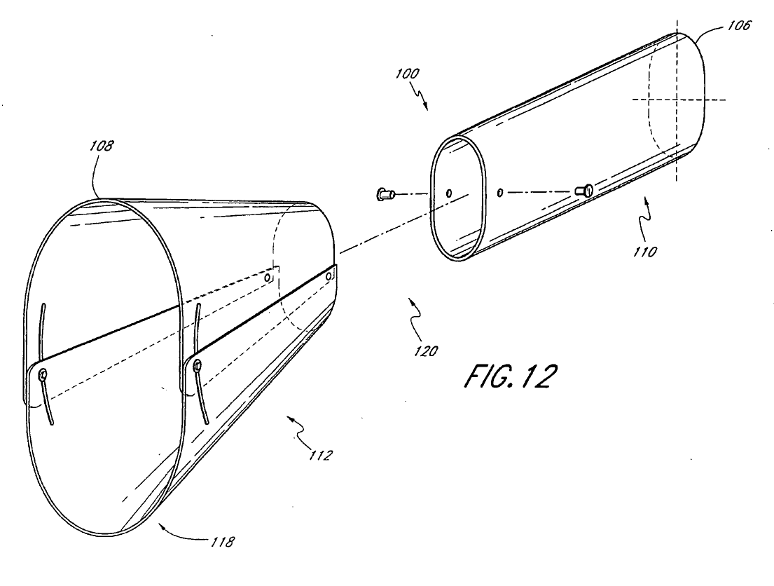

With reference to FIGS. 7-12, another embodiment of an access device 100 comprises an elongate body 102 defining a passage 104 and having a proximal end 106 and a distal end 108. The elongate body 102 has a proximal portion 110 and a distal portion 112. The proximal portion 110 has an oblong or generally oval shaped cross section in one embodiment. The term “oblong” is used in its ordinary sense (i.e., having an elongated form) and is a broad term and it includes a structure having a dimension, especially one of two perpendicular dimensions, such as, for example, width or length, that is greater than another and includes shapes such as rectangles, ovals, ellipses, triangles, diamonds, trapezoids, parabolas, and other elongated shapes having straight or curved sides. The term “oval” is used in its ordinary sense (i.e., egg like or elliptical) and is a broad term and includes oblong shapes having curved portions.

The proximal portion 110 comprises an oblong, generally oval shaped cross section over the elongated portion. It will be apparent to those of skill in the art that the cross section can be of any suitable oblong shape. The proximal portion 110 can be any desired size. The proximal portion 110 can have a cross-sectional area that varies from one end of the proximal portion to another end. For example, the cross-sectional area of the proximal portion can increase or decrease along the length of the proximal portion 110. Preferably, the proximal portion 110 is sized to provide sufficient space for inserting multiple surgical instruments through the elongate body 102 to the surgical location. The distal portion 112 preferably is expandable and comprises first and second overlapping skirt members 114, 116. The degree of expansion of the distal portion 112 is determined by an amount of overlap between the first skirt member 114 and the second skirt member 116 in one embodiment.

The elongate body 102 of the access device 100 has a first location 118 distal of a second location 120. The elongate body 102 preferably is capable of having a configuration when inserted within the patient wherein the cross-sectional area of the passage 104 at the first location 118 is greater than the cross-sectional area of the passage 104 at the second location 120. The passage 104 preferably is capable of having an oblong shaped cross section between the second location 120 and the proximal end 106. In some embodiments the passage 104 preferably is capable of having a generally elliptical cross section between the second location 120 and the proximal end 106. Additionally, the passage 104 preferably is capable of having a non-circular cross section between the second location 120 and the proximal end 106. Additionally, in some embodiments, the cross section of the passage 104 can be symmetrical about a first axis and a second axis, the first axis being generally normal to the second axis.

In another embodiment, an access device comprises an elongate body defining a passage and having a proximal end and a distal end. The elongate body can be a unitary structure and can have a generally uniform cross section from the proximal end to the distal end. In one embodiment, the elongate body preferably has an oblong or generally oval shaped cross section along the entire length of the elongate body. The passage can have a generally elliptical cross section between the proximal end and the distal end. The elongate body preferably has a relatively fixed cross-sectional area along its entire length. In one embodiment, the elongate body is capable of having a configuration when inserted within the patient wherein the cross-sectional area of the passage at a first location is equal to the cross-sectional area of the passage at a second location. The passage preferably is capable of having an oblong shaped cross section between the first and second locations. The cross section of the passage can be of any suitable oblong shape and the elongate body can be any desired size. Preferably, the elongate body is sized to provide sufficient space for inserting multiple surgical instruments sequentially or simultaneously through the elongate body to the surgical location.

In one embodiment, the access device has a uniform, generally oblong shaped cross section and is sized or configured to approach, dock on, or provide access to, anatomical structures. The access device preferably is configured to approach the spine from a posterior position or from a postero-lateral position. A distal portion of the access device can be configured to dock on, or provide access to, posterior portions of the spine for performing spinal procedures, such as, for example, fixation, fusion, or any other procedure described herein. In one embodiment, the distal portion of the access device has a uniform, generally oblong shaped cross section and is configured to dock on, or provide access to, generally posterior spinal structures. Generally posterior spinal structures can include, for example, one or more of the transverse process, the superior articular process, the inferior articular process, and the spinous process. In some embodiments, the access device can have a contoured distal end to facilitate docking on one or more of the posterior spinal structures. Accordingly, in one embodiment, the access device has a uniform, generally oblong shaped cross section with a distal end sized, configured, or contoured to approach, dock on, or provide access to, spinal structures from a posterior or postero-lateral position.

Further details and features pertaining to access devices and systems are described in U.S. patent application Ser. No. 09/772,605, filed Jan. 30, 2001, application Ser. No. 09/906,463, filed Jul. 16, 2001, application Ser. No. 10/361,887, filed Feb. 10, 2003, application Ser. No. 10/280,489, filed Oct. 25, 2002, and application Ser. No. 10/678,744 filed Oct. 2, 2003, which are incorporated by reference in their entireties herein.

2. Dilators and Expander Devices

According to one application or procedure, an early stage involves determining a point in the skin of the patient at which to insert the access device 20. The access point preferably corresponds to a posterior-lateral aspect of the spine. Manual palpation and Anterior-Posterior (AP) fluoroscopy may be used to determine preferred or optimal locations for forming an incision in the skin of the patient. In one application, the access device 20 preferably is placed midway (in the cephcaudal direction) between the L4 through S1 vertebrae, centrally about 4-7 cm from the midline of the spine.

After the above-described location is determined, an incision is made at the location. A guide wire (not shown) is introduced under fluoroscopic guidance through the skin, fascia, and muscle to the approximate surgical site. In one advantageous technique, an expandable dilator is used to increase the size of a passage extending beneath the incision. Various expandable dilators that can be used alone or in combination with a guide pin of guidewire are described below in Section III. In another technique, a series of dilators is used to sequentially expand the incision to the desired width, about 23 mm in one procedure, preferably minimizing damage to the structure of surrounding tissue and muscles. A first dilator can be placed over the guide wire to expand the opening. The guide wire may then be removed. A second dilator, slightly larger than the first dilator, is placed over the first dilator to expand the opening further. Once the second dilator is in place, the first dilator may be removed. This process of (1) introducing a next-larger-sized dilator coaxially over the previous dilator and (2) optionally removing the previous dilator(s) when the next-larger-sized dilator is in place continues until an opening of the desired size is created in the skin, muscle, and subcutaneous tissue. According to one application, the desired opening size is about 23 mm. Other dimensions of the opening, e.g., about 20 mm, about 27 mm, about 30 mm, etc., are also useful with this apparatus in connection with spinal surgery, and still other dimensions are contemplated.

FIG. 13 shows that following placement of a dilator 120, which is the largest dilator in the above-described dilation process, the access device 20 is introduced in its reduced profile configuration and positioned over the dilator 120. The dilator 120 is subsequently removed from the patient, and the access device 20 remains in position.

Once positioned in the patient, the access device 20 may be enlarged to provide a passage for the insertion of various surgical instruments and to provide an enlarged space for performing the procedures described herein. As described above, the access device may achieve the enlargement in several ways. In one embodiment, a distal portion of the access device may be enlarged, and a proximal portion may maintain a constant diameter. The relative lengths of the proximal portion 22 and the skirt portion 24 may be adjusted to vary the overall expansion of the access device 20. Alternatively, such expansion may extend along the entire length of the access device 20. In one application, the access device 20 may be expanded by removing a suture 35 and tearing the outer sleeve 32 surrounding the access device 20, and subsequently allowing the skirt portion 24 to resiliently expand towards its fully expanded configuration as (illustrated in FIG. 4) to create an enlarged surgical space from the L4 to the S1 vertebrae. The resisting force exerted on the skirt portion 24 may result in the skirt portion 24 assuming the intermediate configuration illustrated in FIG. 3. Under many circumstances, the space created by the skirt portion 24 in the intermediate configuration is a sufficiently large working space to perform the procedure described herein. Once the skirt portion 24 has expanded, the rigidity and resilient characteristics of the skirt portion 24 preferably allow the access device 20 to resist closing to the reduced profile configuration of FIG. 2 and to at least temporarily resist being expelled from the incision. These characteristics create a stable configuration for the access device 20 to remain in position in the body, supported by the surrounding tissue. It is understood that additional support may be needed, especially if an endoscope is added.

According to one embodiment of a procedure, the access device 20 may be further enlarged at the skirt portion 24 using an expander apparatus to create a surgical access space. An expander apparatus useful for enlarging the access device has a reduced profile configuration and an enlarged configuration. The expander apparatus is inserted into the access device in the reduced profile configuration, and subsequently expanded to the enlarged configuration. The expansion of the expander apparatus also causes the access device to be expanded to the enlarged configuration. In some embodiments, the expander apparatus may increase the diameter of the access device along substantially its entire length in a generally conical configuration. In other embodiments, the expander apparatus expands only a distal portion of the access device, allowing a proximal portion to maintain a relatively constant diameter.

In addition to expanding the access device, in some embodiments the expander apparatus may also be used to position the distal portion of the access device at the desired location for the surgical procedure. The expander can engage an interior wall of the access device to move the access device to the desired location. For embodiments in which the distal portion of the access device is relatively movable with respect to the proximal portion, the expander apparatus is useful to position the distal portion without substantially disturbing the proximal portion.

In some procedures, an expander apparatus is used to further expand the skirt portion 24 towards the enlarged configuration (illustrated in FIG. 4). The expander apparatus is inserted into the access device, and typically has two or more members that are movable to engage the interior wall of the skirt portion 24 and apply a force sufficient to further expand the skirt portion 24. FIGS. 14 and 15 show one embodiment of an expander apparatus 200 that has a first component 202 and a second component 204. The first component 202 and the second component 204 of the expander apparatus 200 are arranged in a tongs-like configuration and are pivotable about a pin 206. The first and second components 202 and 204 can be constructed of steel having a thickness of about 9.7 mm. Each of the first and second components 202 and 204 has a proximal handle portion 208 and a distal expander portion 210. Each proximal handle portion 208 has a finger grip 212 that may extend transversely from an axis, e.g., a longitudinal axis 214, of the apparatus 200. The proximal handle portion 208 may further include a stop element, such as flange 216, theat extends transversely from the longitudinal axis 214. The flange 216 preferably is dimensioned to engage the proximal end 25 of the access device 20 when the apparatus 200 is inserted a predetermined depth. This arrangement provides a visual and tactile indication of the proper depth for inserting the expander apparatus 200. In one embodiment, a dimension 218 from the flange 216 to the distal tip 220 is about 106 mm. The dimension 218 is determined by the length of the access device 20, which in turn is a function of the depth of the body structures beneath the skin surface at which the surgical procedure is to be performed. The distal portions 210 are each provided with an outer surface 222 for engaging the inside wall of the skirt portion 24. The outer surface 222 is a frusto-conical surface in one embodiment. The expander apparatus 200 has an unexpanded distal width 224 at the distal tip 220 that is about 18.5 mm in one embodiment.

In use, the finger grips 212 are approximated towards one another, as indicated by arrows A in FIG. 15, which causes the distal portions 210 to move to the enlarged configuration, as indicated by arrows B. The components 202 and 204 are also provided with a cooperating tab 226 and shoulder portion 228 which are configured for mutual engagement when the distal portions 210 are in the expanded configuration. In the illustrated embodiment, the expander apparatus 200 has an expanded distal width 230 that extends between the distal portions 210. The expanded distal width 230 can be about 65 mm or less, about as large as 83 mm or less, or any other suitable width. The tab 226 and shoulder portion 228 together limit the expansion of the expander apparatus 200 to prevent expansion of the skirt portion 24 of the access device 20 beyond its designed dimension, and to minimize trauma to the underlying tissue. Further features related to the expander apparatus are described in U.S. Pat. No. 6,652,553, issued Nov. 25, 2003, which is incorporated by reference in its entirety herein.

When the access device 20 is inserted into the patient and the Outer sleeve 32 is removed, the skirt portion 24 expands to a point where the outward resilient expansion of the skirt portion 24 is balanced by the force of the surrounding tissue. The surgical space defined by the access device 20 may be sufficient to perform any of a number of surgical procedures or combination of surgical procedures described herein. However, if it is desired to expand the access device 20 further, the expander apparatus 200, or a similar device, may be inserted into the access device 20 in the reduced profile configuration until the shoulder portions 216 are in approximation with the proximal end 25 of the skirt portion 24 of the access device 20, as shown in FIG. 16.

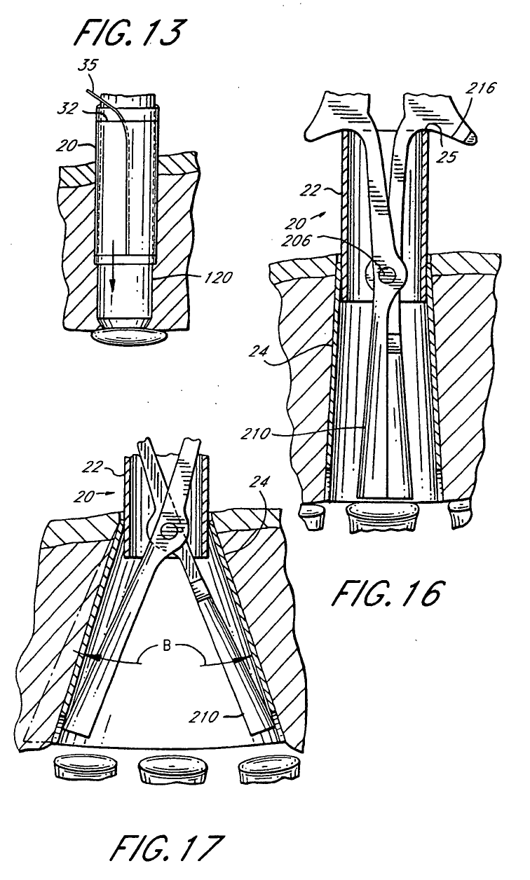

FIG. 16 shows the expander apparatus 200 inserted in the access device 20 in the reduced profiled configuration. Expansion of the expander apparatus 200 is achieved by approximating the handle portions 212 (not shown in FIG. 16), which causes the distal portions 210 of the expander apparatus 200 to move to a spaced apart configuration. As the distal portions 210 move apart and contact the inner wall of the skirt portion 24, the rivet 44 is allowed to slide within the slots 46 and 48 of the skirt portion 24, thus permitting the skirt portion 24 to expand. When the distal portions 210 reach the maximum expansion of the skirt portion 24 (illustrated by a dashed line in FIG. 17), the tab 226 and shoulder portion 228 of the expander apparatus 200 come into engagement to prevent further expansion of the tongs-like portions (as illustrated in FIG. 15). Alternatively, the access device 20 may be expanded with another device that can selectively have a reduced profile configuration and an expanded configuration, e.g., a balloon or similar device.

An optional step in the procedure is to adjust the location of the distal portion of the access device 20 relative to the body structures to be operated on. For example, the expander apparatus 200 may also be used to engage the inner wall of the skirt portion 24 of the access device 20 in order to move the skirt portion 24 of the access device 20 to the desired location. For an embodiment in which the skirt portion 24 of the access device 20 is relatively movable relative to the proximal portion, e.g. by use of the rivet 30, the expander apparatus 200 is useful to position the skirt portion 24 without substantially disturbing the proximal portion 22 or the tissues closer to the skin surface of the patient. As will be described below, the ability to move the distal end portion, e.g., the skirt portion 24, without disturbing the proximal portion is especially beneficial when an additional apparatus is mounted relative to the proximal portion of the access device, as described below.

B. Systems and Devices for Stabilization and Visualization

Some procedures can be conducted through the access device 20 without any additional peripheral components being connected thereto. In other procedures it may be beneficial to provide at least one of a support device and a viewing element. As discussed more fully below, support devices can be advantageously employed to provide support to peripheral equipment and to surgical tools of various types. Various embodiments of support devices and viewing elements are discussed herein below.

1. Support Devices

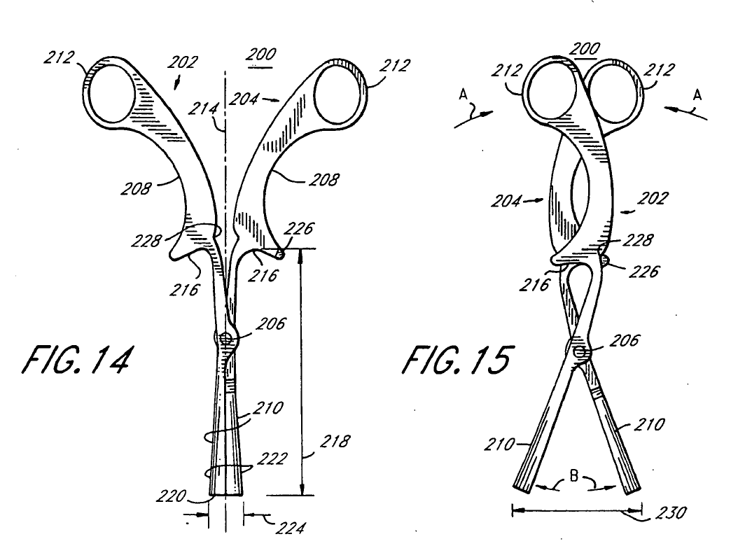

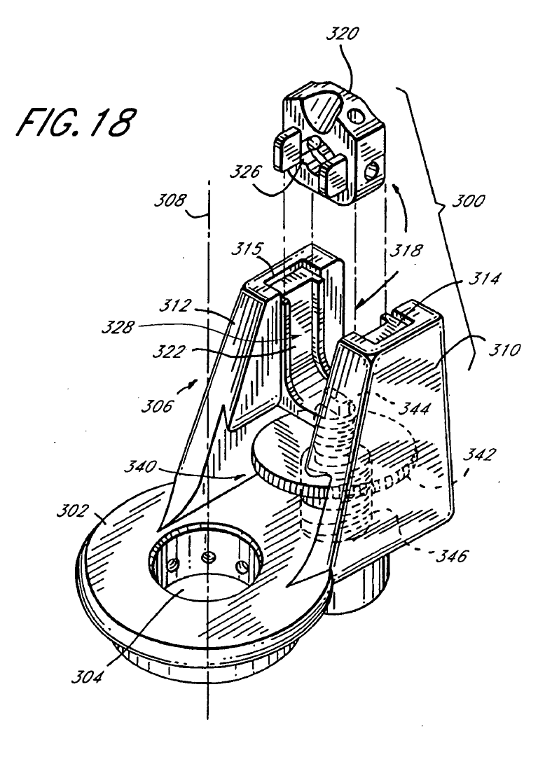

One type of support device that can be coupled with the access device 20 is a device that supports a viewing element. In one embodiment, an endoscope mount platform 300 and indexing arm 400 support an endoscope 500 on the proximal end 25 of the access device 20 for remotely viewing the surgical procedure, as illustrated in FIGS. 18-21. The endoscope mount platform 300 may also provide several other functions during the surgical procedure. The endoscope mount platform 300 preferably includes a base 302 that extends laterally from a central opening 304 in a generally ring-shaped configuration. In one application, the physician views the procedure primarily by observing a monitor, when inserting surgical instruments into the central opening 304. The base 302 advantageously enables the physician by providing a visual indicator (in that it may be observable in the physician's peripheral vision) as well as tactile feedback as instruments are lowered towards the central opening 304 and into the access device 20.

The endoscope mount platform 300 preferably has a guide portion 306 at a location off-set from the central opening 304 that extends substantially parallel to a longitudinal axis 308. The base 302 can be molded as one piece with the guide portion 306. The base 302 and guide portion 306 may be constructed with a suitable polymer, such as, for example, polyetheretherketone (PEEK).

The guide portion 306 includes a first upright member 310 that extends upward from the base 302 and a second upright member 312 that extends upward from the base 302. In one embodiment, the upright members 310, 312 each have a respective vertical grooves 314 and 315 that can slidably receive an endoscopic mount assembly 318.

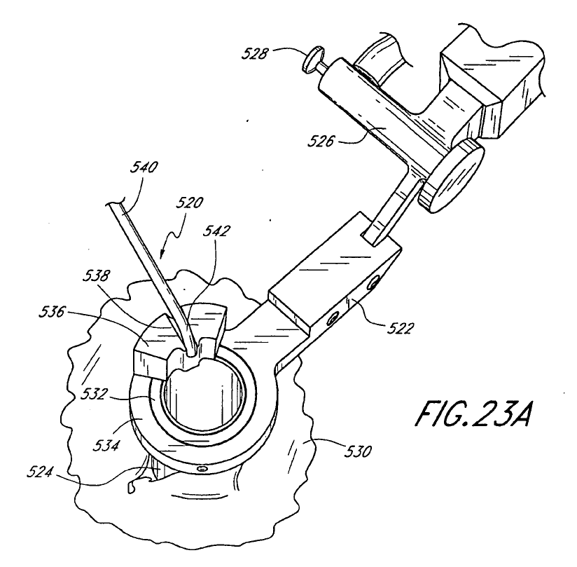

The endoscope 500 (not shown in FIG. 18) can be movably mounted to the endoscope mount platform 300 with the endoscope mount assembly 318 in one embodiment. The endoscope mount assembly 318 includes an endoscope mount 320 and a saddle unit 322. The saddle unit 322 is slidably mounted within the grooves 314 and 315 in the upright members 310 and 312. The endoscope mount 320 receives the endoscope 500 through a bore 326 which passes through the endoscope mount 320. Part of the endoscope 500 may extend through the access device 20 substantially parallel to longitudinal axis 308 into the patient's body 130, as shown in FIG. 25.

The endoscope mount 320 is removably positioned in a recess 328 defined in the substantially “U”-shaped saddle unit 322. In one embodiment, the saddle unit 322 is selectively movable in a direction parallel to the longitudinal axis 308 in order to position the endoscope 500 at the desired height within the access device 20. The movement or the endoscope 500 by way of the saddle unit 322 also advantageously enables the physician to increase visualization of a particular portion of the surgical space defined by the access device, e.g., by way of a zoom feature, as required for a given procedure or a step of a procedure.

In one embodiment, an elevation adjustment mechanism 340, which may be a screw mechanism, is positioned on the base 302 between the upright members 310 and 312. The elevation adjustment mechanism 340 can be used to selectively move a viewing element, e.g., the endoscope 500 by way of the saddle unit 322. In one embodiment, the elevation adjustment mechanism 340 comprises a thumb wheel 342 and a spindle 344. The thumb wheel 343 is rotatably mounted in a bore in the base 302. The thumb wheel 342 has an external thread 346 received in a cooperating thread in the base 302. The spindle 344 is mounted for movement substantially parallel to the central axis 308. The spindle 344 preferably has a first end received in a rectangular opening in the saddle unit 322, which inhibits rotational movement of the spindle 344. The second end of the spindle 344 has an external thread that cooperates with an internal thread formed in a bore within the thumb wheel 342. Rotation of the thumb wheel 342 relative to the spindle 344, causes relative axial movement of the spindle unit 344 along with the saddle unit 322. Further details and features related to endoscope mount platforms are described in U.S. Pat. No. 6,361,488, issued Mar. 26, 2002; U.S. Pat. No. 6,530,880, issued Mar. 11, 2003, and U.S. patent application Ser. No. 09/940,402, filed Aug. 27, 2001, published as Publication No. 2003/0040656 on Feb. 27, 2003, which are incorporated by reference in their entireties herein.

FIGS. 19-21 show that the endoscope mount platform 300 is mountable to the support arm 400 in one embodiment. The support arm 400, in turn, preferably is mountable to a mechanical support, such as mechanical support arm A, discussed above in connection with FIG. 1. The support arm 400 preferably rests on, or is otherwise coupled to, the proximal end 25 of the access device 20. In one embodiment, the support arm 400 is coupled with an indexing collar 420, which is configured to be received in the central opening 304 of the base 302 of endoscope mount platform 300. The indexing collar 420 is substantially toroidal in section and has an outer peripheral wall surface 422, an inner wall surface 424, and a wall thickness 426 that is the distance between the wall surfaces 422, 424. The indexing collar 420 further includes a flange 428, which supports the indexing collar 420 on the support arm 400.

In one embodiment, a plurality of collars 420 may be provided to make the surgical system 10 modular in that different access devices 20 may be used with a single endoscope mount platform 300. For example, access devices 20 of different dimensions may be supported by providing indexing collars 420 to accommodate each access device size while using a single endoscope mount platform 300. The central opening 304 of the endoscope mount platform 300 can have a constant dimension, e.g., a diameter of about 32.6 mm. An appropriate indexing collar 420 is selected, e.g., one that is appropriately sized to support a selected access device 20. Thus, the outer wall 422 and the outer diameter 430 are unchanged between different indexing collars 420, although the inner wall 424 and the inner, diameter 432 vary to accommodate differently sized access devices 20.

The indexing collar 420 can be mounted to the proximal portion of the access device 20 to allow angular movement of the endoscope mount platform 300 with respect thereto about the longitudinal axis 308 (as indicated by an arrow C in FIG. 19). The outer wall 422 of the index collar 420 includes a plurality of hemispherical recesses 450 that can receive one or more ball plungers 350 on the endoscope mount platform 300 (indicated in dashed line). This arrangement permits the endoscope mount platform 300, along with the endoscope 500, to be fixed in a plurality of discrete angular positions.

Further details and features related to support arms and indexing collars are described in U.S. Pat. No. 6,361,488, issued Mar. 26, 2002, U.S. Pat. No. 6,530,880 issued Mar. 11, 2003, and application Ser. No. 09/940,402 filed Aug. 27, 2001, published as Publication No. 2003/0040656 on Feb. 27, 2003, which are incorporated by reference in their entireties herein.

2. Viewing Elements

As discussed above, a variety of viewing elements and visualization techniques are embodied in variations of the surgical system 10. One viewing element that is provided in one embodiment is an endoscope.

FIG. 22 shows one embodiment of the endoscope 500 that has an elongated configuration that extends into the access device 20 in order to enable viewing of the surgical site. In particular, the endoscope 500 has an elongated rod portion 502 and a body portion 504. The rod portion 502 extends generally perpendicularly from the body portion 504. In one embodiment, the rod portion 502 of endoscope 500 has a diameter of about 4 mm and a length of about 106 mm. Body portion 504 may define a tubular portion 506 configured to be slidably received in the bore 326 of endoscope mount 320 as indicated by an arrow D. The slidable mounting of the endoscope 500 on the endoscope mount platform 300 permits the endoscope 500 to adjust to access device configurations that have different diameters. Additional mobility of the endoscope 500 in viewing the surgical site may be provided by rotating the endoscope mount platform 300 about the central axis 308 (as indicated by arrow C in FIG. 19).

The rod portion 502 supports an optical portion (not shown) at a distal end 508 thereof. In one embodiment, the rod portion 502 defines a field of view of about 105 degrees and a direction of view 511 of about 25-30 degrees. An eyepiece 512 preferably is positioned at an end portion of the body portion 504. A suitable camera (not shown) preferably is attached to the endoscope 500 adjacent the eyepiece 512 with a standard coupler unit. A light post 510 can supply illumination to the surgical site at the distal end portion 508. A preferred camera for use in the system and procedures described herein is a three chip unit that provides greater resolution to the viewed image than a single chip device.

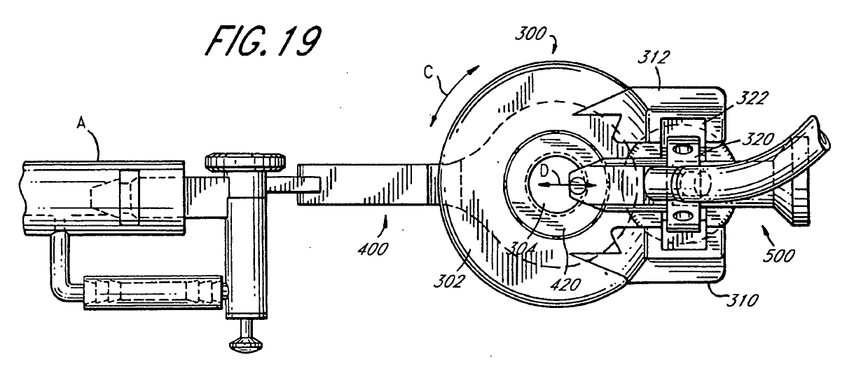

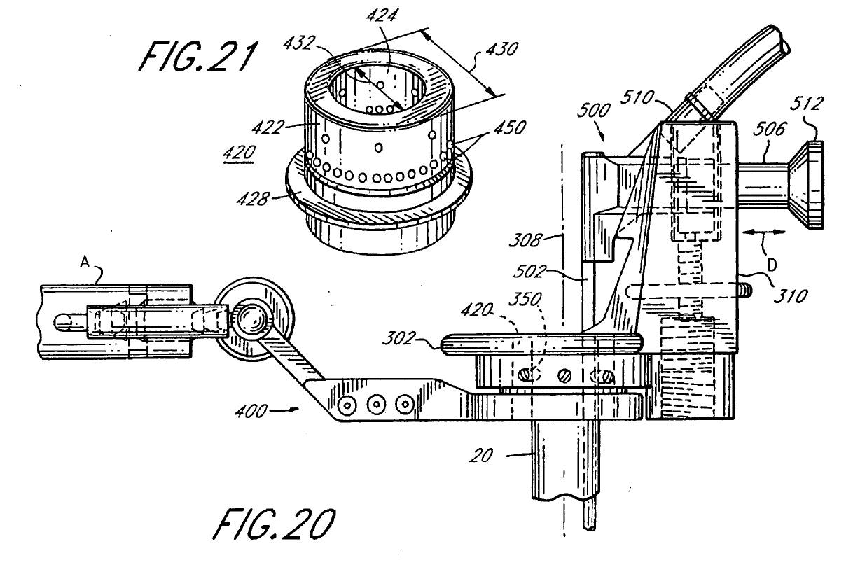

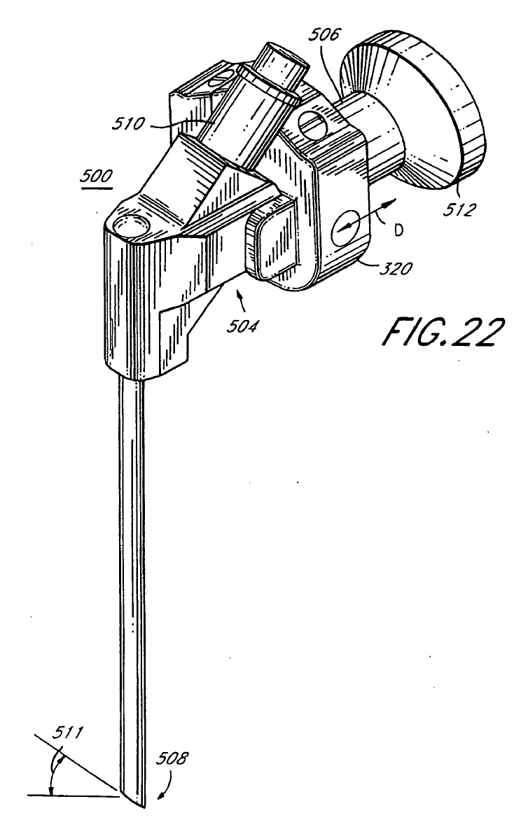

FIGS. 23A, 23B, 23C, 24A, 24B, and 24C illustrate other embodiments of support devices and viewing elements. FIGS. 23A, 23B, and 23C illustrate one embodiment of a lighting element 520 coupled with a support arm 522 compatible with an access device 524 having a proximal portion with a generally circular cross section. In other embodiments, support arms can be configured to be coupled with access devices having proximal portions with generally oblong or oval cross sections.

The support arm 522 preferably is coupled with the access device 524 to provide support for the access device 524 during a procedure. As shown in FIGS. 23A, 23B, and 23C, the support arm 522 comprises a pneumatic element 526 for maintaining the support arm 522 in a desired position. Depressing a button 528 coupled with a valve of the pneumatic element 526 releases pressure and allows the support arm 522 and access device 524 to be moved relative the patient 530. Releasing the button 528 of the pneumatic element 526 increases pressure and maintains the access device 524 and support arm 522 in the desired position. The support arm 522, as shown, is configured for use with a mechanical arm using a suction, or a vacuum to maintain the access device in a desired location. One of skill in the art will recognize that various other support arms and mechanical arms can be used. For example, commercially available mechanical arms having clamping mechanisms can be used as well as suction or pressure based arms.

The support arm 522 can comprise an inner ring portion 532 and an outer ring portion 534 for surrounding the access device 524 at its proximal end. In the illustrated embodiment, the inner and outer ring portions 532, 534 are fixed relative each other. In other embodiments the inner and outer ring portions 532, 534 can move relative each other. The support arm 522 preferably comprises a lighting element support portion 536. In the illustrated embodiment, the lighting element support portion 536 extends above upper surfaces of the inner and outer ring portions 532, 534. The lighting element support portion 536 can extend from the inner ring portion 532, the outer ring portion 534, or both. The lighting element support portion 536 can have a notch or groove 538 for receiving and supporting the lighting element 520. Additionally, the lighting element support portion 536 can have one or more prongs extending at least partially over the lighting element 520 to hold it in place.

In the illustrated embodiment, the lighting element 520 has an elongated proximal portion 540 and a curved distal portion 542. The proximal portion 540 of the lighting element 520 preferably is coupled with a light source (not shown). The curved distal portion of the lighting element 520 in one embodiment extends only a short distance into the access device and is configured to direct light from the light source down into the access device 524. In another embodiment, the lighting element 520 can be provided such that it does not extend into the access device. In such an embodiment, the right portions 532 and 534 only partially surround the proximal end of the access device 524. Providing a lighting element 520 for use with the access device 524 preferably allows a user to see down into the access device 524 to view a surgical location. Accordingly, use of a lighting element 520 in some cases, enables the user to perform a procedure, in whole or in part, without the use of an endoscope. In one embodiment, the lighting element 520 enables a surgeon to perform the procedure with the use of microscopes or loupes.

FIGS. 24A, 24B, and 24C illustrate other embodiments of visualization elements. As shown in FIG. 24A, a lighting element 560 comprises a support member 562, an access device insert 564, and fiber optic elements 566. The support member 562 has a proximal end 568, a central portion 570, and a distal end 572. The proximal end 568 preferably has a coupling portion 574 for coupling the support member 562 to a support arm or other support system (not shown). The central portion 570 preferably is coupled with the fiber optic elements 566 to provide support there to. The distal end 572 preferably is coupled with the access device insert 564.

In the illustrated embodiment, the access device insert 564 is configured to be inserted in an access device having a proximal portion with a generally circular cross section. The access device insert 564 is coupled with the fiber optic elements 566. The fiber optic elements 566 extend down into the access device insert 564 so that the ends of the fiber optic elements 566 can direct light down inside an access device along side portions there of.

FIGS. 24B and 24C illustrate other embodiments of visualization elements similar to the embodiment described with reference to FIG. 24A. In the illustrated embodiments, the access device inserts 564 are configured to be inserted into access devices having proximal portions with generally oblong, or oval, cross sections. As shown in FIG. 24B, the access device insert 564 has a generally oblong or oval shaped cross section. The access device insert 564 is coupled with the fiber optic elements 566 along a longer side surface of the access device insert 564. As shown in FIG. 24C, the access device insert 564 has a generally oblong or oval shaped cross section. The access device insert 564 is coupled with the fiber optic elements 566 along a shorter side surface of the access device insert 564. Use of an illumination element with an expandable access device having an oblong shaped proximal section, in some cases, allows a doctor to perform procedures that would be difficult to perform using an endoscope. Increased visualization of the surgical location through the access device can simplify some procedures. For example, decompression of the contra-lateral side can be achieved more easily in some cases without the use of an endoscope.

C. Apparatuses and Methods for Performing Spinal Procedures

The surgical assembly 10 described above can be deployed to perform a wide variety of surgical procedures on the spine. In many cases, the procedures are facilitated by inserting the access device and configuring it to provide greater access to a surgical location, as discussed above and by mounting the support arm 400 and the endoscope mount platform 300 on the proximal portion, e.g., on the proximal end 25, of the access device 20 (FIGS. 1 and 22). As discussed above, visualization of the surgical location is enhanced by mounting a viewing element, such as the endoscope 500, on the endoscope mount platform 300. Having established increased access to and visualization of the surgical location, a number of procedures may be effectively performed.

Generally, the procedures involve inserting one or more surgical instruments into the access device 20 to manipulate or act on the body structures that are located at least partially within the operative space defined by the expanded portion of the access device 20. FIG. 25 shows that in one method, the skirt portion 24 of access device 20 at least partially defines a surgical site or operative space 90 in which the surgical procedures described herein may be performed. Depending upon the overlap of the skirt portion, the skirt portion may define a surface which is continuous about the perimeter or which is discontinuous, having one or more gaps where the material of the skirt portion does not overlap.

One procedure performable through the access device 20, described in greater detail below, is a two-level spinal fusion and fixation. Surgical instruments inserted into the access device may be used for debridement and decortication. In particular, the soft tissue, such as fat and muscle, covering the vertebrae may be removed in order to allow the physician to visually identify the various “landmarks,” or vertebral structures, which enable the physician to determine the location for attaching a fastener, such a fastener 600, discussed below, or other procedures, as will be described herein. Enabling visual identification of the vertebral structures enables the physician to perform the procedure while viewing the surgical area through the endoscope, microscope, loupes, or other viewing element, or in a conventional, open manner.

Tissue debridement and decortication of bone are completed using one or more of a debrider blades, a bipolar sheath, a high speed burr, and any other conventional manual instrument. The debrider blades are used to excise, remove and aspirate the soft tissue. The bipolar sheath is used to achieve hemostasis through spot and bulk tissue coagulation. Additional features of debrider blades and bipolar sheaths are described in U.S. Pat. No. 6,193,715, assigned to Medical Scientific, Inc., which is incorporated by reference in its entirety herein. The high speed burr and conventional manual instruments are also used to continue to expose the structure of the vertebrae.

1. Fixation Systems and Devices

Having increased visualization of the pertinent anatomical structure, various procedures may be carried out on the structures. In one procedure, one or more fasteners are attached to adjacent vertebrae V. As discussed in more detail below, the fasteners can be used to provide temporary or permanent fixation and to provide dynamic stabilization of the vertebrae V. These procedures may combined with other procedures, such as procedures employing other types of implant, e.g., procedures employing fusion devices, prosthetic disc components, or other suitable implants. In some procedures, fasteners are attached to the vertebrae before or after fusion devices are inserted between the vertebrae V. Fusion systems and devices are discussed further below.

In one application, the desired location and orientation of the fastener is determined before the fastener is applied to the vertebra. The desired location and orientation of the fastener may be determined in any suitable manner. For example, the pedicle entry point of the L5 vertebrae may be located by identifying visual landmarks alone or in combination with lateral and A/P fluoroscopy, as is known in the art. With continued reference to FIG. 25, an entry point 92 into the vertebra V is prepared. In procedure, the entry point 92 may be prepared with an awl 550. The entry point 92 corresponds to the pedicle in one procedure. The entry point 92 may be prepared in any suitable manner, e.g. employing a bone probe, a tap, and a sounder to create and verify the integrity of the prepared vertebra. The sounder, as is known in the art, determines whether the hole that is made is surrounded by bone on all sides, and can be used to confirm that there has been no perforation of the pedicle wall.

After the hole in the pedicle beneath the entry point 92 is prepared, a fastener may be advanced into the hole. Prior to advancing the fastener, or at any other point during the procedure, it may be desirable to adjust the location of the distal portion of the access device 20. The distal portion of the access device 20 may be adjusted by inserting the expander apparatus 200 into the access device 20, expanding the distal portions 210, and contacting the inner wall of the skirt portion 24 to move the skirt portion 24 to the desired location. This step may be performed while the endoscope 500 is positioned within the access device 20, and without substantially disturbing the location of the proximal portion of the access device 20 to which the endoscope mount platform 300 may be attached.

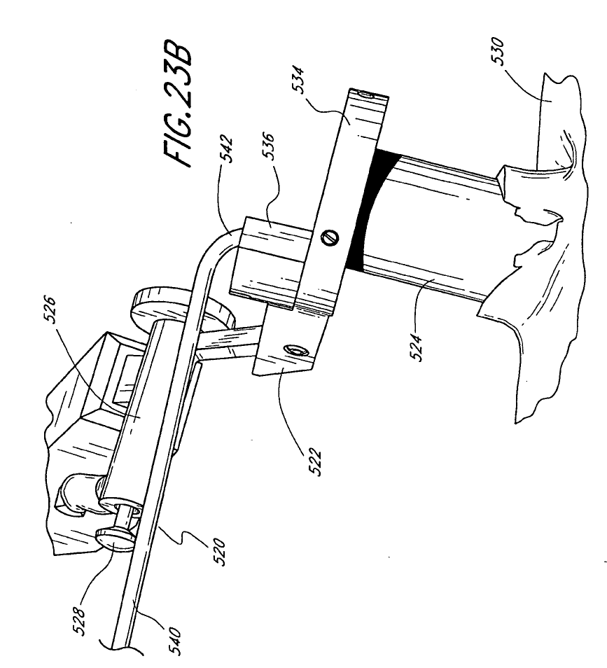

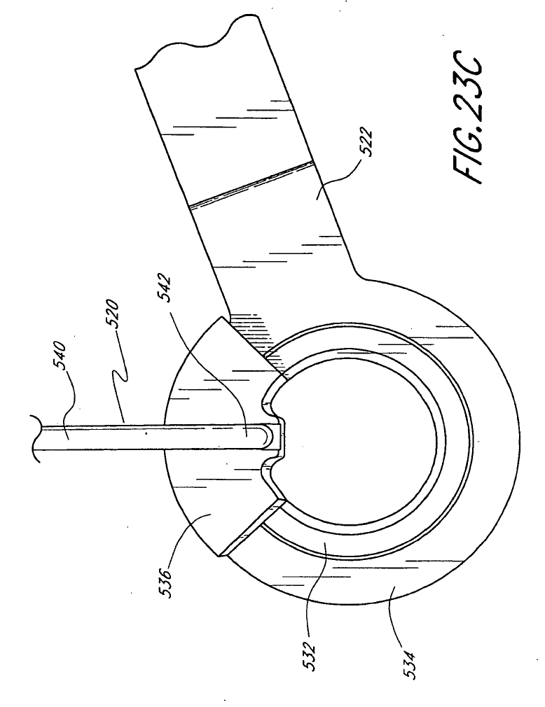

FIGS. 26-27 illustrate one embodiment of a fastener 600 that is particularly applicable in procedures involving fixation. The fastener 600 preferably includes a screw portion 602, a housing 604, a spacer member 606, a biasing member 608, and a clamping member, such as a cap screw 610. The screw portion 602 has a distal threaded portion 612 and a proximal, substantially spherical joint portion 614. The threaded portion 612 is inserted into the hole that extends away from the entry point 92 into the vertebrae, as will be described below. The substantially spherical joint portion 614 is received in a substantially annular, partly spherical recess 616 in the housing 604 in a ball and socket joint relationship (see also FIG. 29).

As illustrated in FIG. 27, the fastener 600 is assembled by inserting the screw portion 602 into a bore in a passage 618 in the housing 604 until the joint portion 614 engages the annular recess 616. The screw portion 602 is retained in the housing 604 by the spacer member 606 and by the biasing member 608. The biasing member 608 provides a biasing force to drive the spacer member 606 into frictional engagement with the joint portion 614 of the screw member 602 and the annular recess 616 of the housing 604. The biasing provided by the biasing member 602 frictionally maintains the relative positions of the housing 604 with respect to the screw portion 602. The biasing member 608 preferably is selected such that biasing force prevents unrestricted movement of the housing 604 relative to the screw portion 602. However, in some embodiments the biasing force is insufficient to resist the application of force by a physician to move the housing 604 relative to the screw portion 602. In other words, this biasing force is strong enough maintain the housing 604 stationary relative to the screw portion 602, but this force may be overcome by the physician to reorient the housing 604 with respect to the screw member 602, as will be described below.

In the illustrated embodiment, the biasing member 608 is a resilient ring having a gap 620, which permits the biasing member 608 to radially contract and expand. FIG. 27(a) illustrates that the biasing member 608 may have an arched shape, when viewed end-on. The arched shape of the spring member 608 provides the biasing force, as will be described below. The spacer member 606 and the biasing member 608 are inserted into the housing 604 by radially compressing the biasing member into an annular groove 622 in the spacer member 606. The spacer member 606 and the biasing member 608 are slid into the passage 618 until the distal surface of the spacer member 606 engages the joint portion 614 of the screw portion 602, and the biasing member 608 expands radially into the alnular groove 622 in the housing 604. The annular groove 622 in the housing 604 has a dimension 623 that is smaller than the uncompressed height of the arched shape of the biasing member 608. When the biasing member 608 is inserted in the annular groove 620, the biasing member 608 is flattened against its normal bias, thereby exerting the biasing force to the spacer member 606. It is understood that similar biasing members, such as coiled springs, belleville washers, or the like may be used to supply the biasing force described herein.

The spacer member 606 is provided with a longitudinal bore 626, which provides access to a hexagonal recess 628 in the proximal end of the joint portion 614 of the screw member 602. The proximal portion of the housing 604 includes a pair of upright members 630 and 631 that are separated by substantially “U”-shaped grooves 632. A recess for receiving elongated member 650 is defined by the pair of grooves 632 between upright members 630 and 631. Elongated member 650 preferably is configured to be placed distally into the housing 604 in an orientation substantially transverse to the longitudinal axis of the housing 604, as will be described below. The inner walls of he upright members 630 and 631 are provided with threads 634 for attachment of the cap screw 610 by threads 613 therein.

Additional features of the fastener 600 are also described in U.S. patent application Ser. No. 10/075,668, filed Feb. 13, 2002, published as U.S. Application Publication No. 2003/0153911A1 on Aug. 14, 2003, and application Ser. No. 10/087,489, filed Mar. 1, 2002, published as U.S. Application Publication No. 2003/0167058A1 oil Sep. 4, 2003, which are incorporated by reference in their entireties herein.

According to one application, the fastener 600 is inserted into the access device 20 and guided to the prepared hole at the entry point 92 in the vertebrae. The fastener 600 preferably is simultaneously supported and advanced into the hole so that the fastener 600 is secured in the in the hole beneath the entry point 92. In the illustrated embodiment the fastener 600 is supported and attached to the bone by an endoscopic screwdriver apparatus 660, illustrated in FIGS. 28-29. The screwdriver 660 includes a proximal handle portion 662 (illustrated in dashed line), an elongated body portion 664, and a distal tool portion 666.

The distal tool portion 666, as illustrated in greater detail in FIG. 29 includes a substantially hexagonal outer periphery that is received in the substantially hexagonal recess 628 in the joint portion 614 of the screw member 602. A spring member at the distal tool portion 666 releasably engages the hexagonal recess 628 of the screw member 602 to support the fastener 600 during insertion and tightening. In the illustrated embodiment, a spring member 672 is configured to engage the side wall of the recess 628. More particularly, a channel or a groove is provided in the tip portion 666 for receiving the spring member 672. The channel or groove includes a medial longitudinal notch portion 676, a proximal, angled channel portion 678, and a distal substantially transverse channel portion 680. The spring member 672 is preferably manufactured from stainless steel and has a medial portion 682, proximal portion 684, and a transverse distal portion 686. The medial portion 682 is partially received in the longitudinal notch portion 676. The proximal portion 684 preferably is angled with respect to the medial portion 682 and is fixedly received in the angled channel portion 678. The transverse distal portion 686 preferably is slidably received in the transverse channel 680. The medial portion 682 of the spring member 672 is partially exposed from the distal tip portion 666 and normally is biased in a transverse outward direction with respect to the longitudinal axis (indicated by arrow E), in order to Supply bearing force against the wall of the recess 628. Alternatively, the distal tip portion of the screwdriver may be magnetized in order to hold the screw portion 602. Similarly, the distal tip portion may include a ball bearing or similar member which is normally biased in a radially outward direction to engage the interior wall of the recess 628 to secure the faster 600 to the screwdriver distal tip 666. Other means may be provided for temporarily but securely coupling the fastener 600 with the screwdriver distal tip 666.

The insertion of the fastener 600 into the prepared hole that extends into the vertebrae from the entry point 92 may be achieved by insertion of screwdriver 660 into access device 20 (indicated by arrow G). This procedure may be visualized by the use of the endoscope 500 in conjunction with fluoroscopy, or by way of any other suitable viewing element. The screw portion 602 is threadedly advanced by the endoscopic screwdriver 660 into the prepared hole that extends beneath the entry point 92 (indicated by arrow H). The endoscopic screwdriver 660 is subsequently separated from the fastener 600, by applying a force in the proximal direction, and thereby releasing the distal tip portion 666 from the hexagonal recess 628 (e.g., causing the transverse distal portion 686 of the spring member 672 to slide within the transverse recess 680 against the bias, indicated by arrow F), and removing the screwdriver 660 from the access device 20. An alternative method may use a guidewire, which is fixed in the hole beneath the entry point 92, and a cannulated screw which has an internal lumen and is guided over the guidewire into the hole beneath the entry point 92. Where a guidewire system is used, the screwdriver also would be cannulated so that the screwdriver would fit over the guidewire.

For a two-level fixation, it may be necessary to prepare several holes and attach several fasteners 600. Preferably, the access device 20 is sized to provide simultaneous access to all vertebrae in which the surgical procedure is being performed. In some cases, however, additional enlargement or repositioning of the distal portion of the access device 20 may be helpful in providing sufficient access to the outer vertebrae, e.g., the L4 and S1 vertebrae. In the illustrated embodiment, the expander apparatus 200 may be repeatedly inserted into the access device 20 and expanded in order to further open or to position the skirt portion 24. In one procedure, additional fasteners are inserted in the L4 and S1 vertebrae in a similar fashion as the fastener 600 inserted into the L5 vertebra as described above. (When discussed individually or collectively, a fastener and/or its individual components will be referred to by the reference number, e.g., fastener 600, housing 604, and all fasteners 600. However, when several fasteners and/or their components are discussed in relation to one another, an alphabetic subscript will be used, e.g., fastener 600a is moved towards fastener 600b.)

In one application, after the fasteners 600 are advanced into the vertebrae, the housing portions 604 of the fasteners 600 are substantially aligned such that their upright portions 630 and 631 face upward, and the notches 632 are substantially aligned to receive the elongated member 650 therein. The frictional mounting of the housing 604 to the screw member 602, described above, allows the housing 604 to be temporarily positioned until a subsequent tightening step is performed, described below.

Positioning of the housing portions 604 may be performed by the use of an elongated surgical instrument capable of contacting and moving the housing portion to the desired orientation. One such instrument for positioning the housings 604 is a grasped apparatus 700, illustrated in FIG. 30. The grasper apparatus 700 includes a proximal handle portion 702, an elongated body portion 704, and distal nose portion 706. The distal nose portion 706 includes a pair of grasping jaws 708a and 708b, which are pivotable about pin 710 by actuation of the proximal handle portion 702. The grasping jaws 708a and 708b are illustrated in the closed position in FIG. 30. Pivoting the movable handle 714 towards stationary handle 712 causes longitudinal movement of actuator 716, which in turn pivots the jaw 708b towards an open position (illustrated in dashed line). The biasing members 718 and 720 are provided to return the handles 712 and 714 to the open position and bias the jaws 708a and 708b to the closed position.

In one application, the elongated member 650 is inserted into the access device 20. In one application, the elongated member 650 is manufactured from a biocompatible material and is sufficiently strong to maintain the position of the vertebrae, or other body structures, coupled by the elongate member 650 with little or no relative motion therebetween. In one embodiment, the elongated members 650 are manufactured from Titanium 6/4 or titanium alloy. The elongated member 650 also may be manufactured from stainless steel or any other suitable material. The transverse shape, width (e.g., radii), and lengths of the elongated members 650 are selected by the physician to provide the best fit for the positioning of the screw heads. Such selection may be performed by placing the elongated member 650 on the skin of the patient overlying the location of the fasteners and viewed fluoroscopically. For example, a 70 mm preformed rod having a 3.5″ bend radius may be selected for the spinal fixation.

In one application, the elongated member 650 is fixed to each of the fasteners 600, and more particularly, to the housings 604 of each fastener 600. The grasper apparatus 700, described above, is also particularly useful for inserting the elongated member 650 into the access device 20 and positioning it with respect to each housing 604. As illustrated in FIG. 30, the jaws 708a and 708b of the grasper apparatus 700 each has shaped (e.g., curved) contact portions 722a and 722b for contacting and holding the outer surface of the elongated member 650.

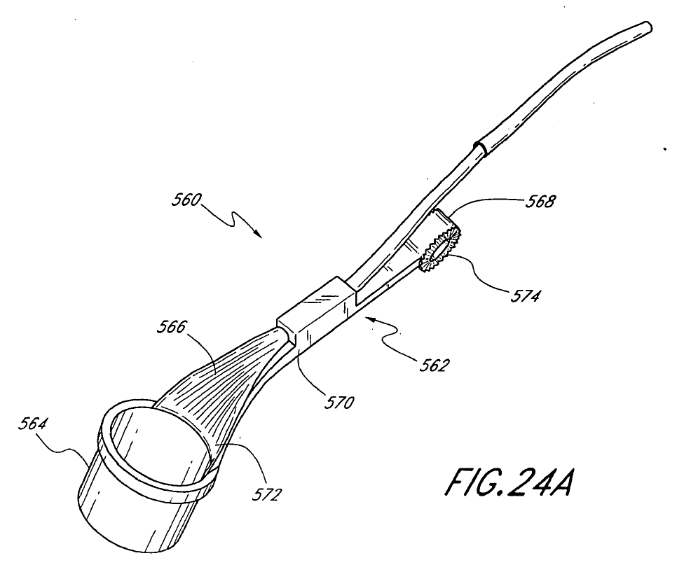

As illustrated in FIG. 31, the grasper apparatus 700 may be used to insert the elongated member 650 into the operative space 90 defined at least partially by the skirt portion 24 of the access device 20. In some embodiments, the cut-out portions 56 and 58 provided in the skirt portion 24 assist in the process of installing the elongated member 650 with respect to the housings 604. The cut-out portions 56 and 58 allow an end portion 652 of the elongated member 650 to extend beyond the operative space without raising or repositioning the skirt portion 24. The elongated member 650 is positioned within the recesses in each housing 604 defined by grooves 632 disposed between upright members 630 and 631. The elongated member 650 is positioned in an orientation substantially transverse to the longitudinal axis of each housing 604.

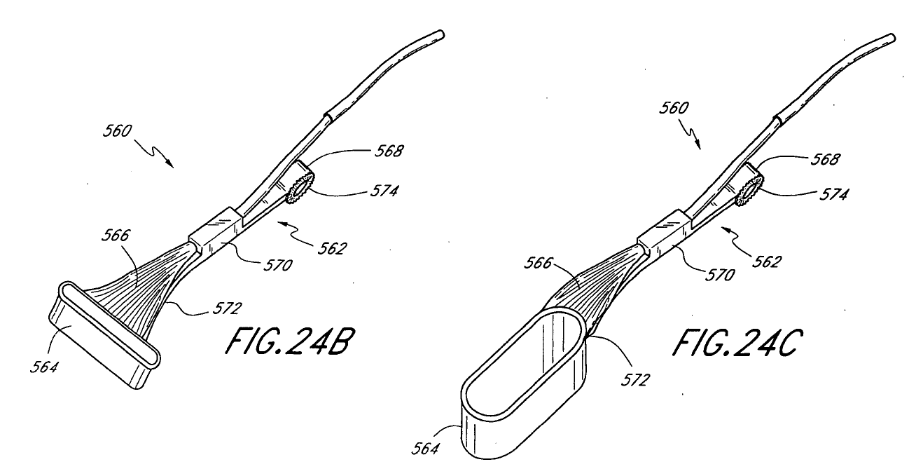

Further positioning of the elongated member 650 may be performed by guide apparatus 800, illustrated in FIG. 32. Guide apparatus 800 is useful in cooperation with an endoscopic screwdriver, such as endoscopic screwdriver 660 (illustrated in FIG. 28), in order to position the elongated member 650, and to introduce and tighten the cap screw 610, described above and illustrated in FIG. 27. Tightening of the cap screw 610 with respect to the housing 604 fixes the orientation of the housing 604 with respect to the screw portion 602 and fixes the position of the elongated member 650 with respect to the housings 604.

In the illustrated embodiment, the guide apparatus 800 has a proximal handle portion 802, an elongated body portion 804, and a distal tool portion 806. The elongated body portion 804 defines a central bore 808 (illustrated in dashed line) along its longitudinal axis 810. The central bore 808 is sized and configured to receive the endoscopic screwdriver 660 and cap screw 610 therethrough. In the exemplary embodiment, the diameter of the central bore 808 of the elongated body portion 804 is about 0.384-0.388 inches in diameter, and the external diameter of the endoscopic screwdriver 660 (FIG. 28) is about 0.25 inches. The proximal handle portion 802 extends transverse to the longitudinal axis 810, which allows the physician to adjust the guide apparatus 800 without interfering with the operation of the screwdriver 660.

The distal portion 806 of the apparatus includes several shaped cut out portions 814 which assist in positioning the elongated member 650. As illustrated in FIG. 33, the cut out portions 814 are sized and configured to engage the surface of elongated member 650 and move the elongated member 650 from an initial location (illustrated in dashed line) to a desired location. In the illustrated embodiment, the cut out portions 814 are semicircular, to match the round elongated member 650. However, other shaped cut out portions may be provided to match other shaped elongated members.

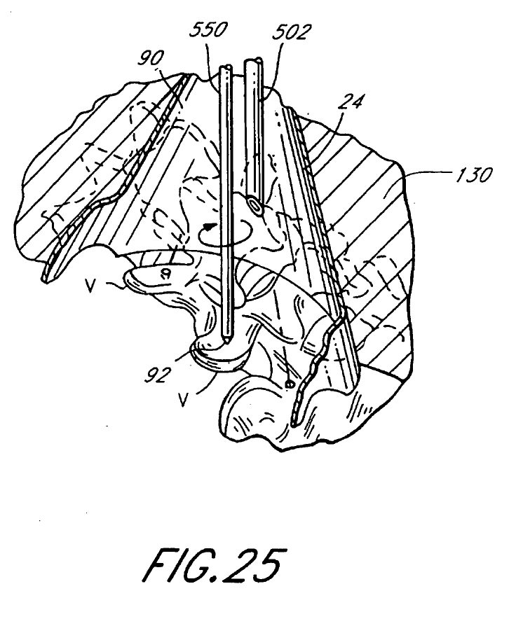

As illustrated in FIG. 34, the guide apparatus 800 is used in cooperation with the endoscopic screwdriver 660 to attach the cap screw 610. The distal end of the body portion 804 includes a pair of elongated openings 816. The openings 816 provide a window to enable the physician to endoscopically view the cap screw 610 retained at the distal tip 666 of the endoscopic screw driver 660. Fewer or more than two openings can be provided and the openings 816 need not be elongated.

The guide apparatus 800 and the endoscopic screwdriver 660 cooperate as follows in one application. The guide apparatus 800 is configured to be positioned in a surrounding configuration with the screwdriver 600. In the illustrated embodiment, the body portion 804 is configured for coaxial placement about the screwdriver 660 in order to distribute the contact force of the guide apparatus 800 on the elongated member 650. The distal portion 806 of the guide apparatus 800 may bear down on the elongated member 650 to seat the elongated member 650 in the notches 632 in the housing 604. The “distributed” force of the guide apparatus 800 may contact the elongated member 650 on at least one or more locations. In addition, the diameter of central bore 808 is selected to be marginally larger than the exterior diameter of cap screw 610, such that the cap screw 610 may freely slide down the central bore 808, while maintaining the orientation shown in FIG. 34, This configuration allows the physician to have effective control of the placement of the cap screw 610 into the housing 604. The cap screw 610 is releasably attached to the encloscopic screwdriver 660 by means of spring member 672 engaged to the interior wall of hexagonal recess 611 as it is inserted within the bore 808 of the body portion 804 of guide apparatus 800. The cap screw 610 is attached to the housing 604 by engaging the threads 615 of the cap screw 610 with the threads 634 of the housing.

As illustrated in FIG. 35, tightening of the cap screw 610 fixes the assembly of the housing 604 with respect to the elongated member 650. In particular, the distal surface of the cap screw 610 provides a distal force against the elongated member 650, which in turn drives the spacer member 606 against the joint portion 614 of the screw portion 602, which is fixed with respect to the housing 604.

If locations of the vertebrae are considered acceptable by the physician, then the fixation procedure is substantially complete once the cap screws 610 have been attached to the respective housings 604, and tightened to provide a fixed structure as between the elongated member 650 and the various fasteners 600. However, if compression or distraction of the vertebrae with respect to one another is required additional apparatus would be used to shift the vertebrae prior to final tightening all of the cap screws 610.

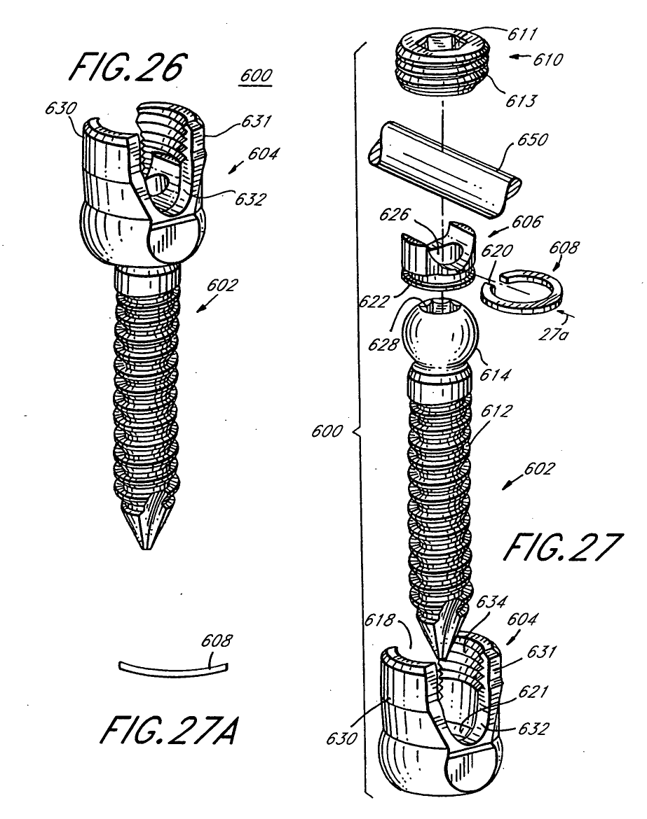

In the illustrated embodiment, this step is performed with a surgical instrument, such as a compressor-distractor instrument 900, illustrated in FIG. 36, which is useful to relatively position bone structures in the cephcaudal direction and to fix their position with respect to one another. Thus, the compressor-distractor instrument 900 has the capability to engage two fasteners 600 and to space them apart while simultaneously tightening one of the fasteners to fix the spacing between the two vertebrae, or other bone structures. Moreover, the compressor-distractor instrument 900 may also be used to move two fasteners 600, and the vertebrae attached thereto into closer approximation and fix the spacing therebetween.

The distal tool portion 902 of one embodiment of the compressor-distractor instrument 900 is illustrated in FIG. 36. The distal tool portion 902 includes a driver portion 904 and a spacing member 906. The driver portion 904 has a distal end portion 908 with a plurality of wrenching flats configured to engage the recess 611 in the proximal face of the cap screw 610, and to apply torque to the cap screw. The driver portion 904 is rotatable about the longitudinal axis (indicated by arrow M) to rotate the cap screw 610 relative to the fastener 600. Accordingly, the driver portion 904 can be rotated to loosen the cap screw 610 on the fastener 600 and permit movement of the elongated member 650 connected with the vertebra relative to the fastener 600 connected with the vertebra. The cap screw 610 can also be rotated in order to tighten the cap screw 610 and clamp the elongated member 650 to the fastener 600.

The distal tool portion 902 may also include a spacing member, such as spacing member 906, which engages an adjacent fastener 600b while driver member 904 is engaged with the housing 604a to move the fastener 600b with respect to the fastener 600a. In the exemplary embodiment, spacing member 906 comprises a jaw portion that is pivotably mounted to move between a first position adjacent the driver portion and a second position spaced from the driver portion, as shown in FIG. 36. The distal tip 910 of the spacing member 906 is movable relative to the driver portion 904 in a direction extending transverse to the longitudinal axis. (Further details and features related to compressor-distractor apparatuses are described in U.S. application Ser. No. 10/178,875, filed Jun. 24, 2002, entitled “Surgical Instrument for Moving Vertebrae,” published as U.S. Patent Application Publication No. 2003/0236529A1 on Dec. 25, 2003, which is incorporated by reference in its entirety herein. Additionally, further details related to instrumentation for moving a vertebra are described in U.S. Pat. No. 6,648,888, issued Nov. 18, 2003; PCT Application No. PCT/US02/28106, filed Sep. 5, 2002, entitled SURGICAL INSTRUMENT FOR MOVING VERTEBRAE; PCT Application No. PCT/US03/27879, filed Sep. 5, 2003, entitled SURGICAL INSTRUMENT FOR MOVING A VERTEBRAE; and PCT Application No. PCT/US03/04361, filed Feb. 13, 2003, entitled APPARATUS FOR CONNECTING A LONGITUDINAL MEMBER TO A BONE PORTION, which are hereby incorporated by reference in their entireties herein.)

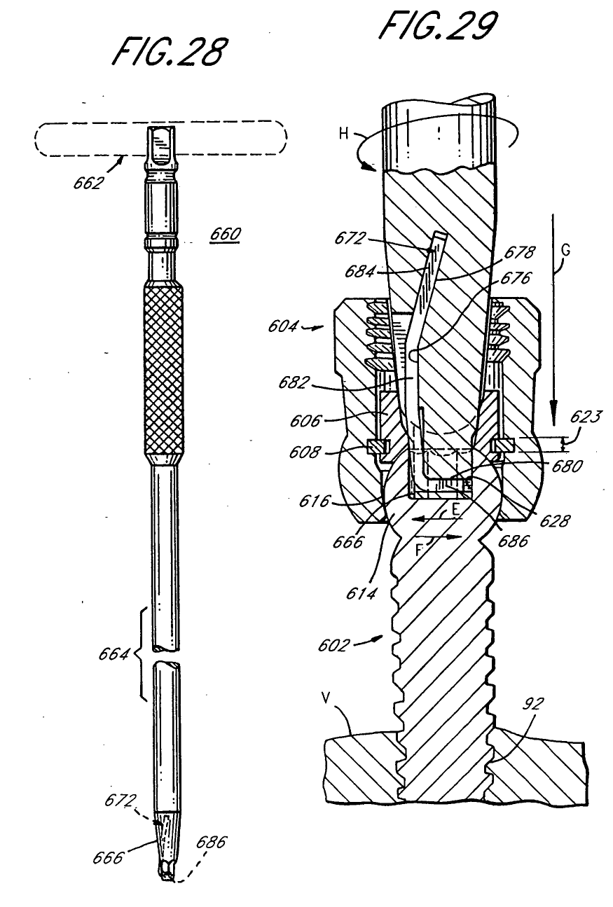

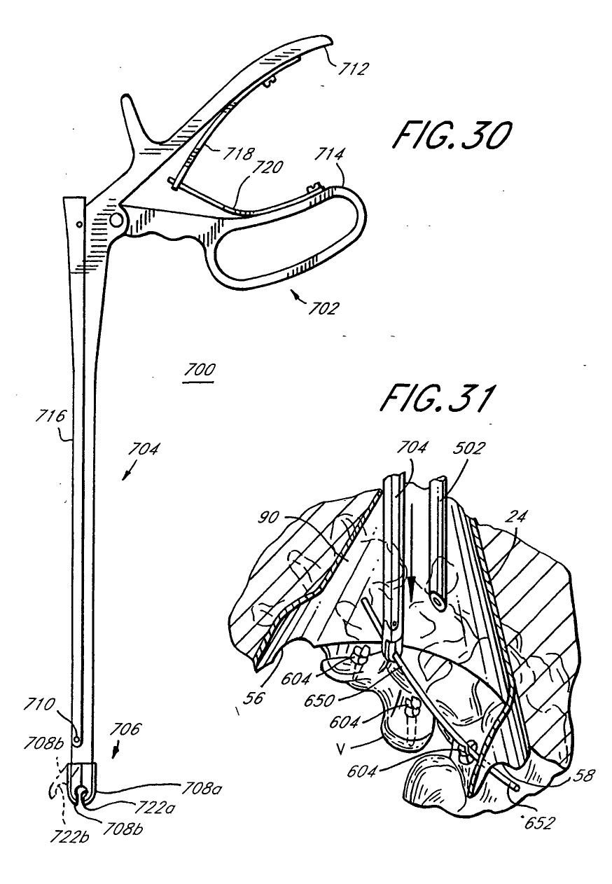

As illustrated in FIG. 36, the spacer member 906 can be opened with respect to the driver portion 904 to space the vertebrae farther apart (as indicated by arrow N). The distal portion 910 of the spacer member 906 engages the housing 604b of fastener 600b and moves fastener 600b further apart from fastener 600a to distract the vertebrae. Where the vertebrae are to be moved closer together, e.g. compressed, the spacer member 906 is closed with respect to the driver portion 904 (arrow P), as illustrated in FIG. 37. The distal portion 910 of the spacer member 906 engages the housing 604b of the fastener 600b and moves the fastener 600b towards the fastener 600a. When the spacing of the vertebrae is acceptable to the physician, the cap screw 610a is tightened by the driver member 904, thereby fixing the relationship of the housing 604a with respect to the elongated member 650, and thereby fixing the position of the vertebrae, or other bone structures, with respect to one another. In one application, once the elongated member 650 is fixed with respect to the fasteners 600, the fixation portion of the procedure is substantially complete.

2. Fusion Systems and Devices

Although fixation may provide sufficient stabilization, in some cases it is also desirable to provide additional stabilization. For example, where one or more discs has degraded to the point that it needs to be replaced, it may be desirable to position an implant, e.g., a fusion device, a prosthetic disc, a disc nucleus, etc., in the intervertebral space formerly occupied by the disc.

In one application, a fusion device is inserted between adjacent vertebrae V. Portions of the fusion procedure can be performed before, during, or after portions of the fixation procedure. FIGS. 38-42 illustrate one embodiment of a fusion device, referred to herein as a spinal implant 2010, that is inserted between adjacent vertebrae. The spinal implant 2010 preferably is placed between adjacent vertebrae to provide sufficient support to allow fusion of the adjacent vertebrae, as shown in FIGS. 48-49. The spinal implants 2010 are preferably made from an allograft material, though other materials could also be used, including autograft, xenograft, or some non-biologic biocompatible material, such as titanium or stainless steel. Also, where non-biologic materials are used, the implant 2010 may be configured as a cage or other suitable configuration.

The spinal implant 2010 (FIGS. 38-42) has a first end 2020 for insertion between adjacent vertebrae V. The first end 2020 has a tapered surface 2022 to facilitate insertion of the implant between adjacent vertebrae V. The surface 2022 defines an angle X of approximately 45° as shown in FIG. 41.

The spinal implant 2010 (FIGS. 38-39) has a second end 2030 that is engageable with a tool 2032 (FIG. 51) for inserting the implant between the adjacent vertebrae V. The tool 2032 has a pair of projections 2034, one of which is shown in FIG. 51, that extend into recesses 2036 and 2038 in the end 2030 of the implant 2010. The recesses 2036 and 2038 (FIGS. 38-39) extend from the second end 2030 toward the first end 2020. The recess 2036 (FIG. 41) is defined by an upper surface 2040 and a lower surface 2042 extending generally parallel to the upper surface 2040. The recess 2038 (FIG. 39) has a lower surface 2046 and an upper surface 2048. The upper surface 2048 extends generally parallel to the lower surface 2046.

The recesses 2036 and 2038 define a gripping portion 2052. The projections 2034 on the tool 2032 extend into the recesses 2036 and 2038 and grip the gripping portion 2052. The projections 2034 engage the upper and lower surfaces 2040 and 2042 of the recess 2036 and the upper and lower surfaces 2046 and 2048 of the recess 2038. Accordingly, the tool 2032 can grip the implant 2010 for inserting the implant between the adjacent vertebrae V.

As viewed in FIGS. 38-41, the implant 2010 has an upper surface 2060 for engaging the upper vertebra V. The implant 2010 has a lower surface 2062, as viewed in FIGS. 38-41, for engaging the lower vertebra V. The upper and lower surfaces 2060 and 2062 extend from the first end 2020 to the second end 2030 of the implant 2010 and parallel to the upper and lower surfaces 2040, 2042, 2046, and 2048 of the recesses 2036 and 2038. The upper surface 2060 has teeth 2064 for engaging the upper vertebra V. The lower surface 2062 has teeth 2066 for engaging the lower vertebra V. Although FIGS. 38-39 show four teeth 2064 and four teeth 2066, it is contemplated that any number of teeth could be used.

A first side surface 2070 and a second side surface 2072 extend between the upper and lower surfaces 2060 and 2062. The first side surface 2070 extends along a first arc from the first end 2022 of the implant 2010 to the second end 2030. The second side surface 2072 extends along a second arc from the first end 2022 to the second end 2030. The first and second side surfaces 2070 and 2072 are concentric and define portions of concentric circles. The teeth 2064 and 2066 extend parallel to each other and extend between the side surfaces 2070 and 2072 and along secant lines of the concentric circles defined by the side surfaces.

The implant 2010 preferably is formed by harvesting allograft material from a femur, as known in the art. The femur is axially cut to form cylindrical pieces of allograft material. The cylindrical pieces are then cut in half to form semi-cylindrical pieces of allograft material. The semi-cylindrical pieces of allograft material are machined into the spinal implants 2010.

A pair of spinal implants 2010 may be placed bilaterally between the adjacent vertebrae V. The access device 20 is positioned in the patient's body adjacent the vertebrae V. The skirt portion 24 of the access device 20 preferably is in a radially expanded condition to provide a working space adjacent the vertebrae V as described above. Disc material between the vertebrae V can be removed using instruments such as kerrisons, rongeurs, or curettes. A microdebrider may also be utilized to remove the disc material. An osteotome, curettes, and scrapers can be used to prepare end plates of the vertebrae V for fusion. Preferably, an annulus of the disc is left between the vertebrae V.

Distracters can be used to sequentially distract the disc space until the desired distance between the vertebrae V is achieved. The fusion device or implant 2010 is placed between the vertebrae V using the tool 2032. The first end 2020 of the implant 2010 is inserted first between the vertebrae V. The implant 2010 is pushed between the vertebrae V until the end 2030 of the implant is between the vertebrae. A second spinal implant 2010 is inserted on the ipsilateral side using the same procedure.

A shield apparatus 3100 with an elongated portion 3102 may be used to facilitate insertion of the implants 2010 between the vertebrae V. A distal portion 3110 of the apparatus 3100 may be placed in an annulotomy. The implant 2010 is inserted with the side surface 2170 facing the elongated portion 3102 so that the apparatus 3100 can act as a “shoe horn” to facilitate or guide insertion of the implants 2010 between the vertebrae.

The implants 2010 may be inserted between the vertebrae V with the first ends 2020 located adjacent each other and the second ends 2030 spaced apart from each other, as shown in FIG. 48. The implants 2010 may also be inserted between the vertebrae V with the first ends 2020 of the implants 2010 spaced apart approximately the same distance that the second ends 2030 are spaced apart. It is contemplated that the implants 2010 may be inserted in any desired position between the vertebrae V. It is also contemplated that in some embodiments only one implant 2010 may be inserted between the vertebrae V. Furthermore, it is contemplated that the implants 2010 may be inserted between vertebrae using an open procedure.

Another embodiment of a fusion device or spinal implant 2110 is illustrated in FIGS. 43-47. The spinal implant 2110 is substantially similar to the embodiment disclosed in FIGS. 38-42. The implant 2110 is placed between the adjacent vertebrae V to provide sufficient support to allow fusion of the adjacent vertebrae, as shown in FIG. 50. The spinal implant 2110 is preferably made from an allograft material, though the materials described above in connection with the spinal implant 2010 may also be used. Also, as with the implant 2010, the implant 2110 may be formed as a cage or other suitable configuration.

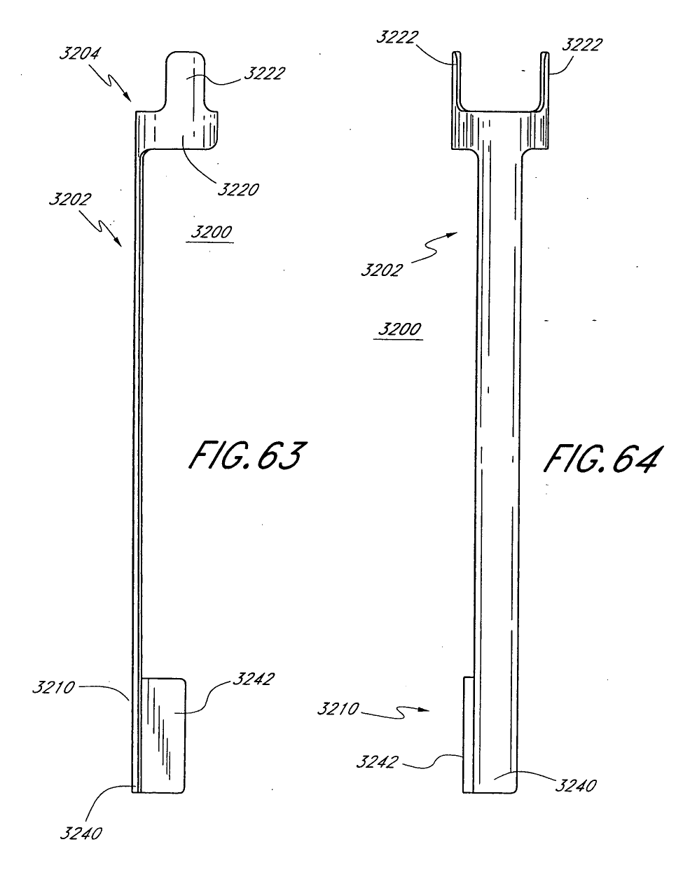

The spinal implant 2110 (FIGS. 43-47) has a first end 2120 for insertion between the adjacent vertebrae V. The first end 2120 has a tapered surface 2122 to facilitate insertion of the implant between the adjacent vertebrae V. The surface 2122 defines an angle Y of approximately 45° as shown in FIG. 65.

The spinal implant 2110 (FIGS. 43-44) has a second end 2130 that is engageable with the projections 2034 on the tool 2032 for inserting the implant between the adjacent vertebrae V. The projections 2034 extend into recesses 2136 and 2138 in the end 2130 of the implant 2110. The recesses 2136 and 2138 extend from the second end 2130 toward the first end 2120. The recess 2136 (FIGS. 43 and 46) is defined by an upper surface 2140 and a lower surface 2142 extending generally parallel to the upper surface 2140. The recess 2138 (FIGS. 44) has a lower surface 2146 and an upper surface 2148 extending generally parallel to the lower surface 2146.

The recesses 2136 and 2138 define a gripping portion 2152. The projections 2034 on the tool 2032 extend into the recesses 2136 and 2138 and grip the gripping portion 2152. The projections 2034 engage the upper and lower surfaces 2140 and 2142 of the recess 2136 and the upper and lower surfaces 2146 and 2148 of the recess 2138. Accordingly, the tool 2032 can grip the implant 2110 for inserting the implant between the adjacent vertebrae V.

As viewed in FIGS. 43-46, the implant 2110 has an upper surface 2160 for engaging the upper vertebra V. The implant 2110 has a lower surface 2162, as viewed in FIGS. 43-46, for engaging the lower vertebra V. The upper and lower surfaces 2160 and 2162 extend from the first end 2120 to the second end 2130 of the implant 2110 and parallel to the upper and lower surfaces 2140, 2142, 2146, and 2148 of the recesses 2136 and 2138. The upper surface 2160 has teeth 2164 for engaging the upper vertebra V. The lower surface 2162 has teeth 2166 for engaging the lower vertebra V. Although FIG. 44 shows four teeth 2164 and four teeth 2166, it is contemplated that any number of teeth could be used.

A first side surface 2170 and a second side surface 2172 extend between the upper and lower surfaces 2160 and 2162. The first side surface 2170 extends along a first arc from the first end 2122 of the implant 2110 to the second end 2130. The second side surface 2172 extends along a second arc from the first end 2120 to the second end 2130. The first and second side surfaces 2170 and 2172 are concentric and define portions of concentric circles. The teeth 2164 and 2166 extend parallel to each other and between the side surfaces 2170 and 2172 along secant lines of the concentric circles defined by the side surfaces.

The implant 2110 preferably is formed by harvesting allograft material from a femur, as is known in the art. The femur is axially cut to form cylindrical pieces of allograft material. The cylindrical pieces are then cut in half to form semi-cylindrical pieces of allograft material. The semi-cylindrical pieces of allograft material are machined into the spinal implants 2110.

A spinal implant 2110 is placed unilaterally between the adjacent vertebrae V. The access device 20 is positioned in the patient's body adjacent the vertebrae V. The skirt portion 24 of the access device 20 preferably is in a radially expanded condition to provide a working space adjacent the vertebrae V as described above. Disc material between the vertebrae V can be removed using instruments such as kerrisons, rongeurs, or curettes. A microdebrider may also be utilized to remove the disc material. An osteotome, curettes, and scrapers can be used to prepare end plates of the vertebrae V for fusion. Preferably, an annulus of the disc is left between the vertebrae V. 102271 Distracters are used to sequentially distract the disc space until the desired distance between the vertebrae V is achieved. The implant 2110 is placed between the vertebrae V using the tool 2032. It is contemplated that the apparatus 3100 could be used also. The first end 2120 of the implant 2110 is inserted first between the vertebrae V. The implant 2110 is pushed between the vertebrae V until the end 2130 of the implant is between the vertebrae. It is contemplated that the implant 2110 may be inserted in any desired position between the vertebrae V. It is also contemplated that in some embodiments more than one implant 2110 may be inserted between the vertebrae.

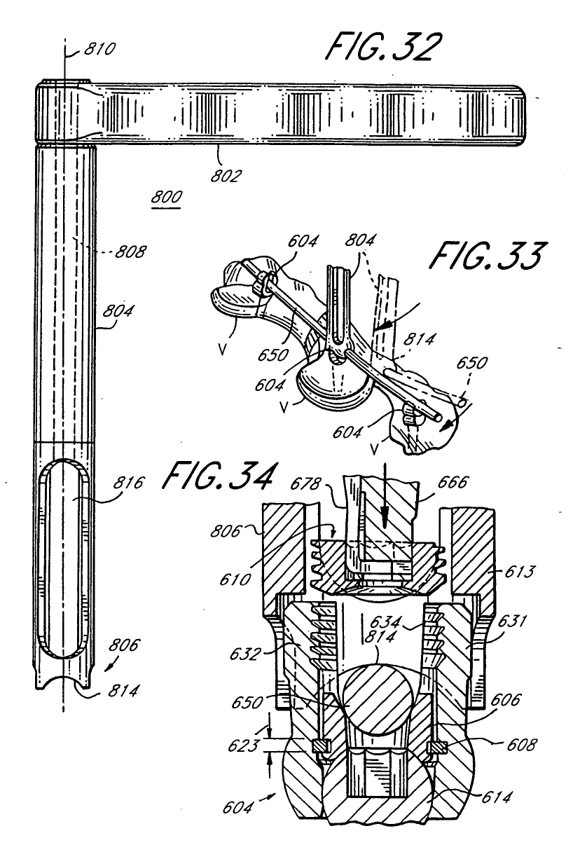

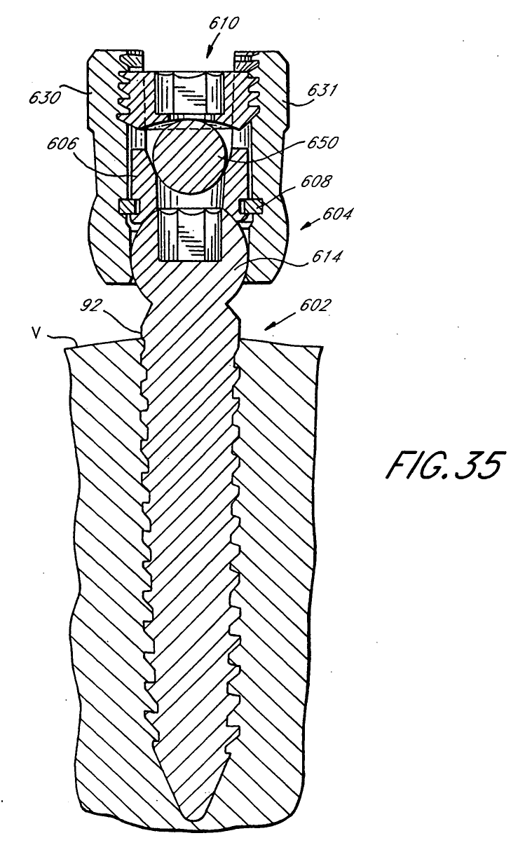

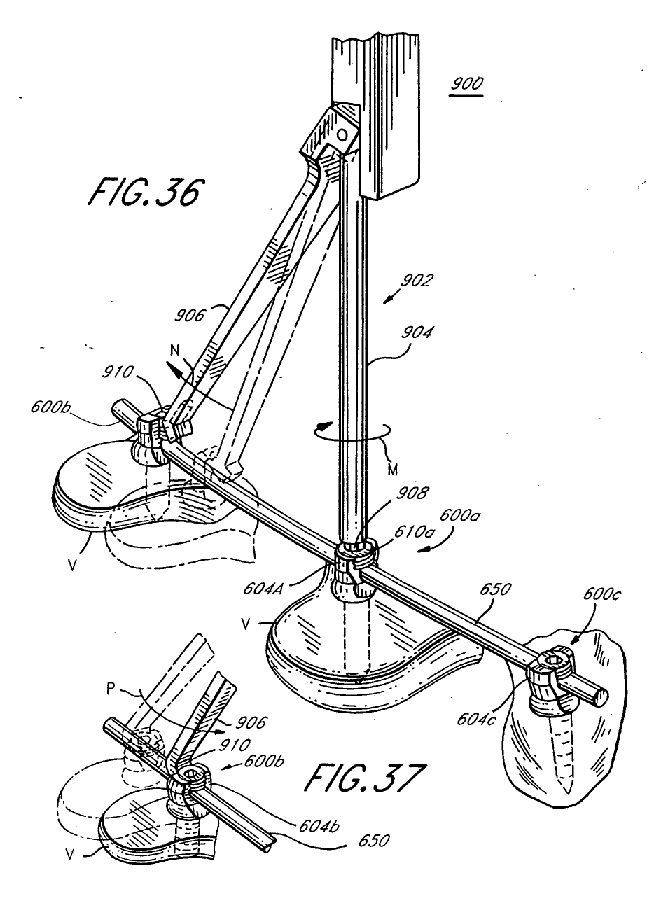

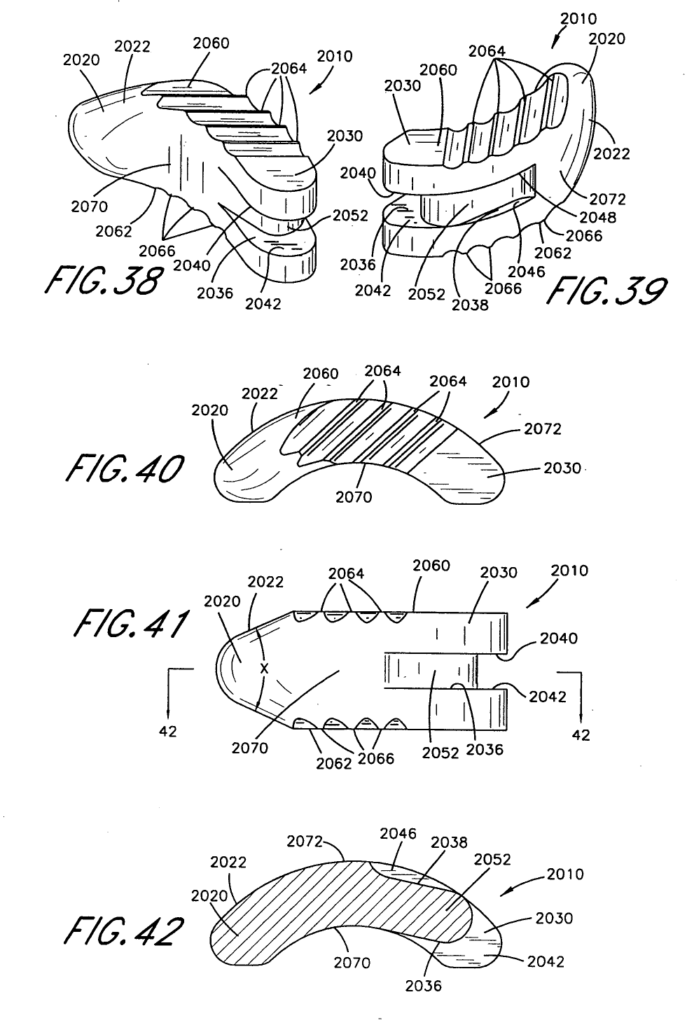

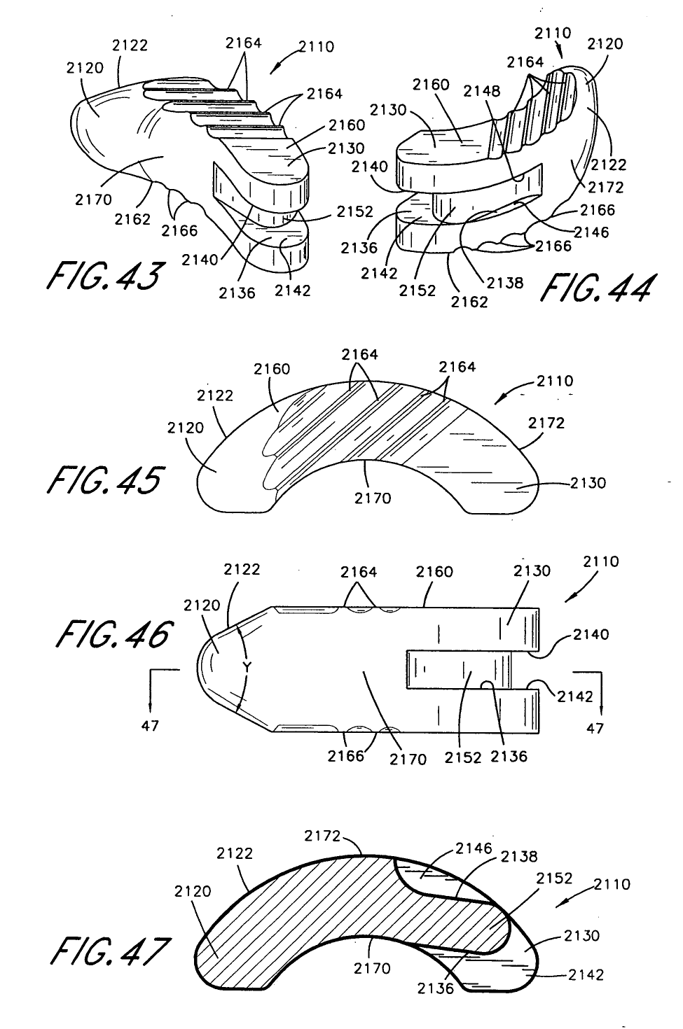

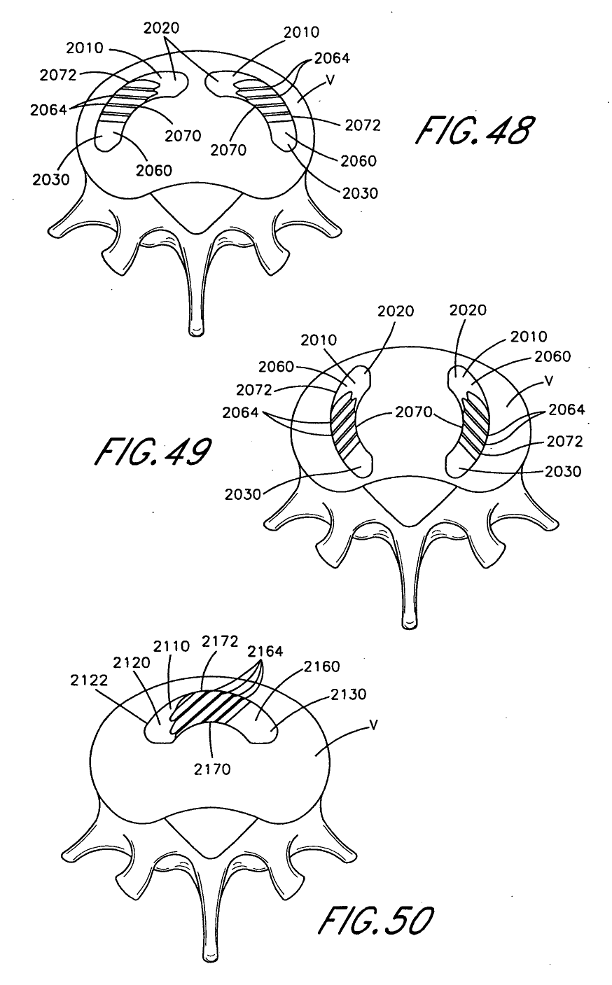

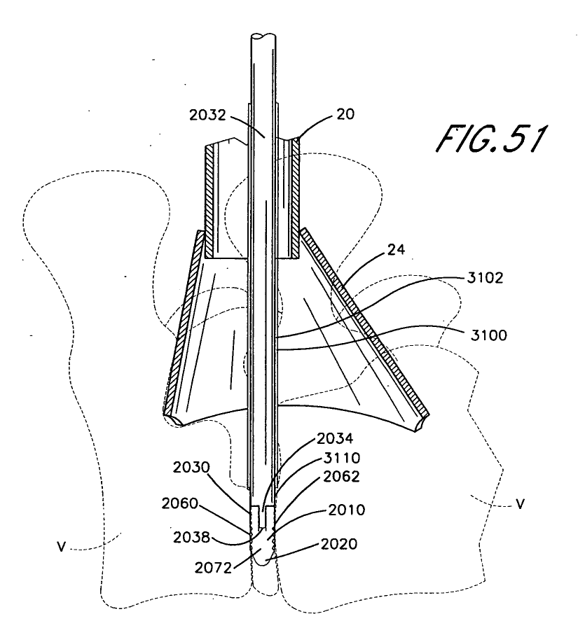

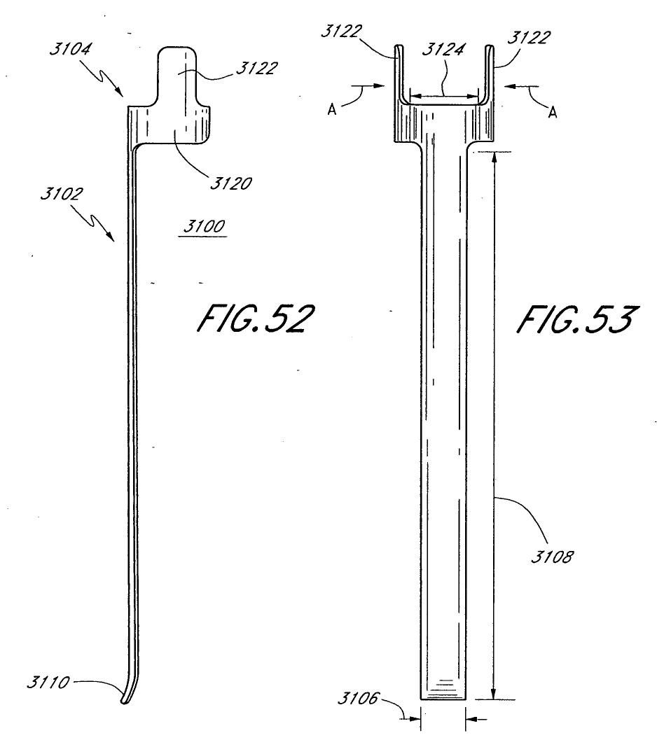

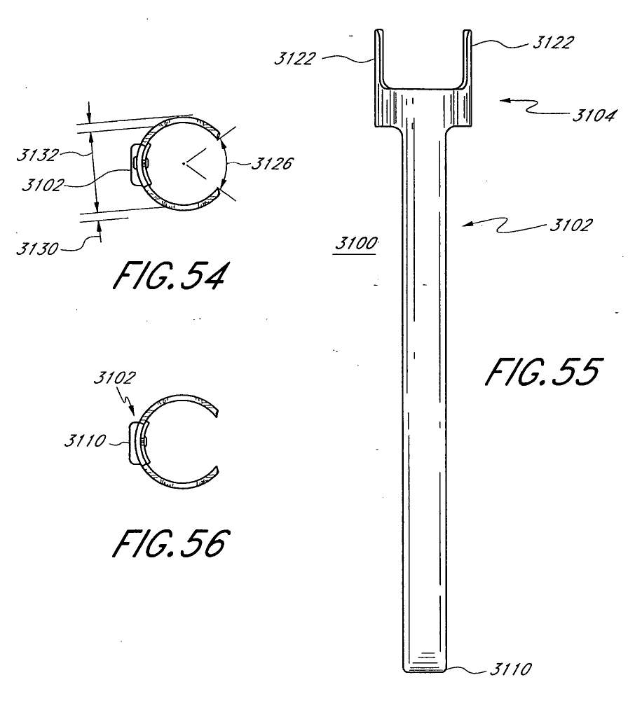

The apparatus or shield 3100 for use in placing the fusion devices or spinal implants between the vertebrae is illustrated in FIGS. 52-56. The apparatus 3100 preferably includes an elongated body portion 3102, which protects the nerve root or dura, and a mounting portion 3104, which allows for the surgeon to releasably mount the apparatus 3100 to the access device 20. Consequently, the surgeon is able to perform the surgical procedures without requiring the surgeon or an assistant to continue to support the apparatus 3100 throughout the procedure, and without reducing the field of view.

The apparatus 3100 may be manufactured from a biocompatible material such as, for example, stainless steel. In the illustrated embodiment, apparatus 3100 is manufactured from stainless steel having a thickness of about 0.02 inches to about 0.036 inches. The elongated body portion 3102 has dimensions that correspond to the depth in the body in which the procedure is being performed, and to the size of the body structure that is to be shielded by elongated body portion 3102. In the exemplary embodiment, the elongated body portion 3102 has a width 3106 of about 0.346 inches and a length of about 5.06 inches (FIG. 53), although other dimensions would be appropriate for spinal surgical procedures performed at different locations, or for surgical procedures involving different body structures. The distal tip portion 3110 of the apparatus 3100 may have a slightly curved “bell mouth” configuration which allows for atraumatic contact with a body structure, such as a nerve. It is contemplated that the elongated body portion may have any desired shape.

The mounting portion 3104 preferably allows the apparatus 3100 to be secured to a support structure in any number of ways. In the exemplary embodiment, mounting portion 3104 may include a ring portion. With reference to FIGS. 52-56, ring portion 3120 has a substantially ring-shaped configuration with an opening 3124, which defines an angle 3126 of about 90 degrees of the total circumference of the ring portion 3120. As will be described in greater detail below, the angle 3126 is a nominal value, because the ring portion 3104 is resilient, which permits the opening 3124 to change size during the mounting process.

In the illustrated embodiment, the mounting portion 3104 has a substantially cylindrical configuration in order to be mounted within the interior lumen of the access device 20, as will be described below. The ring portion 3104 has an exterior dimension 3130 of about 0.79 inches, and an interior dimension 3132 of about 0.76 inches. It is understood that the dimensions of the ring portion 3104 can be different, such as, for example, where the access device 20 has a different interior dimension. Moreover, the cylindrical shape of the ring portion 3104 can change, such as, for example, where the apparatus 3100 is used with a support member having a differently shaped internal lumen.

Finger grip portions 3122 preferably extend from the mounting portion 3104 and allow the surgeon to apply an inwardly directed force (as indicated by arrows A) to the ring portion 3120. The resilient characteristics of the ring portion 3120 allow the material to deflect thereby reducing the exterior dimension 3130 and reducing the spacing 3124. Releasing the finger grip portions 3122 allows the ring portion to move towards its undeflected condition, thereby engaging the interior wall of the access device 20.

The elongated body portion 3102 and the mounting portion 3104 may be manufactured from a single component, such as a sheet of stainless steel, and the mounting portion 3104 may be subsequently formed into a substantially cylindrical shape. In another embodiment, the mounting portion 3104 may be manufactured as a separate component and coupled to the elongated body portion, by techniques such as, for example, welding and/or securement by fasteners, such as rivets.

The access device 20 serves as a stable mounting structure for apparatus 3100. In particular, mounting portion 3104 is releasably mounted to the interior wall of proximal wall portion 22 of access device 20. Elongated body portion 3102 extends distally into the operative site to protect the desired body structure, such as the nerve, as will be described below.

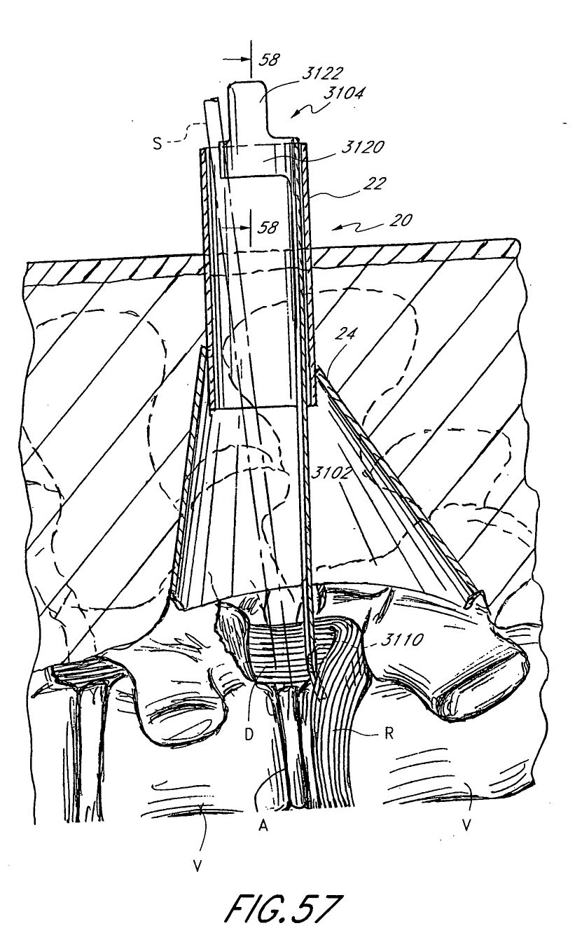

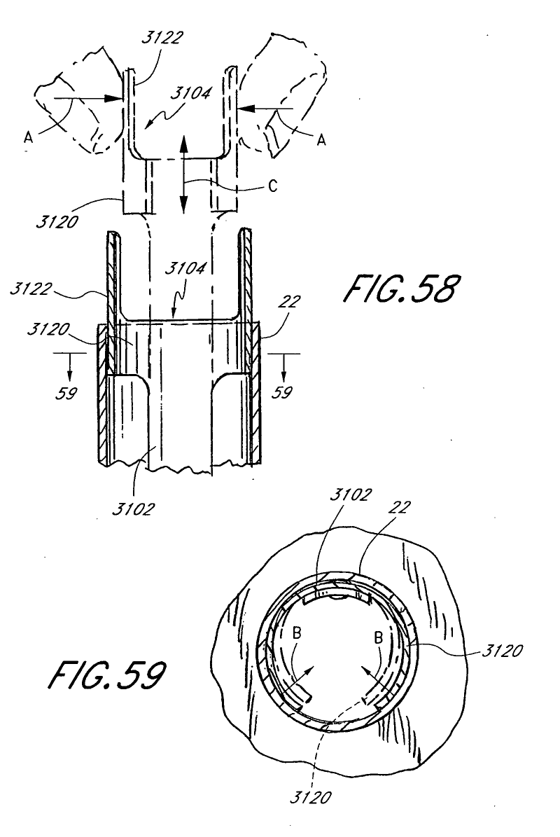

To install the apparatus 3100 within the interior passage of the proximal wall portion 22, the surgeon may apply an inwardly directed force on the ring portion 3120, thereby causing the ring portion to resiliently deform, as illustrated by dashed line and arrows B in FIG. 59. The surgeon subsequently inserts the apparatus 3100 into the interior lumen of the proximal wall portion 22 (as indicated by arrow C) to the position of ring portion 3104 illustrated in solid line in FIG. 58. When the surgeon releases the finger grip portions 3122, the ring portion 3120 resiliently moves towards its undeflected configuration, thereby engaging the interior lumen of the proximal wall portion 22. Advantages of some embodiments include that the mounting portion 3104 is easily removed and/or moved with respect to the access device 20 without disturbing the position of the access device 20 or any other instrumentation.

As illustrated in FIG. 57, the configuration of the mounting portion 3104 and the elongated body portion 3102 allow the elongated body portion to occupy a small space along the periphery of the proximal wall portion 3122. This allows the apparatus to protect the desired body structure without blocking access for the insertion of other surgical instrumentation, and without blocking visibility by the surgeon during the procedure.

The mounting portion 3104 is one exemplary configuration for mounting the apparatus 3100 to the support structure. It is contemplated that the apparatus 3100 may be mounted within the access device 20 in any suitable manner.

When in position, the distal end portion 3110 covers the exiting nerve root R, while exposing the disc annulus A (See FIG. 57). As discussed above, the debridement and decortication of tissue covering the vertebrae, as well as a facetectomy and/or laminectomy if indicated, are preferably performed prior to the insertion of apparatus 3100 into the surgical space. Accordingly, in some embodiments, there is no need to displace or retract tissue, and apparatus 3100 merely covers the nerve root and does not substantially, displace the nerve root or any other body tissue. It is understood that the term “cover” as used herein refers to apparatus 3100 being adjacent to the body structure, or in contact with the body structure without applying significant tension or displacement force to the body structure.

Additional surgical instrumentation S may be inserted into the access device to perform procedures on the surrounding tissue. For example, an annulotomy may be performed using a long handled knife and kerrisons. A discectomy may be completed by using curettes and rongeurs. Removal of osteophytes which may have accumulated between the vertebrae may be performed using osteotomes and chisels.

As illustrated in FIG. 60, the elongated body portion 3102 preferably is rotated to protect the spinal cord, or dura D, during the above procedures. The surgeon may change the position of the apparatus 3100 by approximating the finger grips 3122 to release the ring portion from engagement with the inner wall of the proximal wall portion 20, and then re-position the apparatus 3100 without disturbing the access device 20 (as shown in FIG. 58).

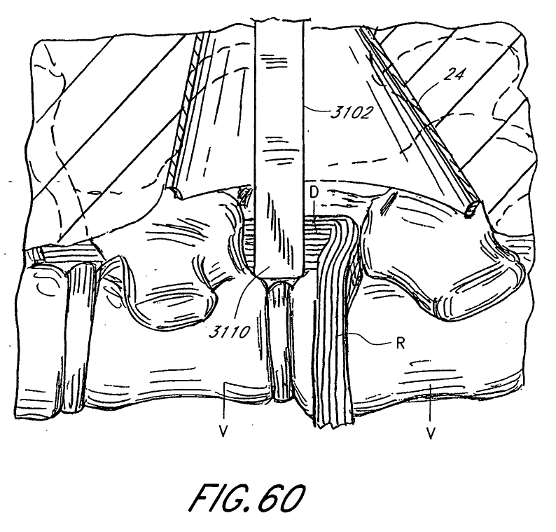

During certain surgical procedures, it may be useful to introduce crushed bone fragments or the fusion devices 2010 or 2110 to promote bone fusion. As illustrated ill FIGS. 61-62, apparatus 3100 is useful to direct the implants into the space I between adjacent vertebrae V. As shown in the figures, the distal portion 3110 of the elongated body portion 3102 is partially inserted into the space I. The distal end portion 3110, is positioned between adjacent vertebrae V, and creates a partially enclosed space for receiving the implants or other material therein.

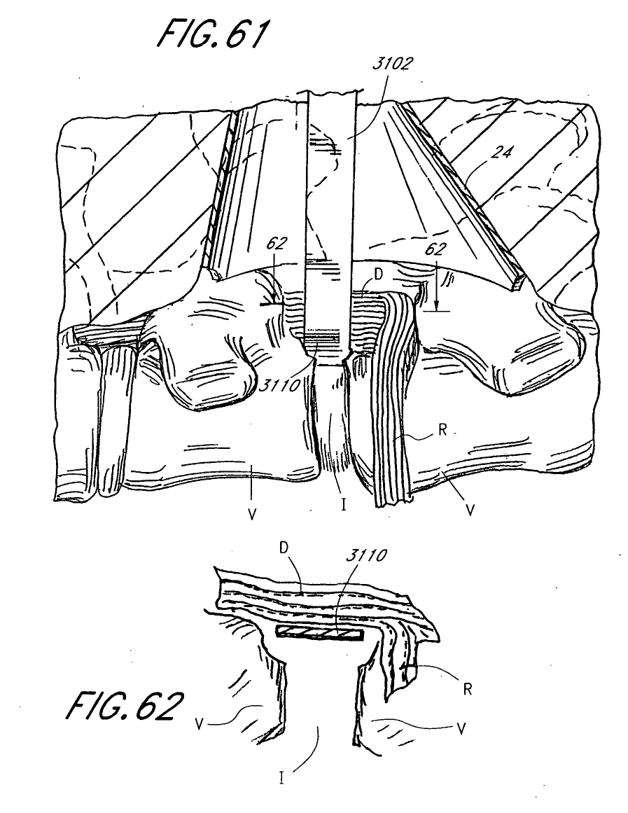

Another embodiment of the apparatus or shield is illustrated in FIGS. 63-64, and designated apparatus 3200. Apparatus 3200 is substantially identical to apparatus 3100, described above, with the following differences noted herein. In particular, distal end portion 3210 includes a pair of surfaces 3240 and 3242. Surface 3240 is an extension of elongated shield portion 3202, and surface 3242 extends at an angle with respect to surface 3240. In the exemplary embodiment, surfaces 3240 and 3242 defined an angle of about 90 degrees between them. Alternatively another angle between surfaces 3240 and 3242 may be defined as indicated by the body structures to be protected.

Distal end portion 3210 allows the apparatus to provide simultaneous shielding of both the dura D and the nerve root R. In FIGS. 65-66, surface 3242 shields the dura D, and surface 3240 shields the nerve root R. It is understood that surfaces 3240 and 3242 may be interchanged with respect to which tissue they protect during the surgical procedure.

According to the exemplary embodiment, once the fusion and fixation portions of the procedure have been performed, the procedure is substantially complete. The surgical instrumentation, such as the endoscope 500 can be withdrawn from the surgical site. The access device 20 is also withdrawn from the site. The muscle and fascia typically close as the access device 20 is withdrawn through the dilated tissues in the reduced profile configuration. The fascia and skin incisions are closed in the typical manner, with sutures, etc. The procedure described above may be repeated for the other lateral side of the same vertebrae, if indicated.

### II. Surgical Procedures that may be Performed with the Systems Described Herein

As discussed above, the systems disclosed herein provide access to a surgical location at or near the spine of a patient to enable procedures on the spine. These procedures can be applied to one or more vertebral levels, as discussed above. Additional procedures and combinations of procedures that may be performed using the systems described herein are discussed below. In various forms, these procedures involve an anterior lumbar interbody fusion, a minimally invasive lumbar interbody fusion, and other procedures particularly enabled by the access devices and systems described above.

A. Procedures Involving Anterior Lumbar Interbody Fusion

The access devices and systems described herein are amenable to a variety of procedures that may be combined with an anterior lumbar interbody fusion (referred to herein as an “ALIF”).

In one embodiment of a first method, three adjacent vertebrae, such as the L4, the L5, and the S1 vertebrae of the spine, are treated by first performing an ALIF procedure. Such a procedure may be performed in a convention manner. The ALIF involves exposing a portion of the spine, in particular the vertebrae and discs located in the interbody spaces, i.e., the spaces between adjacent vertebrae. Any suitable technique for exposing the interbody spaces may be employed, e.g., an open, mini-open, or minimally invasive procedure. In one embodiment, the interbody spaces between the L4, L5, and S1 vertebrae are exposed to the surgeon. Once exposed, the surgeon may prepare the interbody space, if needed, in any suitable manner. For example, some or all of the disc may be removed from the interbody space and the height of the interbody space may be increased or decreased. The interbody space between the L4 and the L5 vertebrae may be exposed separately from the interbody space between the L5 and S1 vertebrae or they may be generally simultaneously exposed and prepared.

After the interbody space has been exposed and prepared, a suitable fusion procedure may be performed. For example, in one example fusion procedure, one or more fusion devices may be placed in the interbody space. Any suitable fusion device may be used, e.g., a fusion cage, a femoral ring, or another suitable implant. Various embodiments of implants and techniques and tools for the insertion of implants are described in U.S. application Ser. No. 10/280,489, filed Oct. 25, 2002, which has been published as Publication No. 2003/0073998 on Apr. 17, 2003, which is hereby incorporated by reference herein in its entirety. In one variation, one or more fusion cages may be placed in an interbody space, e.g., between the L4 and L5 vertebrae, between the L5 and S1 vertebrae, or between the L4 and L5 vertebrae and between the L5 and S1 vertebrae. In another variation, one or more femoral rings may be substituted for one or more of the fusion cages and placed between the L4 and L5 vertebrae and/or between the L5 and S1 vertebrae. In another variation, one or more fusion devices are combined with a bone growth substance, e.g., bone chips, to enhance bone growth in the interbody space(s).

After anterior placement of the fusion device, an access device is inserted into the patient to provide access to a spinal location, as described above. A variety of anatomical approaches may be used to provide access to a spinal location using the access device 20. The access device preferably is inserted generally posteriorly. As used herein the, phrase “generally posteriorly” is used in its ordinary sense and is a broad term that refers to a variety of surgical approaches to the spine that may be provided from the posterior side, i.e., the back, of the patient, and includes, but is not limited to, posterior, postero-lateral, retroperitoneal, and transforaminal approaches. Any of the access devices described or incorporated herein, such as the access device 20, could be used.

The distal end of the access device may be placed at the desired surgical location, e.g., adjacent the spine of the patient with a central region of the access device over a first vertebrae. In one procedure, the distal end of the access device is inserted until it contacts at least a portion of at least one of the vertebrae being treated or at least a portion of the spine. In another procedure, the distal end of the access device is inserted until it contacts a portion of the spine and then is withdrawn a small amount to provide a selected gap between the spine and the access device. In other procedures, the access device may be inserted a selected amount, but not far enough to contact the vertebrae being treated, the portion of the vertebrae being treated, or the spine.

The access device may be configured, as described above, to provide increased access to the surgical location. The access device can have a first configuration for insertion to the surgical location over the first vertebra and a second configuration wherein increased access is provided to the adjacent vertebrae. The first configuration may provide a first cross-sectional area at a distal portion thereof. The second configuration may provide a second cross-sectional area at the distal portion thereof. The second cross-sectional area preferably is enlarged compared to the first cross-sectional area. In some embodiments, the access device may be expanded from the first configuration to the second configuration to provide access to the adjacent vertebrae above and below the first vertebra.

When it is desired to treat the L4, L5, and S1 vertebrae, the access device may be inserted over the L5 vertebrae and then expanded to provide increased access to the L4 and S1 vertebrae. In one embodiment, the access device can be expanded to an oblong shaped configuration wherein the access device provides a first dimension of about 63 mm, and a second dimension perpendicular to the first dimension of about 24 mm. In another embodiment, the access device can be expanded to provide a first dimension of about 63 mm, and a second dimension perpendicular to the first dimension of about 27 mm. These dimensions provide a surgical space that is large enough to provide access to at least three adjacent vertebrae without exposing excessive amounts of adjacent tissue that is not required to be exposed for the procedures being performed. Other dimensions and configurations are possible that would provide the needed access for procedures involving thee adjacent vertebrae.

When the access device is in the second configuration, fixation of the three vertebrae may be performed. As discussed above, fixation is a procedure that involves providing a generally rigid connection between at least two vertebrae. Any of the fixation procedures discussed above could be used in this method, as could other fixation procedures. One fixation procedure that could be used is discussed above in connection with FIG. 36 wherein the fasteners 600a, 600b, and 600c are advanced through the access device 20 to three adjacent vertebrae and are attached to the vertebrae. The three fasteners 600a, 600b, and 600c are interconnected by the elongated member 650. The three fasteners 600a, 600b, and 600c and the elongate member 650 comprise a first fixation assembly. A second fixation assembly may be applied to the patient on the opposite side of the spine, i.e., about the same location on the opposite side of the medial line of the spine. Other fixation procedures could be applied, e.g., including two fasteners that coupled to the L4 and the S1 vertebrae and all elongate member interconnecting these vertebrae.

One variation of the first method provides one level of fixation on the anterior side of the patient, e.g., when the fusion device is placed in the interbody space. For example, fixation of the L5 and S1 vertebrae could be provided on the anterior side of the spine, in addition to the other procedures set forth above (e.g., a two level postero-lateral fixation). Also, fixation of the L4 and L5 vertebrae could be provided on the anterior side of the spine, in addition to the other procedures set forth above (e.g., a two level postero-lateral fixation).

In a second method, substantially the same steps as set forth above in connection with the first method would be performed. In addition, after the access device is inserted, a decompression procedure is performed through the access device. A decompression procedure is one where unwanted bone is removed from one or more vertebrae. Unwanted bone can include stenotic bone growth, which can cause impingement on the existing nerve roots or spinal cord. Decompression procedures that may be performed include laminectomy, which is the removal of a portion of a lamina(e), and facetectomy, which is the removal of a portion of one or more facets. In one variation of this method, decompression includes both a facetectomy and a laminectomy. Any suitable tool may be used to perform decompression. One tool that is particularly useful is a kerrison.

In a third method, substantially the same steps as set forth above in connection with the first method would be performed. That is, an ALIF procedure is performed in combination with a fixation procedure. In addition, a fusion procedure may be performed through the access device which may have been placed generally posteriorly, e.g., postero-laterally, tranforaminally or posteriorly, whereby bone growth is promoted between the vertebrae and the fixation assembly, including at least one of the fasteners 600a, 600b, 600c and/or the elongate element 650. This procedure is also referred to herein as an “external fusion” procedure.

One example of an external fusion procedure that may be performed involves placement of a substance through the access device intended to encourage bone growth in and around the fixation assembly. Thus, fusion may be enhanced by placing a bone growth substance adjacent any of the fasteners 600a, 600b, 600c and/or the elongate member 650. The bone growth substance may take any suitable form, e.g., small bone chips taken from the patient (e.g., autograft), from another donor source (e.g., allograft or xenograft), and orthobiologics.

After the bone growth substance is applied to the fixation assembly, the access device is removed. Absent the retracting force provided by the access device, the patient's tissue generally collapses onto the bone growth substance. The tissue will thereby maintain the position of the bone growth substance adjacent to the fixation assembly. The presence of the bone growth substance can cause bone to bridge across from the vertebra(e) to one or more components of the fixation assembly.

In a fourth method, substantially the same steps as set forth above in connection with the second method would be performed. That is, an ALIF procedure is performed anteriorly, and a decompression procedure and a fixation procedure are performed through the access device which may be placed generally posteriorly, e.g., postero-laterally, tranforaminally, or posteriorly. In addition, bone growth substance is placed in and around a fixation assembly through the access device, as discussed above in connection with the third method. The bone growth substance encourages bone to bridge across from the vertebrae to the fixation assembly.

In a fifth method, an ALIF procedure is performed, as discussed above in connection with the second method. After one or more fusion devices is placed in the interbody space, access is provided by way of the access device, as discussed above, from any suitable anatomical approach, e.g., a generally posterior approach. Preferably, a postero-lateral approach is provided. After access has been provided, a bone growth substance, such as those discussed above in connection with the third method, is delivered through the access device. The bone growth substance is placed adjacent an interbody space, e.g., the space between the L4 and the L5 vertebrae and/or between the L5 and the S1 vertebrae. The bone growth substance encourages fusion of the adjacent vertebrae, e.g., L4 to L5 and/or L5 to S1, by stimulating or enhancing the growth of bone between adjacent vertebrae, as discussed above.

In a sixth method, substantially the same steps described in connection with the first method are performed, except that the fixation procedure is optional. In one variation of the sixth method, the fixation procedure is not performed. However, after the access device is inserted, a bone growth substance is placed in and around one or more interbody spaces through the access device. Where the sixth method involves a two level procedure, the bone growth substance can be placed adjacent the interbody space between the L4 and the L5 vertebra and/or between the L5 and the S1 vertebra. Thus, bone growth may occur in the interbody space and adjacent the interbody space between the vertebrae.

The foregoing discussion illustrates that an ALIF procedure can be combined with a variety of procedures that can be performed through an access device disclosed herein. In addition, though not expressly set forth herein, any combination of the procedures discussed above, and any other suitable known procedure, may also be combined and performed through the access devices described herein, as should be understood by one skilled in the art.

B. Spine Procedures Providing Minimally Invasive Lumbar Interbody Fusion

Another category of procedures that may be performed with the access devices and systems described above involves a minimally invasive lumbar interbody fusion (referred to herein as a “MILIF”). MILIF procedures are particularly advantageous because they permit the surgeon to perform a wide variety of therapeutic procedures without requiring fusion by way of an anterior approach, as is required in an ALIF. This provides a first advantage of allowing the surgeon to perform all procedures from the same side of the patient and also possibly from the same approach. Also, the access devices and systems disclosed herein provide the further advantage of enabling two level procedures, and many other related procedures, to be performed by way of a single percutaneous access. These and other advantages are explained more fully below.

In a first MILIF method, a two level postero-lateral fixation of the spine involving three adjacent vertebrae, such as the L4, L5, and S1 vertebrae, is provided. Analogous one level procedures and two level procedures involving any other three vertebrae also may be provided. In addition, the access devices and systems described herein could be used or modified to accommodate other multi-level procedures, such as a three level procedure. The surgeon inserts an access device such as described herein to a surgical location near the spine. As discussed above, the access devices are capable of a wide variety of anatomical approaches. In this procedure, a postero-lateral approach is preferred. Once the access device is inserted to a location adjacent the spine, as discussed above, it may be configured, e.g., expanded, as discussed above, to a configuration wherein sufficient access is provided to the surgical location.

Any suitable fusion process may then be performed. For example, an implant may be advanced through the access device into the interbody space in order to maintain disc height and allow bone growth therein, e.g., as in a fusion procedure. In order to ease insertion of the implant, it may be beneficial to prepare the interbody space. Interbody space preparation may involve removal of tissue or adjusting the height of the interbody space through the access device, such as in a distraction procedure. Once the interbody space is prepared, a suitable implant may be advanced through the access device into the interbody space, taking care to protect surrounding tissues. Various embodiments of implants and techniques and tools for their insertion are described in U.S. application Ser. No. 10/280,489, incorporated by reference hereinabove. In general, the implant preferably is an allograft strut that is configured to maintain disc height and allow bone growth in the interbody space.

In addition to providing a suitable fusion, the first method provides fixation of the vertebrae. The fixation procedure may take any suitable form, e.g., ally of the fixation procedures similar to those disclosed above. In particular, when the access device is in the expanded or enlarged configuration, fixation of the three adjacent vertebrae may be performed. One fixation procedure that could be used is discussed above in connection with FIG. 36 wherein the fasteners 600a, 600b, and 600c are advanced through the access device 20 to three adjacent vertebrae and are attached to the vertebrae. The three fasteners 600a, 600b, and 600c are interconnected by way of the elongated member 650. As discussed above, a second fixation assembly may be applied to the patient on the opposite side of the spine, e.g., about the same location on the opposite side of the medial line of the spine.

In a second MILIF method, substantially the same procedures set forth above in connection with the first MILIF method are performed. In addition, a suitable decompression procedure may be performed, as needed. As discussed above, decompression involves removal of unwanted bone by way of a suitable decompression technique that may be performed through the access device. In one embodiment, decompression is performed through the access device after the access device has been expanded. As discussed above, suitable decompression techniques include a laminectomy, a facetectomy, or any other similar procedure. Decompression for the L4, the L5, and/or the S1 vertebrae may be needed and can be performed through the access devices described herein without requiring the access device to be moved from one position to another.

In a third MILIF method, substantially the same procedures set forth above in connection with the first MILIF method are performed. In addition, a further fusion procedure, e.g., a fusion procedure external to the interbody space, is provided. The external fusion procedure is performed adjacent to the interbody space wherein bone growth may be promoted in the proximity of the fixation assembly, e.g., above the postero-lateral boney elements of the spine, such as the facet joints and the transverse processes. In one embodiment, when the fixation assembly comprising the fasteners 600a, 600b, 600c and/or the elongate element 650 has been applied to three adjacent vertebrae, a substance is applied through the access device to one or more components of the fixation assembly to maintain or enhance the formation and/or growth of bone in the proximity of the fixation assembly. For example, a bone growth substance may be placed adjacent any of the fasteners 600a, 600b, 600c and/or the elongate member 650. Bone growth substance may take any suitable form, e.g., small bone chips taken from the patient (e.g., autograft), from another donor source (e.g., allograft or xenograft), and orthobiologics.

After the bone growth substance is applied to the fixation assembly, the access device is removed. Absent the retracting force provided by the access device, the patient's tissue generally collapses onto the bone growth substance. The tissue will thereby maintain the position of the bone growth substance adjacent to the fixation assembly. The presence of the bone growth substance advantageously causes bone to grow between the vertebrae and the fixation assembly to form a bridge therebetween.

A fourth MILIF method involves substantially the same procedures performed in connection with the third MILIF method. In particular, one or more implants are positioned in the interbody spaces through an access device, a fixation procedure is performed through the access device, and a further fusion procedure is preformed wherein bone growth substance is positioned adjacent the interbody space through the access device. In addition, a decompression procedure is performed through the access device that may include a facetectomy and/or a laminectomy.

A fifth MILIF method involves substantially the same procedures performed in connection with the first MILIF method, except that the fixation is optional. In one embodiment, the fixation is not performed. In addition, a further fusion procedure is performed through the access device wherein bone growth substance is positioned adjacent the interbody space, as discussed above.

A sixth MILIF method is substantially the same as the fifth MILFF method, except that a further fusion procedure is performed through the access device. In particular, an implant is positioned in the interbody space through an access device, a decompression procedure is performed through the access device, and a further fusion procedure is performed whereby bone growth substance is placed adjacent the interbody space through the access device. As discussed above, the decompression procedure may include a facetectomy, a laminectomy, and any other suitable procedure. As with any of the methods described herein, the procedures that make up the sixth MILIF method may be preformed in any suitable order. Preferably the decompression procedure is performed before the external fusion procedure.

The foregoing discussion illustrates that a MILIF procedure can include a variety of procedures that can be performed through an access device described herein. In addition, though not expressly set forth herein, any combination of the procedures discussed above, and any other suitable known procedures, may also be combined, as should be understood by one skilled in the art.

C. Other Multi-Level Procedures

While the foregoing procedures have involved interbody fusion, the access devices and systems described herein can be employed in a variety of single level and multi-level procedures (e.g., more than two levels) that do not involve an interbody fusion. For example, a discectomy can be performed through the access devices described herein without implanting an interbody fusion device thereafter, e.g., to remove a herneation. In another embodiment, a discectomy can be performed in more than one interbody space without inserting an interbody fusion device into each interbody space, e.g., to remove multiple herneations. In another embodiment, a single or multi-level decompression procedure can be performed to remove unwanted bone growth.

It will be understood that the foregoing is only illustrative of the principles of the invention, and that various modifications, alterations, and combinations can be made by those skilled in the art without departing from the scope and spirit of the invention. Some additional features and embodiments are described below.

### III. Additional Features and Embodiments of Systems and Methods for Performing Surgical Procedures

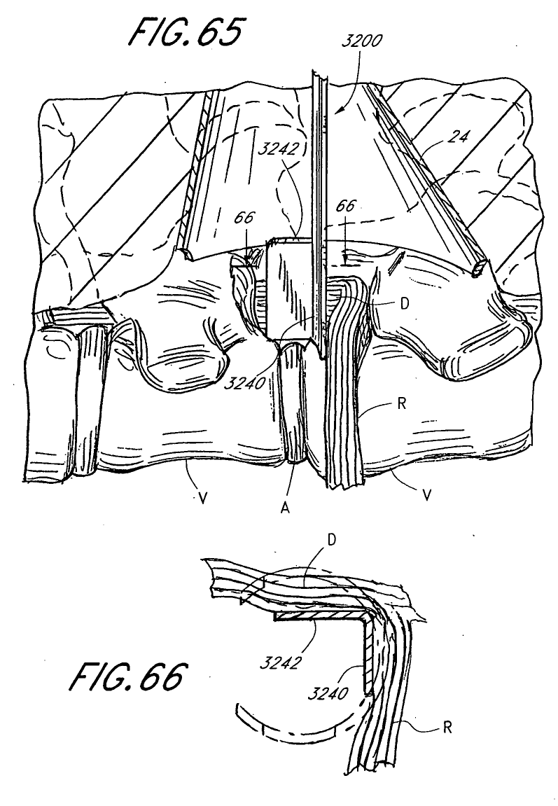

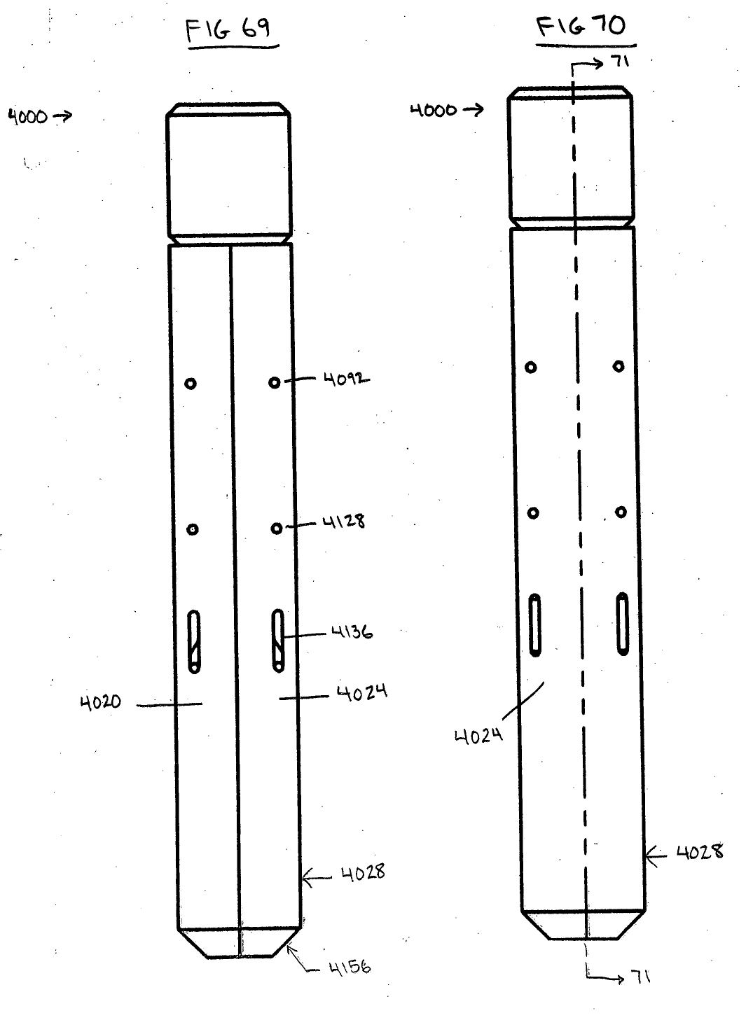

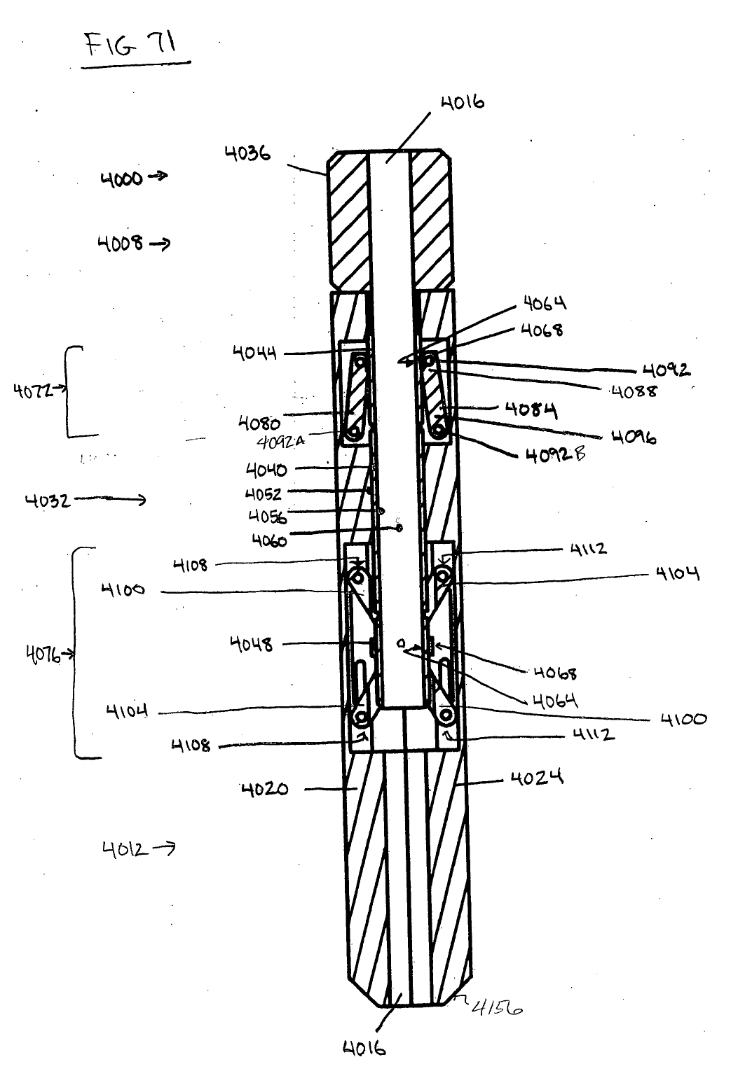

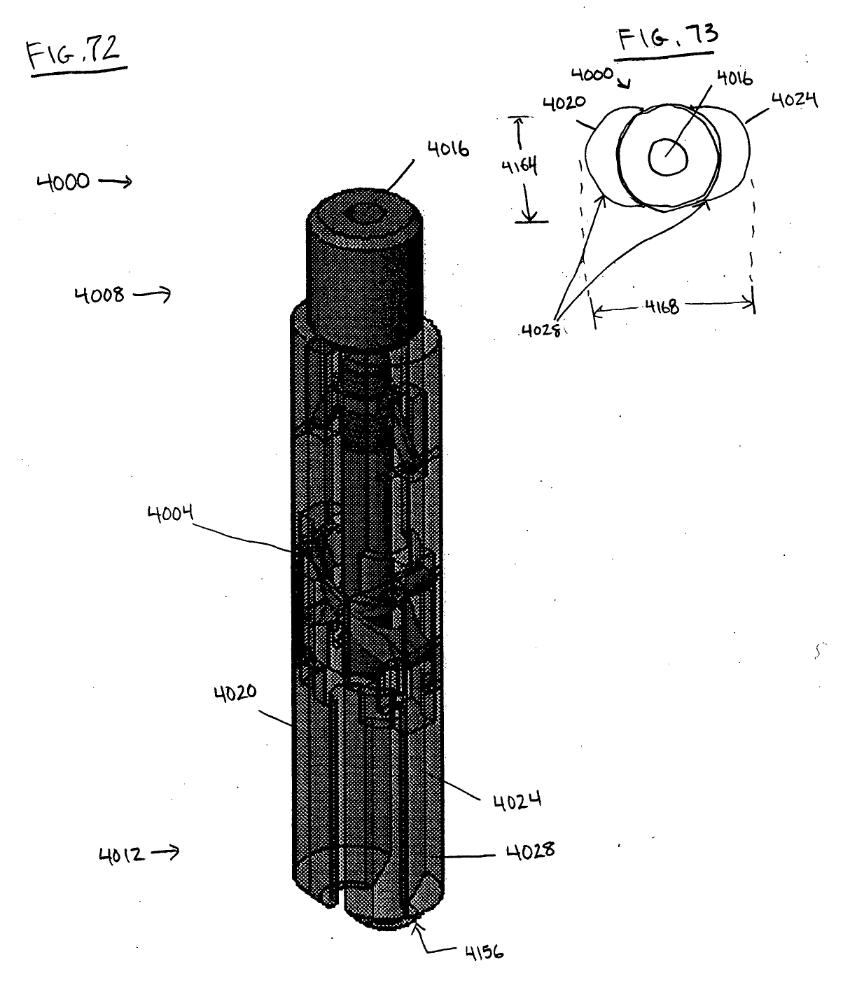

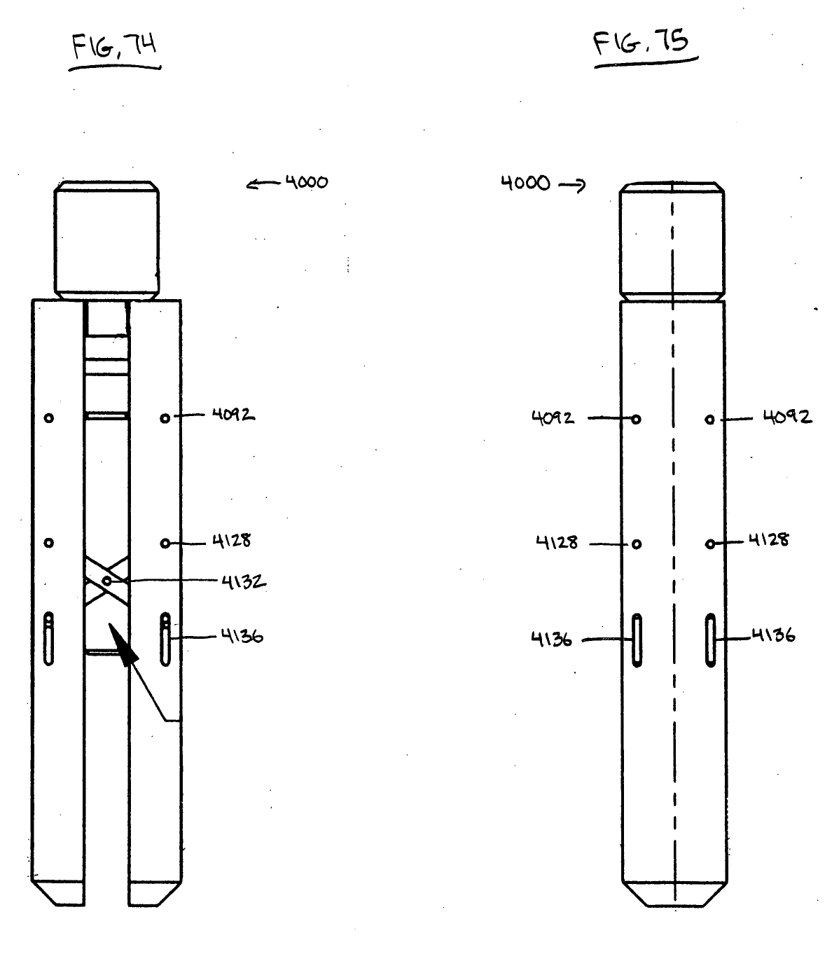

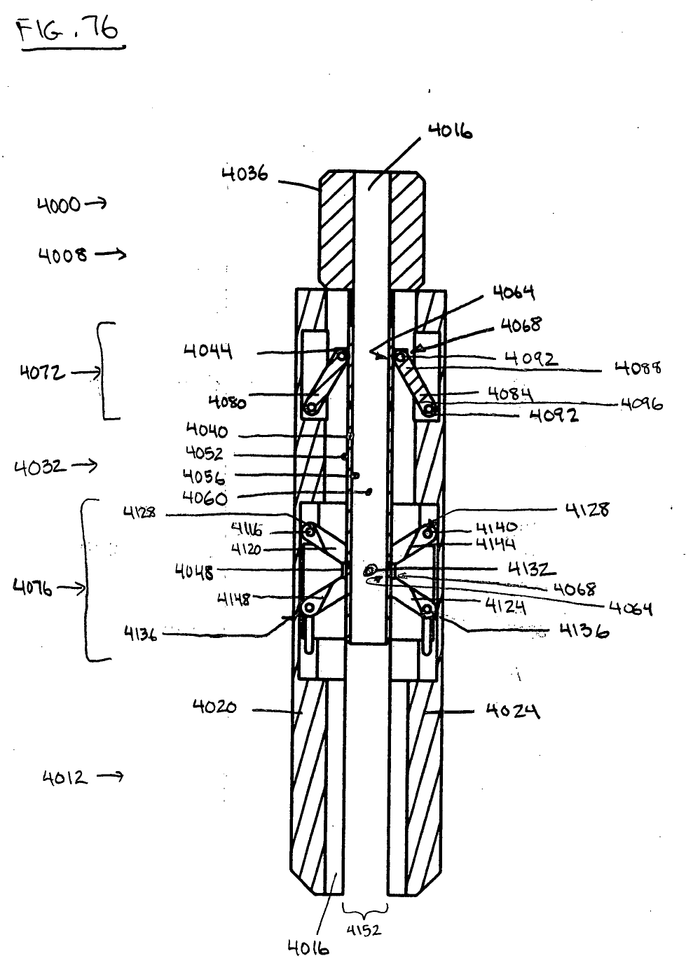

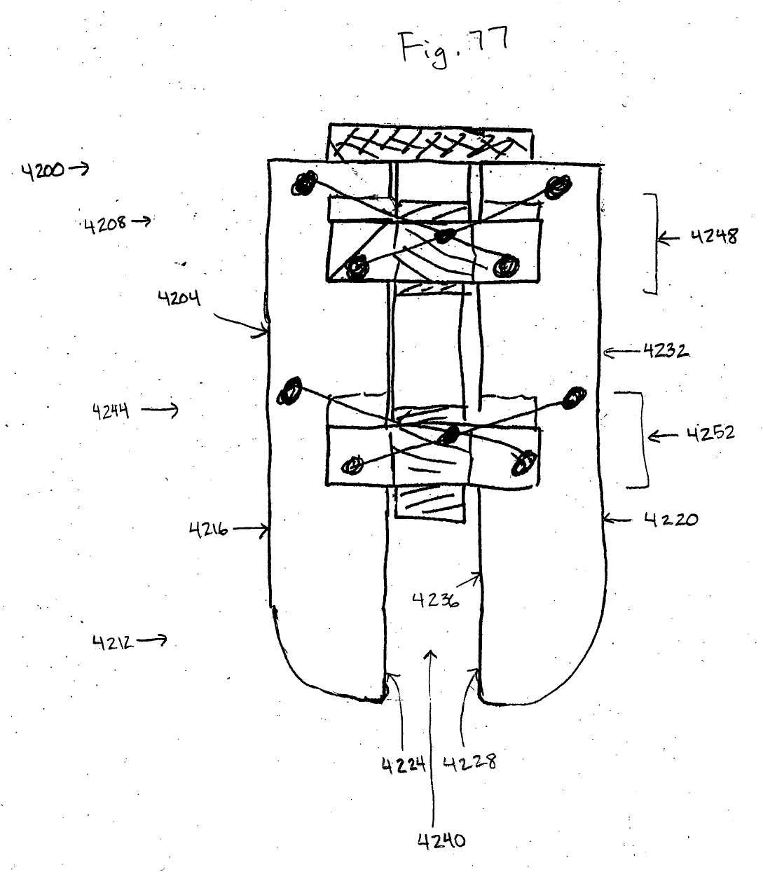

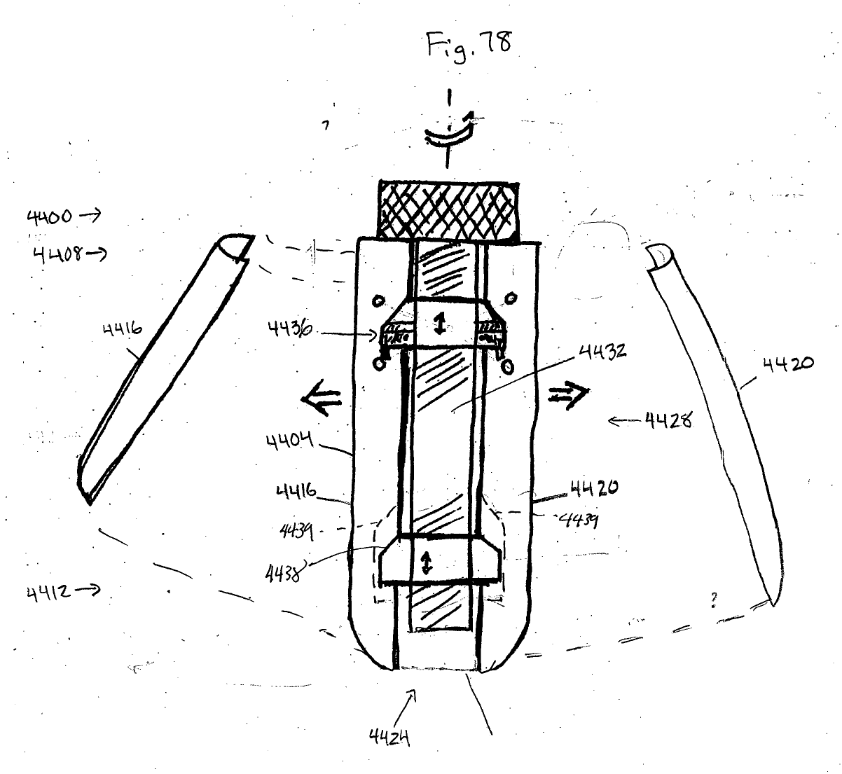

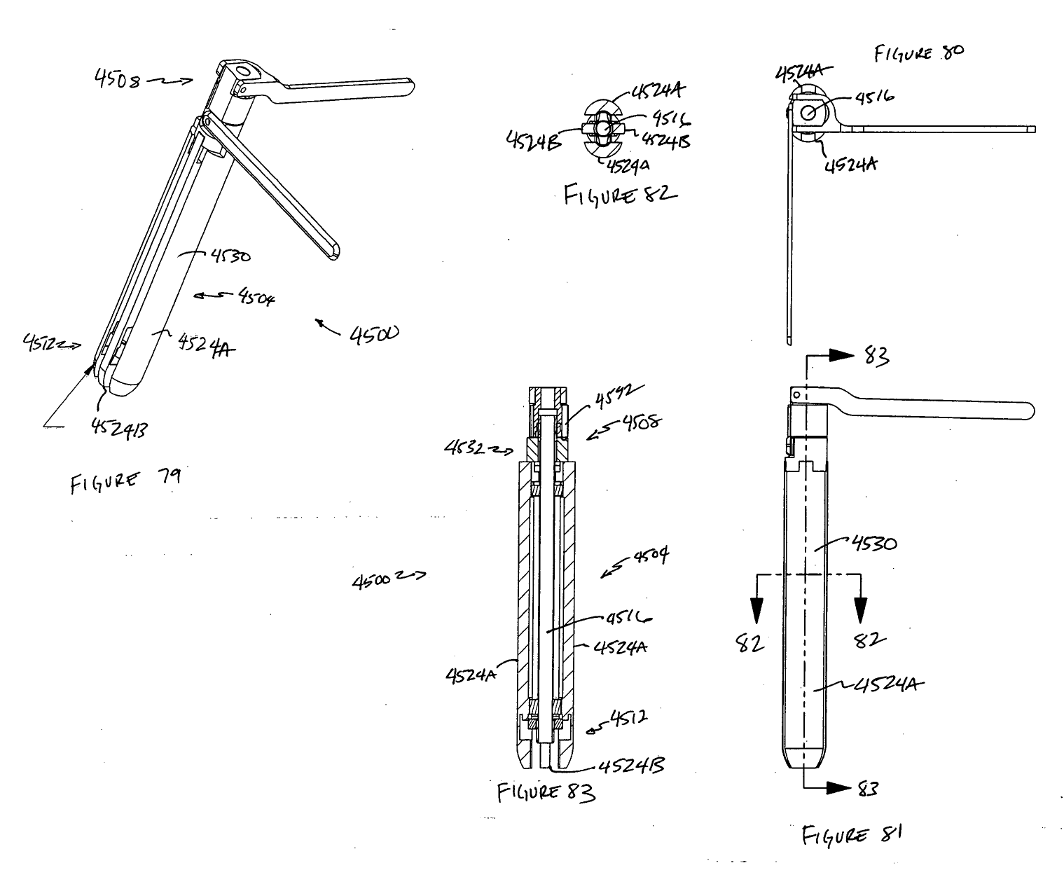

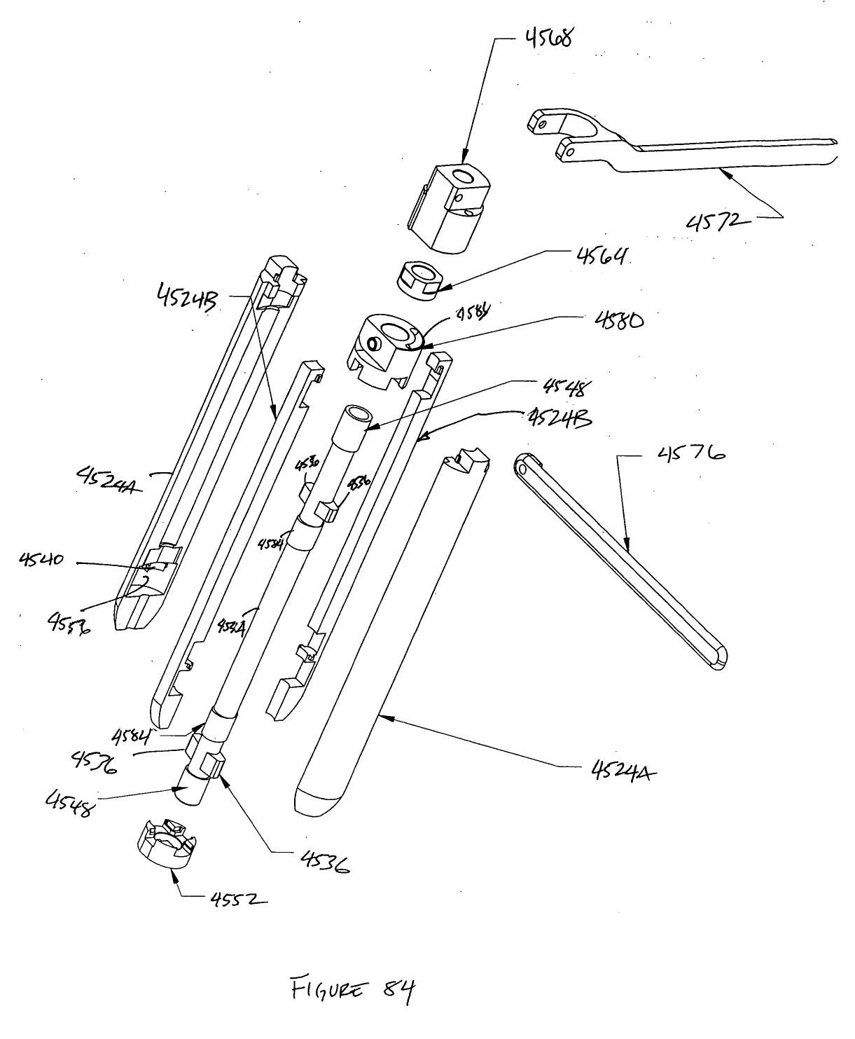

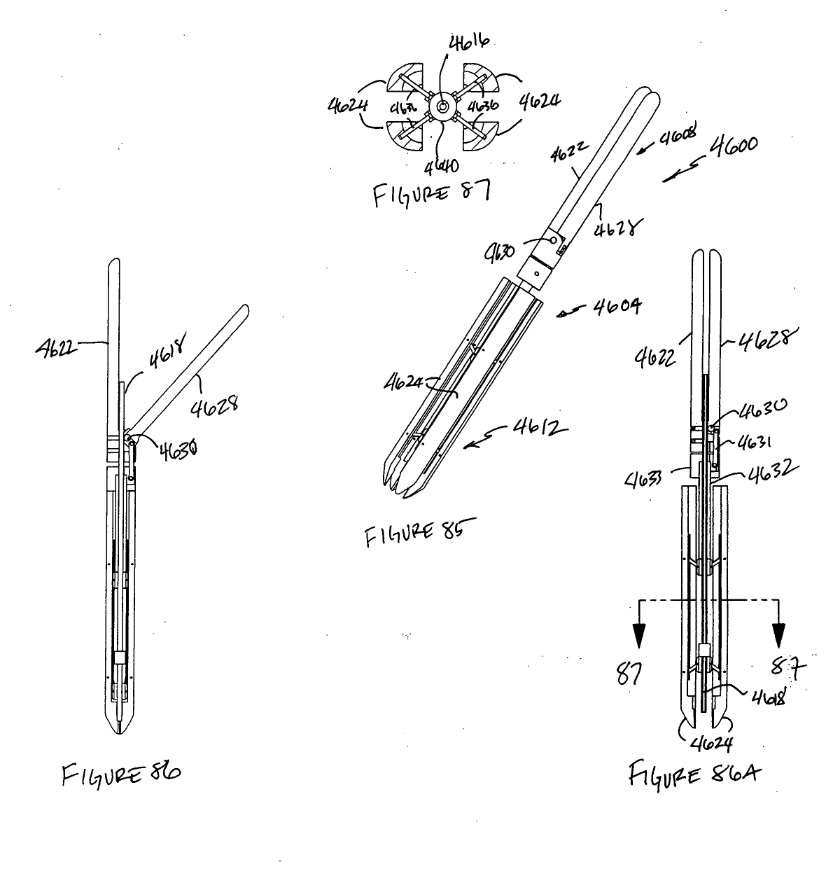

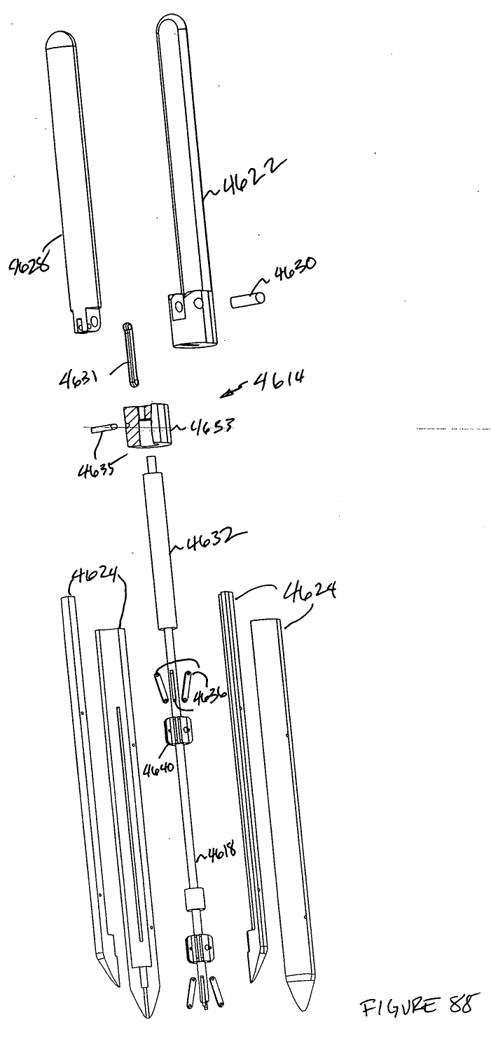

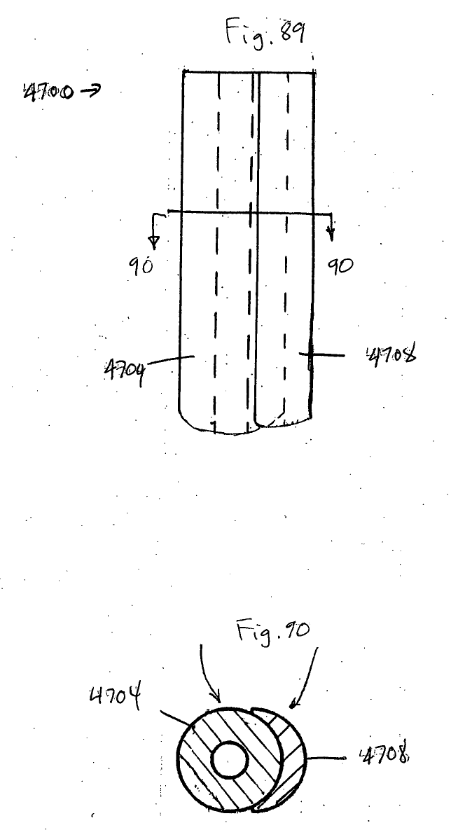

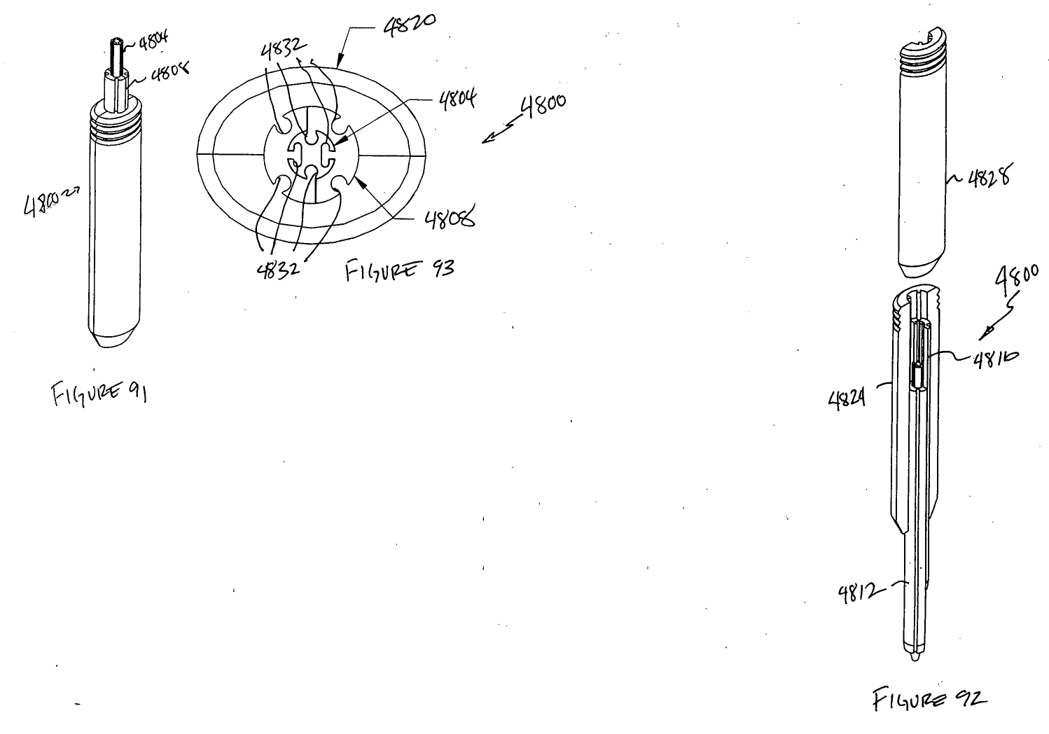

A variety of embodiments of dilator apparatuses, systems, assemblies, and techniques will now be further discussed. These embodiments generally include expandable dilating structures, which enable a comparatively small transverse profile dilator to be expanded to a transverse profile suitable for insertion thereover of an access device, such as any of the access devices described or incorporated herein by reference. In some cases, the expansion of the dilator embodiments transforms the cross-sectional profile thereof from an axisymmetric, e.g., a round, profile to an oblong or other asymmetrical profile. FIGS. 67-88 illustrate dilator embodiments that include an actuation system, e.g., including at least one mechanism, to move portions of the dilator to increase the transverse profile thereof. FIGS. 89-93 illustrate dilator assembly embodiments that are at least partially assembled within the patient to increase the transverse profile of a dilated passage to a surgical location.

With reference to FIGS. 67-76, in one embodiment, a dilator 4000 is adapted to dilate or retract tissue at a region of the spine. As discussed further below, the dilator 4000 is configured to expand the size of an incision and is particularly advantageous for use in a minimally invasive procedure, such as any of those described herein. As discussed further below, the dilator 4000 can be sized to extend from a position above the incision (e.g., outside the patient) to a location adjacent a patient's spine. As such, the dilator can be used to dilate or retract subcutaneous tissue beneath the incision to expand or define a passage through the tissue. As discussed further below, the dilator 4000 may be used to facilitate the insertion of an access device or retractor into a patient to facilitate such procedures. The dilator 4000 is shown in a low profile configuration in FIG. 67 and in an expanded configuration in FIG. 72. The dilator 4000 preferably comprises an elongate body 4004 having a proximal portion 4008 and a distal portion 4012.

In some embodiments, the elongate body 4004 defines a dilator passage 4016 extending between the proximal portion 4008 and the distal portion 4012, e.g., from a proximal end of the proximal portion 4008 to a distal end of the distal portion 4012. In other embodiments, the elongate body 4004 can be solid. As discussed further below, the dilator passage 4016 can be configured to receive a smaller structure that can be inserted though the incision. For example, some procedures involve insertion of the dilator 4000 through the incision along a guidewire that was previously advanced through the incision. Accordingly, the passage 4016 can be configured, e.g., sized, to receive a guidewire or can be configured such that the dilator 4000 can be advanced over a guidewire.

In one arrangement, the dilator 4000 comprises a first elongate member 4020 and a second elongate member 4024. In some embodiments, the first elongate member 4020 is a first blade and the second elongate member 4024 is a second blade. In this context, the use of the term “blade” is intended to be a broad term including generally thin elongate structures that have sufficient stiffness to push tissue aside, and includes structures that are flat or curved in transverse cross-section. At least one of the first elongate member 4020 and the second elongate member 4024 defines an outer surface 4028 of the dilator 4000. In one arrangement, the first and second elongate members 4024, 4028 define the outer surface 4028. In one embodiment a transverse cross-section of at least the distal portion 4014 of the dilator 4000 defines a substantially continuous perimeter, e.g., a substantially continuous circular perimeter.

As discussed further below, the dilator 4000 preferably is expandable. In one embodiment, the dilator 4000 is actuatable between a low profile configuration and an expanded configuration. Various techniques can be provided for expanding an expandable dilator. FIG. 67 shows that in one embodiment, the dilator includes discrete proximal and distal portions 4008, 4012. The proximal portion 4008 can be configured as a dilator expansion device. In some cases, as discussed further below, expansion devices operate by actuating a mechanism that is at least partially housed within the elongate body 4004, e.g., in the distal portion 4012. For example as discussed below, the proximal portion 4008 can be arranged to be rotatable about a longitudinal axis 4030 that extends through a central portion of the dilator 4000, e.g., through the center of the dilator passage 4016. Rotation of the proximal portion 4008 relative to the distal portion 4012 is one technique for actuating the dilator 4000, as discussed further below.

More particularly, as shown in FIGS. 71 and 76, in one embodiment the dilator 4000 comprises an actuation system 4032. The first elongate member 4020 and the second elongate member 4024 are coupled to the actuation system 4032. The actuation system 4032 comprises one or more actuation elements that are configured to move within the dilator 4000 to facilitate expansion of the dilator. In some embodiments, the actuation elements can include one or more members that extend between the actuation element and at least one of the first and second elongate members 4024, 4208. In the illustrated embodiment, the actuation system 4032 comprises a knob 4036, a shaft 4040, a first coupling member 4044, and a second coupling member 4048. At least one of the first and second coupling member 4044, 4048 is configured to move within the dilator. For example, at least one of the first and second coupling member 4044, 4048 is configured to translate within the dilator 4000 between at a first position and a second position, the second position being distal to the first position. In one embodiment, the first position is between the proximal portion 4008 and the second position. FIG. 71 illustrates the first and second coupling member 4044, 4048 in the first position. FIG. 76 illustrates the first and second coupling member 4044, 4048 in the second position. In the illustrated embodiment, the first coupling member 4044 and the second coupling member 4048 are rings having internal threads and external couplings whereby linkages can be attached thereto, as discussed further below. Where the first and second couplings 4044, 4048 are threaded, the shaft 4040 includes external threads configured to mate with the threads of the couplings. The threads on the shaft 4040 extend at least a length sufficient to provide enough travel of the couplings to provide the amount of expansion of the dilator 4000 needed for the particular application, as discussed below.

The knob 4036 preferably is located at a proximal portion 4008 of the dilator 4000. The knob 4036 is coupled with a proximal portion of the shaft 4040. Rotation of the knob 4036 preferably rotates the shaft 4040. The knob 4036 can be manually rotated by an operator, or it can be driven by any number of automated mechanisms known in the art. Where the rotation of the know 4036 is intended to be manual, it may be desirable to provide an external surface that is configured to enhance the grippability of the knob, e.g., a knurled surface.

In the illustrated embodiment, the shaft 4040 has an outer surface 4052, an inner surface 4056, and a bore 4060 defined therethrough. The bore 4060 and the shaft 4040 corresponds to a portion of the dilator passage 4016. The bore 4060 preferably is sized to receive or to be advanced over a guide pin or guidewire for insertion into a patient. In other arrangements, the shaft 4040 does not include a bore 4060, e.g., with dilating procedures that do not benefit from the pre-placement of a guide pin or wire.

As discussed above, the outer surface 4052 of the shaft 4040 preferably is configured to cooperate with at least one of the first coupling member 4044 and the second coupling member 4048. For example, the outer surface 4052 can be configured to enable at least one of the coupling members 4044, 4048 to translate therealong. In the illustrated embodiment, the outer surface 4052 of the shaft 4040 is threaded along a length of the shaft 4040, as discussed above. The threading of the coupling members 4044, 4048 and the shaft 4040 enables controlled operation of the actuation system 4032 and relatively precise expansion of the dilator 4000, as discussed below.

As shown in FIGS. 71 and 76, the first coupling member 4044 and the second coupling member 4048 each have an inner surface 4064 and an outer surface 4068. The inner surface 4056 of the coupling members is threaded in the illustrated embodiment. The threads of the coupling members preferably cooperate with the threads of the shaft so that turning the shaft 4040 with the knob 4036 causes the first coupling member 4044 and the second coupling member 4048 to move relative to the shaft 4040. For example, as the shaft 4040 is rotated, the first coupling member 4044 and the second coupling member 4048 will move up or down along the shaft 4040. The first coupling member 4044 and the second coupling member 4048 can move in the same direction in some embodiments. In the illustrated embodiment, the first coupling member 4044 and the second coupling member 4048 move in different directions when the shaft 4040 is rotated, as described below.

The outer surface 4068 of the first coupling member 4044 and the second coupling member 4048 preferably are coupled to the first elongate member 4020 and the second elongate member 4024 of the dilator 4000. More particularly, as shown in FIGS. 71 and 76, the first coupling member 4044 and the second coupling member 4048 are coupled to the first elongate member 4020 and the second elongate member 4024 via linkages in one embodiment. In the illustrated embodiment, a first linkage 4072 is coupled between the first coupling member 4044, the first elongate member 4020, and the second elongate member 4024. A second linkage 4076 is coupled between the second coupling member 4048, the first elongate member 4020, and the second elongate member 4024 in the illustrated embodiment.

The first linkage 4072 has a first link element 4080 and a second link element 4084. The first link element 4080 and the second link element 4084 each have first ends 4088 coupled to opposite sides of the first coupling member 4044 via pin connections 4092. The first link element 4080 has a second end 4196A coupled to the first elongate member 4020 via a pin connection 4092 and the second link element 4084 has a second ends 4196B coupled to the second elongate member 4024 via a pin connections 4092.

The first linkage 4072 is actuated by rotation of the knob 4036 in the illustrated embodiment. In one technique, the knob 4036 is rotated clockwise, as viewed from the proximal end, which rotation causes the first coupling member 4044 to move distally along the shaft 4040. The distal movement of the first coupling member 4044 actuates the first linkage 4072. In one arrangement, the distal movement of the first coupling member 4044 applies a force to the pin connection 4092 between the first link element 4080 and the first coupling member 4044. This force is transmitted through the first link element 4080 to the pin connection 4092A, causing the first link element 4080 to rotate outward from the shaft 4040. The second link element 4084 is similarly actuated by the movement of the first coupling member 4044. The operation of the actuation system transmits mechanical force from the knob 4036 through first linkage 4072 to a proximal portion of the first and second elongate elements 4024, 4028 to push the first and second elongate elements outward from the shaft 4040 to enable dilation of tissue, as discussed further below.

In some embodiments, the second linkage 4076 is provided to additional transmit some of the force applied to the knob 4036 to a distal portion of the first and second elongate member 4024, 4028. In one arrangement, the second linkage 4076 has a first crosslink element 4100 and a second crosslink element 4104. The first crosslink element 4100 and the second crosslink element 4104 each have a first side portion 4108 and a second side portion 4112. The first side portion 4108 of the first crosslink element 4100 is positioned on an opposite sides of the shaft 4040 and the second coupling member 4048 from the first side portion 4108 of the second side portion 4112. The second side portion 4112 of the first crosslink element 4100 is positioned on an opposite sides of the shaft 4040 and the second coupling member 4048 from the second side portion 4112 of the second side portion 4112. The first crosslink element 4100 and the second crosslink element 4104 each have a fixed end portion 4116, an intermediate portion 4120, and a translating end portion 4124.

The fixed end portion 4116 of the first crosslink element 4100 is coupled to the first elongate member 4020 at a fixed pin location 4128 on the first elongate member 4020. The intermediate portion 4120 of the first crosslink element 4100 is coupled to the second coupling member 4048 via pin connections 4132 on either side of the second coupling member 4048. The translating end portion 4124 of the first crosslink element 4100 is coupled to the second elongate member 4024 at a translating pin location 4136 on the second elongate member 4024.

The fixed end portion 4140 of the second crosslink element 4104 is coupled to the second elongate member 4024 at a fixed pin location 4128 on the second elongate member 4024. The intermediate portion 4144 of the second crosslink element 4104 is coupled to the second coupling member 4048 via pin connections 4132 on either side of the second coupling member 4048. The translating end portion 4148 of the second crosslink element 4104 is coupled to the first elongate member 4020 at a translating pin location 4136 on the first elongate member 4020. The translating pin locations 4136 on the first and second elongate members 4016, 4020 can comprise elongate slots in the members along which a pin can translate to enable actuation of the second linkage 4076.

In the illustrated embodiment of FIGS. 67-76, the first linkage 4072 and the second linkage 4076 are configured to be actuated by opposing movement of portions of the actuation system 4032. For example, the actuation system 4032 can be arranged such that the first coupling member 4044 and the second coupling member 4048 translate in opposite directions to enable the expansion of the dilator 4000. Preferably the opposing motion is achieved substantially simultaneously by a single motion of the knob 4036. In one embodiment, the first coupling member 4044 has a first threaded portion on the inner surface, e.g. a left hand thread portion, and the second coupling member 4048 has a second type of threaded portion on the inner surface, e.g. a right hand thread portion. Accordingly, as the knob 4036 and threaded shaft 4040 are rotated, the first coupling member 4044 and the second coupling member 4048 will move along the shaft 4040 in opposing or different directions. In one arrangement, as the knob 4036 is turned clockwise as viewed from the proximal end of the dilator 4000, the first coupling member 4044 and the second coupling member 4048 will move away from each other causing the dilator 4000 to move to the un-expanded configuration. For example, as the knob 4036 is turned clockwise, the first coupling member 4044 moves up the shaft 4040, while the second coupling member 4048 moves down the shaft. FIG. 71 illustrates a low profile or un-expanded configuration of the dilator 4000. In one arrangement, as the knob 4036 is turned counter-clockwise as viewed from the proximal end of the dilator 4000, the first coupling member 4044 and the second coupling member 4048 will move toward each other causing the dilator 4000 to move to the expanded configuration. For example, as the knob 4036 is turned counter-clockwise, the first coupling member 4044 down the shaft 4040, while the second coupling member 4048 moves up the shaft. FIGS. 72-76 illustrate expanded configurations configuration of the dilator 4000.

As the first coupling member 4044 and the second coupling member 4048 travel along the shaft 4040, the first linkage 4072 and the second linkage 4076 act to expand or contract the distance between the first elongate/member 4020 and the second elongate member 4024 of the dilator 4000. In the illustrated embodiment, a gap 4152 or space is created between the first elongate member 4020 and the second elongate member 4024 when the dilator 4000 is in the expanded configuration. As the gap 4152 is being enlarged, tissue adjacent to the first elongate member 4020 and the second elongate member 4024 of the dilator 4000 is being dilated and a passage between the incision in the skin and a spinal location is being expanded.

The length of the dilator 4000 can be any suitable length as is convenient for the procedure to be performed and for the individual size and configuration of the patient's anatomy. The length of the dilator 4000 be preferably is between about 30 mm and about 110 mm for some applications. In some embodiments, the overall length of the dilator 4000 preferably is between about 50 mm and about 80 mm for other applications. In some embodiments, the overall length of the dilator 4000 preferably is between about 60 mm and about 70 mm for other applications. The dilator 4000, and any of the other dilators described herein, can have features that aid in accurate insertion. Such features can include depth marks. Depth marks can take any suitable form. For example, depth marks can be configured as a plurality of lines at regular increments. For example, in one embodiment, the dilator 4000 is about 150 millimeters long and depth marks are formed on the dilator at 10 millimeter increments starting at 40 millimeters from the distal end and ending at 110 millimeters from the distal end.

The elongate body 4004 can have any suitable shape. In one embodiment, the distal portion 4012 of the elongate body 4004 is tapered adjacent a distal end thereof. See FIGS. 71-72. The tapered distal portion 4156 of the dilator 4000 is configured to retract tissue as the dilator 4000 is advanced into the patient. In one embodiment, the cross-sectional shape of the distal portion 4012 of the elongate body 4004 and of the retractor 4000 is oblong. See FIG. 73. The cross-sectional shape of the corresponding opening in the tissue defined by the elongate body 4004 at one location also is oblong in one technique. In some embodiments, the cross-sectional shape of the opening in the tissue defined by the elongate body 4004 at one location can be oval, elliptical, rectangular, circular, rounded, square, or other shapes or combinations of shapes. These shapes can be configured to correspond with the shape of an access device, such as any of those described or incorporated by reference herein. As discussed further below, by expanding the dilator 4004 to an enlarged size and (in some techniques) a non-circular transverse cross-sectional shape, an access device with a non-circular transverse cross-section can be more easily inserted into the patient.

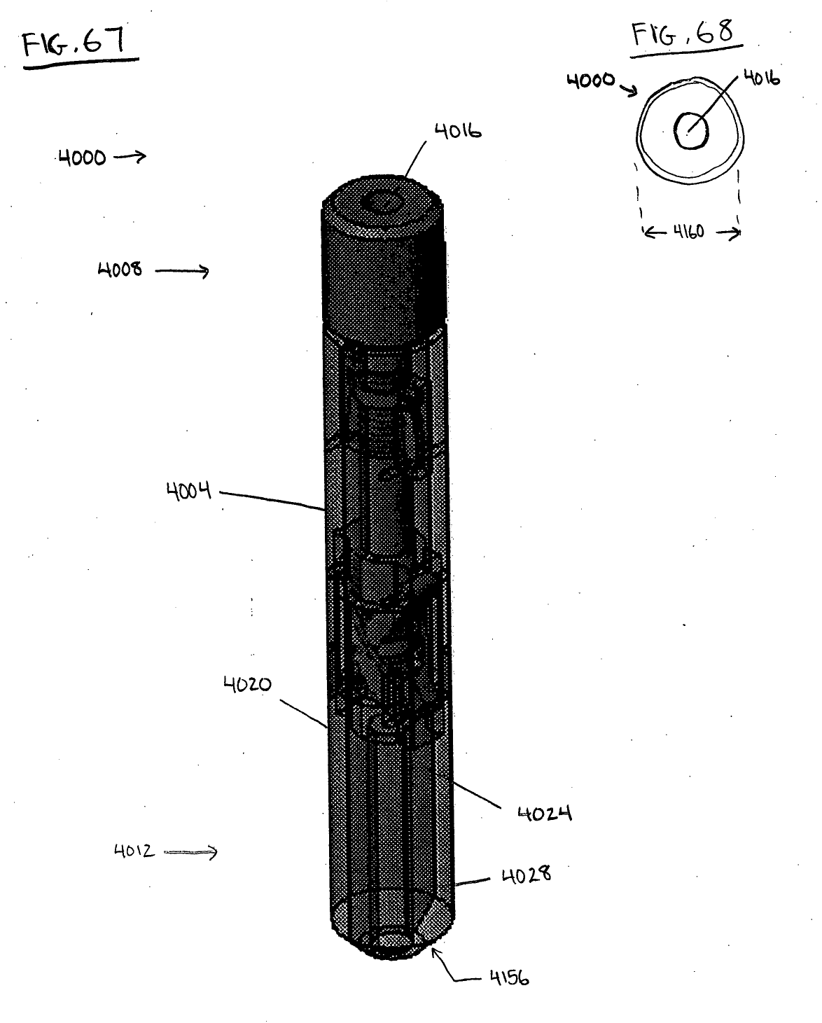

As shown in FIG. 68, in one embodiment having a generally circular configuration in a low profile or un-expanded configuration, a diameter 4160 of the elongate body 4004 in the low profile configuration preferably is between about 8 mm and about 20 mm. In other embodiments, the diameter of the elongate body 4004 can be less than about 8 mm or greater than about 20 mm.

As shown in FIG. 73, in one embodiment having a generally oblong, e.g., oval or elliptical, shaped expanded configuration, a first dimension 4164 of the elongate body 4004 in an expanded configuration preferably is between about 8 mm and about 20 mm. In other embodiments, the first dimension can be less than about 8 mm or greater than about 20 mm. A second dimension 4168 of the elongate body, substantially perpendicular to the first distal dimension 4164, in the expanded configuration preferably is between about 16 mm and about 30 mm. In other embodiments, the second dimension 4168 can be less than about 16 mm or greater than about 30 mm. The terms “substantially perpendicular” as used herein, can mean within about 5 degrees of perpendicular, within about 10 degrees of perpendicular, or within about 15 degrees of perpendicular as viewed from the plan view for some embodiments.

FIG. 77 is a schematic view of another embodiment of a dilator 4200 provided for dilating tissue at a surgical location within a patient. The dilator 4200 is similar to the dilator 4000 except as set forth below. The dilator 4200 preferably can be actuated from a low-profile or unexpanded configuration to an expandable configuration. The dilator 4200 is shown in an expanded configuration in FIG. 77.

The dilator 4200 comprises an elongate body 4204 having a proximal portion 4208, a distal portion 4212, a first side portion 4216, and a second side portion 4220. The first side portion 4216 has a first longitudinal edge 4224. The second side portion 4220 has a second longitudinal edge 4228. The first side portion 4216 and the second side portion 4220 are movable relative to each other such that the first longitudinal edge 4224 and the second longitudinal edge 4228 can be positioned in close proximity to each other (e.g., in the un-expanded configuration) or spaced apart by a selected distance (e.g., in an expanded configuration).

The elongate body 4204 has an outer surface 4232 that can engage and dilate tissue. In some embodiments, the elongate body 4204 has an inner surface 4236, which can at least partially define a passage 4240. As discussed above, the elongate body 4204 is capable of having a low profile configuration and an expanded configuration when positioned within the patient. The cross-sectional area of the expanded tissue opening defined by the outer surface 4232 in the expanded configuration is greater than the cross-sectional area of the tissue opening defined by the outer surface 4232 in the low-profile configuration. Preferably a maximum distance between the first side portion 4216 and the second side portion 4220 in the expanded configuration is greater than a maximum distance between the first side portion 4216 and the second side portion 4220 in the low-profile configuration.

An actuation system 4244 having an actuating device and at least one linkage can be used to expand the dilator 4200. In one embodiment, the dilator 4200 actuation system 4244 comprises a first linkage 4248 and a second linkage 4252 configured to expand or contract the elongate member 4208 in response actuating an actuation device, such as by turning a knob 4256. The linkages can take any suitable arrangement, including any combination of those described herein. In one embodiment, at least one of the two linkages 4248, 4252 is similar to the second linkage 4076. For example, in one arrangement, both of the linkages 4248, 4252 have two crossing members on either side of a coupling member that is configured to translate along an internal structure, such as a rotatable shaft.

FIG. 78 is a schematic view of another embodiment of a dilator 4400 that is similar to the expandable dilators described above, except as differently described below. The dilator 4400 is configured for at least two stages of expansion. As with the dilators described above, the dilator 4400 is configured to be moved from an un-expanded or low profile configuration to an expanded configuration in which tissue is dilated primarily laterally from the device. This operation can be described as a first stage of expansion. The dilator 4400 has a second stage of expansion that preferably further dilates tissue. For example, as shown schematically and discussed further below, the dilator 4400 can be actuated such that elongate portions thereof, which can be blades, tilt relative to a longitudinal axis of the un-expanded device. Such tilting creates increases the dilated space toward the distal end of the dilator 4400 and toward the distal end of a passage that extends between the skin and a spinal location.

More particularly, the dilator 4400 has an un-expanded configuration that is not illustrated but that is analogous to the configuration of the dilator 4000 shown in FIG. 67. The dilator 4400 is provided for dilating tissue at a surgical location within a patient for minimally invasive access to a region of the spine. The dilator 4400 comprises an elongate body 4404 having a proximal portion 4408, a distal portion 4412, a first elongate member 4416, and a second elongate member 4420. The first elongate member 4416 and the second elongate member 4420 are configured to retract tissues to enlarge a passage extending at least partially between the patient's skin and a vertebral location. Such dilation can be used to expose at least a portion of at least one vertebra in the spine of the patient. The dilator 4400 also has a first expanded configuration and a second expanded configuration. The elongate body 4404 has an expanded configuration wherein a recess 4424 is defined at least in part by the first elongate member and the second elongate member 4416, 4420 when the dilator 4400 is in the first expanded configuration. In one embodiment, the first expanded configuration includes a substantially constant dimension recess 4424 along the distal portion 4412. An actuation system 4428 having a shaft 4432 that is threadably engaged with at least one coupling member 4436 can be used to expand the dilator 4400 laterally.

A second expanded configuration is illustrated by the dashed-line representation of the first and second elongate member 4416, 4420. In the second expanded configuration, the size of the recess 4424 varies along the distal portion 4412, e.g., is greater near the distal end of the elongate body 4404. The actuation system 4428 can be configured to actuate the dilator 4400 to the second expanded configuration. Some embodiments of the actuation system 4428 comprise a suitable linkage, such as any of those described herein. Some embodiments comprise at least one coupling member 4436 that acts as a wedge in contact with at least one of the first and second elongate members 4416, 4420. In particular, the coupling member 4436 can be a wedge-shaped member 4438 threadably engaged with the shaft 4432. Rotation of the shaft 4432 causes the wedge-shaped member 4438 to translate along the shaft 4432 into engagement with a ramped surface 4439 on at least one of the first and second elongate members 4416, 4420. When the shaft 4432 is rotated the coupling member 4436 moves up or down the shaft, permitting the elongate body 4404 to expand and contract.

FIGS. 79-84 illustrate another embodiment of a dilator 4500 that is configured to enable expanding the size of the dilator from a low-profile configuration to an expanded configuration.

The dilator 4500 comprises an elongate body 4504 that has a proximal portion 4508 and a distal portion 4512. In some embodiments, the dilator 4500 includes a passage 4516 that extends through the elongate body 4504. The passage 4516 can be formed at or centered on a central longitudinal axis that extends through the elongate body 4504. The dilator 4500 can be configured such that when in an un-expanded position there is no passage 4516 through the elongate body 4504.

When included, the passage 4516 can be formed between a plurality of elongate members that extend along a substantial length of the elongate body 4504. In one embodiment, the dilator 4500 includes a first pair of elongate members 4524A and a second pair of elongate members 4524B. In one arrangement, the elongate members of the first pair 4524A are larger than the elongate members of the second pair 4524B. The elongate body 4504 of the dilator 4500 defines an outer surface 4530 that is configured to engage tissue and to retract tissue to increase the size of a passage through the tissue, whereby an access device can be inserted into the patient to further increase access to a surgical location.

The dilator 4500 includes an actuation system 4532 in one embodiment that moves the first and second pairs of elongate members 4524A, 4524B between an un-expanded configuration and an expanded configuration. In one embodiment, an un-expanded configuration provides a substantially enclosed, circular perimeter along at least a portion of the length of the elongate body 4504. When the dilator 4500 is in this un-expanded configuration, the outer surface comprises a substantially continuous outer surface 4530.

The actuation system 4532 can take any suitable form and in one embodiment includes a cam-actuated expansion arrangement. In one embodiment the actuation system 4532 includes a first pair of cam lobes 4536 configured to engage a corresponding pair of cam surface 4540 located on internal surfaces of the first pair of elongate members 4524A. Engagement of the cam lobes 4536 with the cam surfaces 4540 cause the elongate members 4524A to be moved outwardly such that tissue may be dilated, as discussed further below.

In one embodiment, the cam lobes 4536 are located on a cam shaft 4544 that extends distally from the proximal portion 4508 of the dilator 4500. To facilitate expansion of the dilator 4500, in some embodiments a second set of cam lobes 4536 is provided such that cam lobes for actuating the first pair of elongate member 4524A are located in the proximal and distal portions 4508, 4512 of the dilator 4500. The cam shaft 4544 also can include threaded portions 4548 located at proximal and distal ends thereof that facilitate assembly of the dilator 4500. For example a distal threaded portion 4548 can be engaged with a distal cap member 4552. In one arrangement, the distal cap member 4552 can be received in a cylindrical recess 4556 formed in elongate members 4524A, 4524B. In one embodiment, a proximal threaded portion 4548 can be coupled with an actuation member 4560, which is configured to rotate the cam shaft 4544 to expand the first pair of elongate members 4524A.

In one embodiment, the actuation member 4560 comprises the proximal threaded portion 4548 of the shaft 4544 and an internally threaded structure, e.g., a local nut 4564, to engage the threaded portion 4548. In one embodiment, the lock nut 4564 is received in and engages an internal recess formed in an actuation member bracket 4568. In one embodiment, an actuation member or handle 4572 also is coupled with the actuation member bracket 4568. Preferably the actuation member 4572 is pivotably coupled with the bracket 4568 such that the actuation member can be moved between an actuation position and a low-profile position, as discussed below.

Expansion of the first pair of elongate members 4524A is achieved as follows in the foregoing described embodiment. The actuation member 4572 is moved to the position shown in FIGS. 79-81 by applying a force to the actuation member 4572. The actuation member 4572 preferably is elongate to provide mechanical advantage to increase a force applied to the shaft 4544. The force applied to the shaft 4544 causes the shaft 4544 to rotate, whereby contact between the cam lobes 4536 and the cam surfaces 4540 is provided. The cam lobes 4536 have a transverse extent that is greater than the transverse spacing of the surfaces 4540 on opposite elongate members 4524A. Thus, the contact between the lobes 4536 and the surfaces 4540 pushes the elongate members 4524A outwardly from the shaft 4544.

In some embodiments, the second pair of elongate members 4524B can be separately actuated from the first pair of elongate members 4524A. In one arrangement, the actuation system 4532 includes an actuation member or handle 4576 that is coupled with the second pair of elongate members 4524B. For example, a bracket 4580 can be provided between the actuation member 4576 and the elongate members 4524B. The bracket 4580 is configured such that it can be rotated relative to the shaft 4544. Rotation of the actuation member 4576 and the bracket 4580 causes the second pair of elongate members 4524B to move, e.g., rotate, relative to the shaft 4544. In one arrangement, the shaft 4544 includes cam lobes 4584 configured to engage the elongate members 4524B during such movement, whereby the members move outwardly relative to the shaft 4544. Such outward movement of the elongate members 4524B causes the members to engage tissue to increase the size of a passage extending from an incision to a surgical location. In one arrangement, the range of motion of the actuation member 4576 and the bracket 4580 are limited by providing a channel 4588 in an upper surface of the bracket 4580 which can be engaged by a pin member 4592 that extends between the channel 4588 and the actuation member bracket 4568.

In some embodiments, the dilator 4500 is configured such that the actuation handles 4572, 4576 can be positioned in a low-profile configuration. As discussed above, it may be desirable to apply an access device over the dilator 4500 after the dilator has been expanded. To facilitate this application, the actuation handles 4572, 4576 preferably are configured to be rotated from the positions shown in FIGS. 79-81 to a position in which their longitudinal axes are substantially parallel to a longitudinal axis of the passage 4516. This can be accomplished by providing pin connections between the actuation handle 4572 and the actuation member bracket 4568 and between the actuation handle 4576 and the bracket 4580. In another embodiment, the dilator 4500 can be configured to be low profile after expansion by making the actuation handles 4572, 4576 removable from the brackets 4568, 4580.

FIGS. 85-89 illustrate a dilator 4600 that is similar to the dilator 4000 except as set forth below. The dilator 4600 is configured to be moved between an un-expanded or low-profile configuration and an expanded configuration, as described further below. In this embodiment, the movement between the un-expanded and expanded configurations is provided by a rotation of a lever about an axis that is generally transverse to the longitudinal axis of the dilator 4600. This arrangement is advantageous in that the range of motion of a distal portion of the dilator 4600 is achieved with a relatively small range of motion of the lever, and thus this motion can be achieved quickly to shorten the duration of the dilation phase of a procedure. While generally more compact, embodiments that include rotatable knobs sometimes require more rotation to achieve the same amount of expansion.

In one embodiment, the dilator 4600 includes an elongate body 4604 that has a proximal portion 4608 and a distal portion 4612. As discussed further below, the distal portion 4612 is expanded by an actuation system 4614 that extends between the proximal and distal portions 4608, 4612.

The dilator 4600 includes a passage 4616 that is configured to receive a guidewire or guide pin. The passage 4616 can be defined within a small tube 4618 that is located adjacent to a central longitudinal axis of the dilator 4600. In one embodiment, the tube 4618 is coupled with a stationary handle 4622 of the dilator 4600. The stationary handle 4622 forms a portion of the actuation system 4614, discussed in greater detail below.

The distal portion 4612 of the dilator 4600 includes a plurality of elongate members 4624 that can be moved between un-expanded and expanded positions. In the embodiment shown in FIGS. 85-88, four elongate members 4624 are provided that move diagonally away from each other and away from the tube 4618.

The actuation system 4614 is configured to move the elongate member 4624 away form each other to dilate tissue prior to placement of an access device. In one embodiment, the actuation system 4614 further comprises a movable actuation lever 4628 that can be moved between a position corresponding to a low profile or un-expanded configuration of the dilator 4600, as shown in FIG. 86, and a position corresponding to an expanded configuration of the dilator, as shown in FIGS. 86A and 87. The movable actuation lever 4628 is pivotably coupled with the stationary handle 4622 by a pin-type connection 4630 in one embodiment. The movable actuation lever 4628 is pivotably coupled with an actuating tube 4632 that extends distally from the proximal portion 4608 of the dilator 4600. In one embodiment, a link member 4631 extends between the movable actuation lever 4628 and a coupling 4633 to which the actuating tube 4632 is coupled by a pivot joint 4635. FIGS. 86 and 86A illustrate that rotation of the movable actuation lever 4628 causes upward movement of the link member 4631, which in turn causes upward movement of the coupling 4633 and the actuating tube 4632.

The actuating tube 4632 is disposed concentrically about the small tube 4618 in one embodiment. A link member 4636 extends between a distal portion of the actuating tube 4632 and each of the elongate members 4624. In one embodiment, a linkage is formed by each of the elongate members 4624, the link member 4636 and a hub 4640 mounted to the distal end of the actuating tube 4632. Accordingly, as the actuating tube 4632 is raised, the hub 4640 is raised and the link members 4636 push the elongate members 4624 away from the actuating tube 4632. As the elongate members 4624 away from the actuating tube 4632, tissue is dilated, as discussed above.

In one embodiment, a second set of link members 4636A and a hub 4640A are positioned near the distal end of the tube small tube 4618. Preferably a shoulder 4644 is formed on the tube 4618 and the hub 4640A is mounted distal of the shoulder 4644. Because the tube 4618 is stationary, the hub 4644 also is stationary. Thus, the link members 4636A, the hub 4640A, and each of the elongate members 4624 form a linkage that moves in a similar fashion to the linkage formed by the link members 4636, the hub 4640, the elongate members 4624.

Although the dilator 4600 has been configured to expand by raising the actuating tube 4632, the dilator could be configured to be expanded by lowering the actuating tube 4632. Also, the dilator could be configured to expand by moving the actuation lever 4628 away form the stationary handle 4622 rather than toward it as described above. Where movement away from the stationary handle 4622 is desired, a mechanism can be provided to enable the actuation lever 4628 to be repositioned near the stationary handle 4622 to keep the profile of the proximal end 4608 low so that an access device can more easily be advanced over the dilator.

In another embodiment, a system provides access to a surgical location adjacent the spine. The system comprises any of the embodiments of the dilator disclosed herein for dilating tissue at the surgical location. The dilator comprises an elongate body having a proximal portion, a distal portion, an outer surface, an inner surface, a first elongate member, and a second elongate member. The first and the second elongate members are configured to retract tissues to expose at least a portion of at least one vertebra or otherwise increase or facilitate access to a spinal location. The outer surface is defined at least in part by the first elongate member and the second elongate member. The elongate body has a low profile configuration and an expanded configuration. The cross-sectional area defined by the outer surface in the expanded configuration is greater than the cross-sectional area defined by the outer surface in the low-profile configuration. The system comprises a retractor for providing minimally invasive access to the spine. The retractor can be any of the retractors disclosed herein or in any of the incorporated references. One embodiment of the retractor comprises an elongate body having an outer surface and an inner surface. The inner surface defines a passage extending through the elongate body. The retractor is capable of having a configuration wherein the inner surface of the retractor is positionable over the outer surface of the dilator when the dilator is in the expanded configuration within the patient. The elongate body of the retractor is capable of having a configuration when positioned within the patient wherein the cross-sectional area of the passage at a first location is greater than the cross-sectional area of the passage at a second location, wherein the first location is distal to the second location. The passage is capable of having a configuration through which multiple surgical instruments can be inserted simultaneously to the surgical location when the dilator is withdrawn from the passage.

According to one technique for minimally invasive surgery, any of the dilators described herein is applied through an incision in the skin of a patient in a region of the spine of the patient. In one embodiment, the dilator is inserted over a guidewire or guide pin. After the incision is made, the dilator can be inserted into the incision to prepare the incision to receive a larger access device or retractor, such as those described herein and in the incorporated references. For example, the incision can be dilated using an expandable dilator and the tissue can be cut or stripped away if desired. Thereafter, a larger access device or retractor may be advanced through the incision over the expanded dilator. The dilating retractor can be contracted and withdrawn and the larger access device can remain in patient.

In another application, a method for accessing a surgical location within a patient comprises providing a dilator for insertion into the patient. The dilator has a first elongate member and a second elongate member. The dilator is positioned in a low-profile configuration for insertion into the patient. In the low-profile configuration, the first elongate member is adjacent the second elongate member. The dilator is positioned in an enlarged profile configuration. In the enlarged profile configuration, the first elongate member is spaced from the second elongate member. A retractor is provided for insertion into the patient. The retractor has a proximal portion and a distal portion. The distal portion is coupled with the proximal portion and has an outer surface and an inner surface partially defining a passage. The retractor is positioned in a low-profile configuration for insertion into the patient. The retractor is inserted into the patient to the surgical location over the dilator. The retractor is positioned in an enlarged profile configuration. In the enlarged profile configuration, the retractor is configured such that the cross-sectional area of the passage at a first location is greater than the cross-sectional area of said passage at a second location, wherein the first location is distal to the second location.

In another embodiment, a system provides access to a surgical location adjacent the spine. The system comprises a first dilator for retracting tissue at the surgical location. The first dilator comprises an elongate body having a proximal portion, a distal portion, and an outer surface. In some embodiments, the first dilator has an inner surface defining a bore. The elongate body is configured to retract tissues when inserted within a patient. In some embodiments the first dilator is expandable. In other embodiments, the first dilator is not expandable. The system can comprise a second dilator for retracting tissue at the surgical location. The second dilator comprises an elongate body having a proximal portion, a distal portion, and an outer surface. In one embodiment, the outer surface of the second dilator defines a perimeter larger than the outer surface of the first dilator. In some embodiments, the second dilator has an inner surface defining a bore. The elongate body is configured to retract tissues when inserted within a patient. In some embodiments the second dilator is expandable. In other embodiments, the second dilator is not expandable. In some embodiments the first and/or second dilators can have an oblong or round cross-sectional shape. This may be useful to accommodate a particular shape of an access device.

In one embodiment the first and the second dilators are inserted sequentially, to prepare an incision to receive an access device. In one embodiment of the system, the first and the second dilators are configured to be positioned within the patient at the same time. In one embodiment, the first and the second dilators are configured to be positioned side by side within a patient. In one embodiment, the outer surfaces of the first and the second dilators define perimeters that are generally the same size and/or the same shape. In another embodiment, the first and the second dilator can have different shapes. For example, the first dilator can have a generally round cross-sectional shape, and the second dilator can have a generally crescent cross-sectional shape. The first and the second dilators being configured such that when the second dilator is positioned near the first dilator, the first and the second dilators together have a generally oblong, e.g., oval or elliptical, cross-sectional shape.

FIGS. 79 and 80 illustrate another embodiment of a dilator 4700 that includes first and second dilators that can be positioned side-by-side. The dilator 4700 can include some of the features of the dilators described above. The dilator 4700 is adapted to retract or dilate tissue to prepare tissue to receive a larger access device or retractor. The dilator 4700 includes a first elongate member 4704 that has a generally round configuration and a second elongate member 4708 that has a generally crescent-shaped configuration. Some embodiments have a channel 4712 for placement over a guidewire. When the first elongate member 4704 is inserted, a generally circular shaped passage is formed by dilation of tissue. When the second elongate member 4508 is inserted, the passage expands to a generally oblong shape.

The first and the second elongate members 4704, 4708 can be inserted and removed sequentially, to prepare an incision to receive an access device. In one embodiment, the first member 4704 is configured to be positioned within the patient and then be removed before insertion of the second member 4708. At least one of the first and second member 4504, 4508 can be manipulated from the proximal end to sweep the location to enlarge the passage near the distal end thereof prior to insertion of a retractor over the dilator 4700. In one technique, the second member 4708 is inserted into the patient following the insertion of the first member 4704.

In another embodiment, one or both of the first and the second dilators do not have inner surfaces that define bores. Accordingly, the first dilator is adapted to be inserted into the patient without using a guide pin or guide wire and then removed from the patient. The second dilator is configured to be positioned within the patient following the removal of the first dilator. It will be appreciated that in the above embodiments, although just one or two dilators may be used, third, fourth, or even more dilators with similar features may be used. In some embodiments, dilators can have progressively larger outer diameters.

FIGS. 91-93 illustrate another embodiment of a dilator assembly 4800 that is similar to the dilator 4700 except as set forth below. Also, many of the structures of the dilator assembly 4800 discussed below can be applied to the dilator 4700.

The dilator assembly 4800 includes a first dilator 4804 that is relatively small and that can be inserted first in one technique, as discussed further below. In the illustrated embodiment, the first dilator 4804 is not cannulated (e.g., having a passage formed therethrough configured to receive a guide pin or guide wire). In other embodiments, the first dilator 4804 can be cannulated. The first dilator 4804 can take any suitable configuration. For example, the dilator 4804 can be generally round and can have a suitable size for insertion into an undilated incision. For example, the dilator 4804 can have an outer diameter of about six (6) millimeters.

Preferably the dilator assembly 4800 also includes a second dilator 4808 that is configured to be inserted over the first dilator 4804. The second dilator 4808 preferably has an inner passage that is sized to receive the first dilator 4804. For example, the second dilator 4808 could have an inner diameter slightly larger than six (6) millimeters. In one arrangement, the second dilator 4808 is configured to be inserted in stages. For example, the second dilator 4808 can comprise a first side portion 4812 and a second side portion 4816. The internal perimeter of at least one of the first and second side portions 4812, 4816 preferably has an irregular shape and the outer perimeter of the first dilator 4804 preferably has a matching irregular profile. These matching profiles are referred to below as “puzzle features” because they are configured to fit together like puzzle pieces such that when assembled, the first dilator 4804 and the first and second side portions 4812, 4816 form an integrated unit. In some cases, the puzzle features limit the numbers of ways these structures can be assembled, thus assuring proper assembly. In other embodiments, the puzzle features are identical and thus these structures are largely interchangeable.

The second dilator 4808 could be formed with a single, unitary construction rather than as a plurality of side portion. Also, the second dilator 4808 could be configured with more than two side portions in some embodiments. Preferably the outer size of the second dilator 4808 is selected to provide dilation of tissue without causing excessive trauma or requiring excessive force for insertion. In one embodiment, the outer profile of the second dilator 4808 is round and the outer size is about twelve (12) millimeters.

In one embodiment, the dilator assembly 4800 also includes a third dilator 4820 that can be inserted over the second dilator 4808. The third dilator 4820 preferably comprises a plurality of side portions. In one embodiment, the third dilator 4820 includes a first side portion 4824 and a second side portion 4828, the first and second side portions being configured to be inserted over the second dilator 4808. The first and second side portions 4824, 4828 preferably have internal profiles that match the external profile of the second dilator 4824. For example, the internal profile of the third dilator 4820 can be round, having a diameter that is slightly larger than about twelve (12) millimeters.

As discussed above, in some cases it is desirable to be able to insert a non-circular, e.g., an oblong or oval, access device into a patient for surgical procedures to be performed over an elongated surgical field. Accordingly, in one embodiment, the third dilator 4820 includes a non-circular outer perimeter. In one embodiment, the outer perimeter of the third dilator 4820 is oblong, e.g., oval. In one embodiment, the first and second side portions 4824, 4828 are generally C-shaped segments that can be separately inserted over the second dilator 4808. When assembled, the C-shaped segments form an oval passage having a major dimension of about twenty-nine (29) millimeters and a minor dimension of about twenty-two (22) millimeters. Although described here as the “third dilator”, in some applications one of the internal dilators is eliminated and the second dilator may be one with an oval or oblong outer perimeter.

FIG. 93 illustrates that in some embodiments, puzzle features 4832 can be provided to couple an internal surface of at least one of the dilators 4808, 4820 with an external surface of at least one of the dilators 4804, 4808. The internal and external surfaces so mated are sometimes referred to herein as “mating surfaces.” In one variation, one or more puzzle feature can be provided between side portions of a split dilator, e.g., on one or more of the dilators 4808, 4820. The puzzle features 4832 can take any suitable form, for example, comprising mating or matched structures on an external surface of a smaller dilator and on an internal surface of a larger dilator configured to be inserted over the smaller dilator. The mating features are a channel in one of the surfaces and a protrusion on the other of the two mating surfaces. In one embodiment, at least one of the puzzle features 4832 extend the entire length of the mating surfaces.

Another feature that can be included to make the dilator assembly 4800 more convenient include distal tapered portions 4836A, 4836B, 4836C on the first, second, and third dilators 4804, 4808, and 4820 respectively. These tapered portions ease insertion and are included on many of the other dilator and dilation structures illustrated and described herein. Also, at least one of the dilators 4804, 4808, and 4820 can be provided with a gripping region 4840, which can be a series of circumferential ridges formed near the proximal end of the dilator assembly 4800.

The dilators 4700 and 4800 are advantageous in that they have a simple construction that does not require complicated mechanisms to increase the size of a passage to the surgical location. Also, by forming at least some of the largest dilators with a split construction, the dilation of the passage can be more gradual than if larger dilators completely surrounded the smaller dilators were used. This more gradual increase in size keeps tissue trauma to a minimum while still achieving the required passage size.

In one application, a method for providing treatment at or near a region of the spine of a patient is provided. A first dilator is inserted into the patient through an incision to retract tissue. The first dilator is advanced until a distal end thereof resides at or near a region of the spine. A second dilator is inserted into the patient through the incision to retract tissue. The second dilator is advanced until a distal end thereof resides at or near a region of the spine. An expandable access device is positioned over at least one of the first and the second dilators. The expandable access device is expanded to retract tissue.

In one variation of the application, the first dilator and the second dilator can have an oblong or round cross-sectional shape, either alone or in combination. In another variation, the first dilator and the second dilator are positioned side by side within the patient. In another variation, the first dilator is positioned in and removed from the patient before the second dilator is positioned in the patient. In another variation, the first dilator and the second dilator have inner surfaces defining bores of approximately the same size. In one variation of the application, one or more of the first dilator and the second dilator do not have a bore.

The various devices, methods and techniques described above provide a number of ways to carry out the invention. Of course, it is to be understood that not necessarily all objectives or advantages described may be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof Accordingly, the invention is not intended to be limited by the specific disclosures of preferred embodiments herein.

Except as further described herein, the embodiments, features, systems. devices, materials, methods and techniques described herein may, in some embodiments, be similar to any one or more of the embodiments, features, systems, devices, materials, methods and techniques described in U.S. patent application Ser. No. 10/845389, filed 13 May 2004, entitled “ACCESS DEVICE FOR MINIMALLY INVASIVE SURGERY,” U.S. patent application Ser. No. 10/678744, filed 2 Oct. 2003, entitled “MINIMALLY INVASIVE ACCESS DEVICE AND METHOD,” U.S. patent application Ser. No. 10/926840, filed 26 Aug. 2004, entitled “ACCESS SYSTEMS AND METHODS FOR MINIMALLY INVASIVE SURGERY,” U.S. patent application Ser. No. 10/927633, filed 26 Aug. 2004, entitled “ACCESS SYSTEMS AND METHODS FOR MINIMALLY INVASIVE SURGERY,” U.S. patent application Ser. No. 10/926579, filed 26 Aug. 2004, entitled “ACCESS SYSTEMS AND METHODS FOR MINIMALLY INVASIVE SURGERY, U.S. patent application Ser. No. 10/972987, filed 25 Oct. 2004, entitled “ADJUSTABLE HEIGHT ACCESS DEVICE FOR TREATING THE SPINE OF A PATIENT,” U.S. patent application Ser. No. 11/094822, filed 30 Mar. 2005, entitled “ACCESS DEVICE HAVING DISCRETE VISUALIZATION LOCATIONS,” and PCT Patent Application 04/33088 filed Oct. 4, 2004, entitled “METHODS, SYSTEMS AND APPARATUSES FOR PERFORMING MINIMALLY INVASIVE SPINAL PROCEDURES,” all of which are hereby incorporated by reference herein in their entirety. In addition, the embodiments, features, systems, devices, materials methods and techniques described herein may, in certain embodiments, be applied to or used in connection with any one or more of the embodiments, features, systems, devices, materials, methods and techniques disclosed in the above-mentioned U.S. patent application Ser. Nos. 10/845389, 10/678744, 10/926840, 10/927633, 10/926579, 10/972987, and 11/094822, all of which are hereby incorporated by reference herein in their entireties.

What is claimed is:

1. A method of treating a spine of a patient, the method comprising:
inserting an elongate body into the patient through an incision, the elongate body comprising a distal portion extending along a longitudinal axis, a proximal portion, and an outer surface;
expanding the elongate body to retract tissue beneath the incision;
inserting an access device over the elongate body, the access device comprising a distal portion, a proximal portion, an outer surface, and an inner surface; and
advancing the access device until the distal end resides at or near a region of the spine.

2. The method of claim 1, wherein the elongate body comprises a first elongate member and a second elongate member and movable member at least partially located between the first and second elongate members, the method further comprising:
rotating the proximal portion of the elongate body to move the movable member, whereby the first and second elongate members are moved laterally to expand the elongate body.

3. The method of claim 2, wherein the proximal portion is coupled with a shaft that extend distally therefrom to a location between the first and second members, and wherein rotation of the proximal portion rotates the shaft and articulates a linkage to apply an outward force on the first and second elongate members.

4. The method of claim 1, wherein expanding the elongate body comprises moving a first elongate member and a second elongate member away from the longitudinal axis.

5. The method of claim 4, wherein the first and second elongate members are moved substantially the same amount adjacent a distal end and adjacent a proximal end thereof.

6. The method of claim 4, wherein the first and second elongate members are moved from a low-profile configuration in which the separation between the first and second elongate members is substantially the same adjacent the proximal and distal ends thereof and an expanded configuration in which the separation between the first and second elongate members is greater adjacent the distal end than adjacent the distal end.

7. The method of claim 1, wherein expanding the elongate body comprises moving first and second elongate members between a low-profile configuration in which the first and second elongate members are adjacent to each other and an expanded configuration in which the separation between the first and second elongate members is substantially the same adjacent the proximal and distal ends thereof.

8. The method of claim 1, wherein expanding the elongate body further comprises rotating a cam lobe into engagement with a cam surface of the elongate body.

9. The method of claim 1, further comprising rotating an actuating member about a longitudinal axis of the elongate body to expand the elongate body.

10. The method of claim 1, further comprising rotating an actuating member from a high profile configuration to a low profile configuration to permit the access device to be inserted over the elongate body.

11. The method of claim 10, wherein rotating the actuating member from the high profile configuration to the low profile configuration causes the elongate body to be expanded.

12. The method of claim 1, wherein inserting the elongate body further comprises inserting a first dilator that is symmetrical about two perpendicular axes through the incision and wherein expanding the elongate body further comprises inserting a second dilator portion configured to mate with an outer surface of the first dilator, wherein the perimeter of the first and second dilators is oblong.

13. The method of claim 12, wherein the second dilator portion is configured to mate with the first dilator by having a curved inner surface that matches a curved outer surface of the first dilator.

14. The method of claim 12, wherein the second dilator portion is configured to mate with the first dilator by having at least one puzzle feature located on an inner surface that is configured to engage a puzzle feature located on an outer surface of the first dilator.

15. The method of claim 12, wherein the second dilator portion comprises a first side portion of a second dilator, the second dilator further comprising a second side portion configured to be inserted opposite the first side portion such that the dilated passage is substantially oval shaped.

16. The method of claim 1, wherein after expanding, the outer perimeter of the elongate body defines at least a portion of a circle.

17. The method of claim 1, wherein after expanding, the outer perimeter of the elongate body defines at least a portion of an oval.

18. The method of claim 1, wherein after expanding, the access device conforms to the elongate body.

19. The method of claim 1, wherein the access device has a substantially constant cross-section from the distal portion to the proximal portion.

20. The method of claim 1, further comprising performing a procedure through the access device.

21. The method of claim 1, further comprising removing the elongate body after the access device has been inserted.

22. A system for providing access to a surgical location adjacent to a spine of a patient, comprising:
a dilator comprising a proximal portion, a first elongate member, a second elongate member, and a movable member at least partially located between the first and second elongate members, the proximal portion coupled with and configured to move the movable member to move the dilator to an expanded configuration; and
an access device having a passage extending therethrough, the passage being configured such that the access device can be advanced over the dilator when the dilator is in the expanded configuration.

23. The system of claim 22, wherein when the dilator is in the expanded configuration the dilator comprises an oblong cross-section, and wherein the access device is capable of being configured such that the passage has an oblong cross-section.

24. The system of claim 23, wherein the dilator comprises a low profile configuration comprising a circular cross-section.

25. The system of claim 22, further comprising a shaft extending distally from and coupled with the proximal portion, the movable member further comprising a wedge-shaped member threadably engaged with the shaft, wherein rotation of the shaft causes the wedge-shaped member to translate along the shaft into engagement with a ramped surface on at least one of the first and second elongate members.

26. The system of claim 22, further comprising a shaft extending distally from and coupled with the proximal portion, the movable member further comprising a link member coupled with at least one of the first and second elongate members and with a coupling threadably engaged with the shaft, wherein rotation of the shaft causes the coupling to translate along the shaft to articulate the link member and to move at least one of the first and second elongate members.

27. The system of claim 26, wherein the link member is pivotably coupled with the coupling, the first elongate member, and the second elongate member.

28. The system of claim 26, wherein the link member comprises a first link member, the coupling is a first coupling, and further comprising a second link member and a second coupling, the second link member being located adjacent the proximal end of the dilator and being pivotably coupled with the second coupling, the first elongate member, and the second elongate member, the second link member being located between the first link member and the proximal end of the dilator.

29. The system of claim 22, further comprising a shaft extending distally from and coupled with the proximal portion, the movable member further comprising a cam lobe coupled with the shaft and configured to engage a cam surface on at least one of the first and second elongate members, the engagement of the cam lobe and cam surface causing the dilator to expand.

30. The system of claim 22, wherein the proximal portion comprises a knob configured to be rotated about a longitudinal axis of the dilator to rotate a shaft coupled with the movable member.

31. The system of claim 22, wherein the dilator further comprises a longitudinal axis and an elongate actuation lever configured to be rotated between a first position for moving the movable member and a second position wherein the actuation lever is substantially parallel to longitudinal axis.

32. The system of claim 22, wherein the dilator further comprises third and fourth elongate members, the first elongate member being located on an opposite side of the dilator from the second elongate member and the third elongate member being located on an opposite side of the dilator from the fourth elongate member, the third and fourth elongate members being separately movable from the first and second elongate members.

33. The system of claim 22, wherein when the access device is in an expanded configuration, a maximum distance between the first elongate member and the second elongate member at a first location is greater than a maximum distance between the first elongate member and the second elongate member in the expanded configuration at a second position, wherein the first location is distal to the second location.

34. A system for providing access to a surgical location adjacent to a spine of a patient, comprising:
a dilator assembly comprising:
a first elongate body comprising a first puzzle feature on an outside surface configured to join the first elongate body to another elongate body;
a second elongate body comprising a first side portion and a second side portion, said first and second side portions configured to be inserted over the first elongate body and to substantially surround the first elongate body, at least one of the first and second side portions comprising a second puzzle feature configured to join the second elongate body to another elongate body; and
an access device having a passage extending therethrough, the access device capable of being configured such that the passage has a shape corresponding to the shape of the second elongate body;
wherein the outer perimeter of the second elongate body is configured such that the access device can be advanced over the dilator assembly in use.

35. The system of claim 34, wherein the second elongate body comprises an oblong outer perimeter and the access device is capable of being configured such that the passage has an oblong inner perimeter.

36. The system of claim 34, further comprising a third elongate body comprising: a first side portion and a second side portion, said first and second side portions configured to be inserted over the first elongate body and to substantially surround the first elongate body, the third elongate body comprising a third puzzle feature and a fourth puzzle feature;
wherein the first puzzle feature is configured to engage the third puzzle feature and the second puzzle feature is configured to engage the fourth puzzle feature to joint the first, second, and third elongate bodies together.
